(19) United States
(12) Patent Application Publication
(10) Pub. No.: US 2024/0092934 A1
(43) Pub. Date: Mar. 21, 2024

(54) ASSESSMENT OF CEACAM1 EXPRESSION ON TUMOR INFILTRATING LYMPHOCYTES

(71) Applicant: FAMEWAVE LTD., REHOVOT (IL)

(72) Inventors: Tehila BEN-MOSHE, Koranit (IL); Yair SAPIR, Manof (IL); Ilana MANDEL, Carmiel (IL); Gal MARKEL, Ramat Gan (IL); Rona ORTENBERG, Yokne'am Illit (IL); Jacob SCHACHTER, Givataim (IL); Francis Joseph CARR, Balmedie (GB); Robert George E. HOLGATE, Royston (GB); Timothy David JONES, Babraham (GB)

(73) Assignee: FAMEWAVE LTD., REHOVOT (IL)

(21) Appl. No.: 18/518,788

(22) Filed: Nov. 24, 2023

Related U.S. Application Data
(63) Continuation-in-part of application No. 16/876,726, filed on May 18, 2020, now Pat. No. 11,866,509.
Application No. 16/876,726 is a continuation of application No. 16/700,490, filed on Dec. 2, 2019, now Pat. No. 11,427,647.
Application No. 16/700,490 is a division of application No. 15/306,664, filed on Oct. 25, 2016, now Pat. No. 10,550,196.
(60) Provisional application No. 61/984,786, filed on Apr. 27, 2014.
(60) Provisional application No. 62/099,155, filed on Jan. 1, 2015.

(51) Int. Cl. C07K 16/30; A61K 39/395; C12N 15/62; G01N 33/574

(52) U.S. Cl. CPC C07K 16/3007; A61K 39/39558; C12N 15/62; G01N 33/57492; A61K 35/17

(57) ABSTRACT

Provided are methods and compositions comprising antibodies capable of specific binding to human CEACAM1 molecules, for treating and diagnosing cancer as well as assessing CEACAM1 expression in tumor infiltrating immune cells (TILs) in a biological sample obtained from a [cancer] subject.

### CROSS-REFERENCE TO RELATED APPLICATIONS

The present application is a Continuation-in-Part application of U.S. patent application Ser. No. 16/876,726 filed on May 18, 2020, which is a Continuation application of U.S. patent application Ser. No. 16/700,490 filed on Dec. 2, 2019 which is now U.S. Pat. No. 11,427,647, which is a Divisional application of U.S. patent application Ser. No. 15/306,664 filed on Oct. 25, 2016 which is now U.S. Pat. No. 10,550,196, which is a national stage filing under 35 U.S.C. § 371 of PCT/IL2015/050433, filed on Apr. 27, 2015, and claims the benefit of priority to U.S. Provisional Application Nos. 61/984,786, filed on Apr. 27, 2014 and 62/099,155, filed on Jan. 1, 2015. Each application is incorporated herein by reference in its entirety.

The instant application contains a Sequence Listing which has been submitted via EFS-Web and is hereby incorporated by reference in its entirety. Said Sequence Listing, created on Nov. 23, 2023, is named 010US CIP Sequence listing.xml, and is 83,171 bytes in size.

### FIELD OF THE INVENTION

The present invention is in the fields of cancer immunotherapy and diagnosis and mainly relates to antibodies capable of specific binding to human CEACAM molecules. More specifically, the present invention relates to said antibodies utilized for determining CEACAM1 expression on tumor-infiltrating immune cells, to optimize cancer patient selection and treatment.

### BACKGROUND OF THE INVENTION

Carcinoembryonic antigen-related cell adhesion molecule 1 (CEACAM1), also known as cluster of differentiation 66a (CD66a), is a member of the carcinoembryonic antigen (CEA) gene family and belongs to the immunoglobulin (Ig) superfamily. CEACAM1 is upregulated in T and NK cells upon activation and its homophilic interactions lead to inhibition of lymphocytes cytotoxic effect. Studies of several human tumor types have suggested that the exploitation of the CEACAM1 pathway may permit immune evasion by tumors. Preclinical animal models of tumors have shown that blockade of CEACAM1 interactions by monoclonal antibodies (mAbs) can enhance the immune response to tumors. An estimated 1,660,290 new cases of cancer and 580,350 cancer-related deaths were seen in the United States in 2013.

Checkpoint Immunotherapy blockade has shown to be an exciting new venue of cancer treatment. Immune checkpoint pathways consist of a range of co-stimulatory and inhibitory molecules which work in concert in order to maintain self-tolerance and protect tissues from damage by the immune system under physiological conditions. Tumors take advantage of certain checkpoint pathways in order to evade the immune system. Therefore, the inhibition of such pathways has emerged as a promising anti-cancer treatment strategy (Pardoll, D. M., 2012, Nat Rev Cancer, 12, 252-264). Anti-tumor immunotherapy via CEACAM1 blockade is not limited in principle to any single tumor type, but may have activity in augmenting therapeutic immune response to a number of histologically distinct tumors.

The anti-cytotoxic T lymphocyte 4 (CTLA-4) antibody ipilimumab (approved in 2011) was the first immunotherapeutic agent that showed a benefit for the treatment of cancer patients (Robert et al., 2011, N. Engl. J. Med., Vol. 364, pages 2517-2526). The antibody interferes with inhibitory signals during antigen presentation to T cells. Anti-programmed cell death 1 (PD-1) antibody pembrolizumab (approved in 2014) blocks negative immune regulatory signaling of the PD-1 receptor expressed by T cells (Hamid, 2013, N. Engl. J. Med., Vol. 2, pages 134-144). Blocking antibodies of the PD-1/PL-L1 axis have shown promising results in several clinical trials in patients with various tumor types (Dolan and Gupta 2014, Cancer

Control, Vol. 21, pages 231-237). An additional anti-PD-1 agent has been filed for regulatory approval in 2014 for the treatment of non-small cell lung cancer (NSCLC). Active research is currently exploring many other immune checkpoints, among them: lymphocyte activation gene 3 (LAG3), CD137, OX40 (also referred to as CD134), and killer cell immunoglobulin-like receptors (KIR) (Gelao et al., 2014, Toxins, Vol. 6, pages 914-933).

Humanized antibodies are antibodies from non-human species (e.g. murine antibodies) whose protein sequences have been modified to increase their similarity to antibody variants produced naturally in humans. The process of “humanization” is usually applied to monoclonal antibodies developed for administration to humans, and performed when the process of developing a specific antibody involves generation in a non-human immune system (such as in mice). The protein sequences of antibodies produced in this way are distinct from antibodies occurring naturally in humans, and are therefore immunogenic when administered to human patients. Humanized antibodies are considered distinct from chimeric antibodies, which have protein sequences similar to human antibodies, but carry large stretches of non-human protein.

It is possible to produce a humanized antibody without creating a chimeric intermediate. Direct creation of a humanized antibody can be accomplished by inserting the appropriate CDR coding segments (responsible for the desired binding properties) into a human antibody scaffold, a process known as “CDR grafting”. In general, after an antibody is developed to have the desired properties in a mouse (or another non-human animal), the DNA coding for that antibody's CDRs can be sequenced. Once the precise sequences of the desired CDRs are known, these sequences are inserted into a construct containing the DNA for a human antibody framework.

WO2010/125571 to the present inventors discloses a murine monoclonal antibody (MRG1) produced by a specific hybridoma cell. The mAb is highly selective to CEACAM1 and does not cross-react with other members of the CEACAM family. WO 2013/054331 to the present inventors discloses a chimeric antibody (CM-10), also highly selective to CEACAM1.

Although there is progress in the field of immunotherapy, there remains a constant need for new treatments that are more effective and longer lasting and which involve novel targets and can work either as signal agents or in combination with known therapies in order to eventually generate long durable responses in cancer patients. There is an unmet need to provide humanized antibodies recognizing specific CEACAM proteins which are safer and more potent and can be used diagnostically and therapeutically in diseases involving CEACAM-proteins expression or activation.

### SUMMARY OF THE INVENTION

The present invention provides humanized antibodies that recognize CEACAM1. Selected humanized antibodies according to the present invention contain numerous specific “back-mutations” in their variable region sequences, namely, mutations from the humanized sequence back to the mouse sequence. These back-mutations are made in residues critical for the maintenance of the original antibody's conformation and binding affinity, while having the lowest incidence of potential T cell epitopes, thus minimizing the risk of adverse immune response towards the antibodies.

The present invention is also based on the remarkable results of a clinical trial testing the humanized anti CEACAM1 monoclonal antibody CM-24 in human cancer patients, indicating that CEACAM1⁺ lymphocytes pre-treatment levels are associated with the survival of these patients, when treated with CM-24 and that treatment with anti CEACAM1 prolonged the survival of patients with high levels of pre-treatment CEACAM1⁺ lymphocytes. It is therefore disclosed for the first time that CEACAM1 level on tumor-infiltrating lymphocytes can be used as a biomarker to optimize cancer patient selection and treatment and to optimize treatment.

In order to produce a humanized mAb which recognizes CEACAM1 having specific CDR sequences in desired orientation and conformation and human framework, the inventors of the present invention identified key residues in the human framework (outside the CDR sequences) that affect CDR presentation and designed an array of mutations is these key residues to restore the correct presentation of the CDRs, while minimizing the immunogenicity of the antibodies. The present invention thus provides, for the first time, a high-affinity, non-immunogenic, highly-specific humanized antibody against CEACAM1.

The present invention provides, according to one aspect, a method of treating a cancer subject in need of such treatment, the method comprising: (i) determining the level of CEACAM1 expression on tumor infiltrating or tumor associating immune cells (TILs) in a biological sample obtained from a subject; (ii) comparing the level of CEACAM1 expression on the TILs from the biological sample to a reference value or to a control sample value; and (iii) administering an anti-CEACAM1 mAb or an active fragment thereof to said subject if the level of CEACAM1 expression on TILs in the biological sample is higher than the reference value or the control sample value.

According to certain embodiments, the cancer comprises a solid tumor. According to other embodiments, the cancer is selected from the group consisting of: pancreatic, breast, prostate, melanoma, colorectal, bladder, lung, non-small cell lung carcinoma (NSCLC), non-small cell lung adenocarcinoma (NSCLA), gastrointestinal, thyroid, stomach, ovarian, myeloma and uterine cancer. According to some embodiments, the cancer is selected from pancreatic cancer, colorectal cancer, lung cancer and melanoma. According to specific embodiments, the cancer is a pancreatic cancer.

In certain embodiments, the tumor infiltrating immune cells are lymphocytes. In other embodiments, the tumor infiltrating immune cells are leucocytes.

According to some embodiments, the level of CEACAM1 expression on the TILs in the biological sample is at least 5% of the overall TILs. According to some embodiments, the level of CEACAM1 expression on the TILs in the biological sample is at least 5% overall lymphocytes. According to some embodiments, the level of CEACAM1 expression on the TILs in the biological sample is at least 6%. According to some embodiments, the level of CEACAM1 expression on the TILs in the biological sample is at least 7%. According to some embodiments, the level of CEACAM1 expression on the TILs in the biological sample is at least 8%.

The levels of overall TILs and CEACAM1-expressing TILs may be determined using any detection method know in the art including but not limited to immunofluorescent and immunohistochemical methods. According to some embodiments, the level of total TILs in the biological sample is determined using anti CD3 antibodies. According to some embodiments, the level of overall lymphocytes in the biological sample is determined using anti CD3 antibodies.

In certain embodiments, the level of CEACAM1 expressed on the TILs is determined using an anti-CEACAM1 comprising: (i) a heavy-chain variable region amino-acid sequence comprising the three CDR sequences set forth in SEQ ID NO:1, SEQ ID NO:2 and SEQ ID NO:3; and (ii) a light-chain variable region amino-acid sequence comprising the three CDR sequences set forth in SEQ ID NO:4, SEQ ID NO:5 and SEQ ID NO:6.

According to some specific embodiments, the anti CEACAM1 mAb used for detection of CEACAM1 expression on TILs is MRG1.

According to other specific embodiments, the anti CEACAM1 mAb used for detection of CEACAM1 expression on TILs is a chimeric mAb.

In certain embodiments, the anti-CEACAM1 mAb or an active fragment thereof administered comprises: (i) a heavy-chain variable region amino-acid sequence comprising the three CDR sequences set forth in SEQ ID NO:1, SEQ ID NO:2 and SEQ ID NO:3; and (ii) a light-chain variable region amino-acid sequence comprising the three CDR sequences set forth in SEQ ID NO:4, SEQ ID NO:5 and SEQ ID NO:6.

According to some embodiments, the anti CEACAM1 mAb or an active fragment thereof administered, comprises: (i) a heavy chain variable region sequence selected from the group consisting of: SEQ ID NO:28, SEQ ID NO:29, SEQ ID NO:30, SEQ ID NO:31 and SEQ ID NO:32, and (ii) a light chain variable region sequence selected from the group consisting of: SEQ ID NO:33, SEQ ID NO:34 or SEQ ID NO:35. In certain embodiments, the anti-CAECAM1 mAb administered is selected from a chimeric antibody, a humanized antibody, and a partially humanized antibody. According to some embodiments, the anti-CEACAM1 mAb administered is CM-24.

In other embodiments, the method of treatment comprises administration of at least one additional anti-cancer therapy selected from the group consisting of chemotherapy, radiation, surgery, and immunotherapy. According to certain embodiments, the immunotherapy comprises an inhibitor of an immune checkpoint inhibitor. According to specific embodiments, the immune checkpoint inhibitor is a PD-1 inhibitor or a PD-L1 inhibitor.

According to some embodiments, the biological sample is a biopsy. According to certain embodiments, high expression levels of CEACAM1 on TILs is predictive of prolonged survival in a subject administered with anti-CEACAM1 mAb.

According to certain embodiments, determining the expression level of CEACAM1 on TILs in a biological sample obtained from a subject is preformed prior to treatment. According to some embodiments, administering an anti-CEACAM1 mAb or an active fragment thereof to a cancer subject is preformed if the expression level of CEACAM1 on TILs in the sample is significantly higher than the reference value or the control sample value.

In another aspect, the present invention discloses a pharmaceutical composition comprising an anti-CEACAM1 mAb or an active fragment thereof for use in optimizing treatment in a cancer subject in need of such treatment, the use comprises: (i) utilizing a diagnostic composition to a biological sample obtained from a subject; (ii) determining the level of CEACAM1 expressed on tumor infiltrating immune cells (TILs) in said biological sample; (iii) comparing the level of CEACAM1 expressed on TILs to a reference value or to a control sample value; and (iv) optimizing treatment to said subject based on the level of CEACAM1 expressed on TILs in said sample.

In another aspect, the present invention discloses a diagnostic composition comprising an anti-CEACAM1 mAb or an active fragment thereof for use in optimizing treatment in a cancer subject in need of such treatment, the use comprises: (i) utilizing a diagnostic composition comprising a mAb specific to human CEACAM1 to a biological sample obtained from a subject; (ii) determining the level of CEACAM1 expressed on tumor infiltrating immune cells (TILs) in said biological sample using anti-CEACAM1 mAb or an active fragment thereof;

(iii) comparing the level of CEACAM1 expressed on TILs to a reference value or to a control sample value; and (iv) optimizing treatment to said subject based on the level of CEACAM1 expressed on TILs in said sample.

In certain embodiments, the present invention provides a kit for predicting the response of a subject to an anti-CEACAM1 mAb treatment, comprising said diagnostic composition; and instruction material directing the correlation between the ratio of the CEACAM1 expression on TILs from the biological sample to the reference or control levels.

According to some embodiments, determining the CEACAM1 expression on TILS using the diagnostic composition is preformed prior to treatment.

In certain embodiments, the level of CEACAM1 expressed on the TILs is determined using an anti-CEACAM1 comprising: (i) a heavy-chain variable region amino-acid sequence comprising the three CDR sequences set forth in SEQ ID NO:1, SEQ ID NO:2 and SEQ ID NO:3; and (ii) a light-chain variable region amino-acid sequence comprising the three CDR sequences set forth in SEQ ID NO:4, SEQ ID NO:5 and SEQ ID NO:6.

According to some specific embodiments, the anti CEACAM1 mAb used for detection of CEACAM1 expression on TILs is MRG1.

According to other specific embodiments, the anti CEACAM1 mAb used for detection of CEACAM1 expression on TILs is a chimeric mAb.

The present invention provides, in a different aspect, a method of predicting responsiveness to or selecting a cancer patient amenable to a treatment with an anti-CEACAM1 mAb or active fragment thereof, comprising the steps of: (i) providing a biological sample from the subject; (ii) determining the level of CEACAM1 expression on tumor infiltrating immune cells (TILs) in the biological sample of step (i), and (iii) comparing the level of CEACAM1-expresing TILs to a reference value or to a control sample value, wherein an increase in the level of CEACAM1-expressing TILs relative to the reference value or to the control sample value indicates that the subject is likely to respond therapeutically to the anti-CEACAM1 mAb or active fragment thereof.

In certain embodiments, the anti-CEACAM1 mAb or an active fragment thereof for treatment comprises: (i) a heavy-chain variable region amino-acid sequence comprising the three CDR sequences set forth in SEQ ID NO:1, SEQ ID NO:2 and SEQ ID NO:3; and (ii) a light-chain variable region amino-acid sequence comprising the three CDR sequences set forth in SEQ ID NO:4, SEQ ID NO:5 and SEQ ID NO:6.

According to some embodiments, the anti CEACAM1 mAb or an active fragment thereof for treatment, comprises: (i) a heavy chain variable region sequence selected from the group consisting of: SEQ ID NO:28, SEQ ID NO:29, SEQ ID NO:30, SEQ ID NO:31 and SEQ ID NO:32, and (ii) a light chain variable region sequence selected from the group consisting of: SEQ ID NO:33, SEQ ID NO:34 or SEQ ID NO:35. In certain embodiments, the anti-CAECAM1 mAb for treatment is selected from a chimeric antibody, a humanized antibody, and a partially humanized antibody.

According to certain embodiments, the anti-CEACAM1 mAb is CM-24. According to other embodiments, the cancer comprises a solid tumor. According to some embodiments, the biological sample from the subject is taken prior to treatment.

The present invention provides, according to one aspect, a humanized monoclonal antibody (mAb) which specifically recognizes human CEACAM1, or a fragment thereof comprising at least the antigenic-binding domain, comprising at least one variable region selected from the group consisting of: (i) a heavy-chain variable region comprising CDR1, CDR2 and CDR3 comprising the amino-acid sequences set forth in SEQ ID NO:1, SEQ ID NO:2 and SEQ ID NO:3, respectively; and (ii) a light-chain variable region comprising CDR1, CDR2 and CDR3 comprising the amino-acid sequences set forth in SEQ ID NO:4, SEQ ID NO:5 and SEQ ID NO:6, respectively; wherein at least one of (i) and (ii) contains 1-25 back-mutations of amino acid residues from a human to a murine sequence.

According to some embodiments, the invention provides a humanized monoclonal antibody (mAb) or a fragment thereof, which specifically recognizes human CEACAM1, comprising at least one variable region selected from the group consisting of (i) a heavy-chain variable region amino-acid sequence comprising the CDR sequences set forth in SEQ ID NO:1, SEQ ID NO:2 and SEQ ID NO:3, wherein the heavy-chain variable region amino-acid sequence differs from SEQ ID NO: 57 in 1-25 amino-acid residues in the framework sequences; and (ii) a light-chain variable region amino-acid sequence comprising the CDR sequences set forth in SEQ ID NO:4, SEQ ID NO:5 and SEQ ID NO:6, wherein the light-chain variable region amino-acid sequence differs from SEQ ID NO: 58 in 1-10 amino-acid residues in the framework sequences.

In certain embodiments, the invention provides a humanized monoclonal antibody (mAb) or a fragment thereof, which specifically recognizes human CEACAM1, comprising: (i) the CDR sequences set forth in SEQ ID NO:1, SEQ ID NO:2, SEQ ID NO:3, SEQ ID NO:4, SEQ ID NO:5 and SEQ ID NO:6; (ii) a heavy-chain variable region amino acid sequence that differs in 3-13 amino-acid framework residues from SEQ ID NO: 57; and (iii) a light-chain variable region amino-acid sequence that differs in 3-5 framework amino-acid residues from SEQ ID NO: 58.

According to certain embodiments, the heavy-chain variable region amino-acid sequence differs from SEQ ID NO: 57 in 3-13 amino-acid residues in the framework sequences. According to other embodiments, the light-chain variable region amino-acid sequence differs from SEQ ID NO: 58 in 3-5 amino-acid residues in the framework sequences.

According to other embodiments, the present invention provides a non-fully-humanized monoclonal antibody, comprising (i) a heavy-chain variable region comprising CDR1, CDR2 and CDR3 comprising the amino-acid sequences set forth in SEQ ID NO: 1, SEQ ID NO:2 and SEQ ID NO:3, respectively, and a framework amino-acid sequence that differs in 2 to 9 amino-acids from the amino-acid sequence set forth in SEQ ID NO:9; and/or (ii) a light-chain variable region comprising CDR1, CDR2 and CDR3 comprising the amino-acid sequences set forth in SEQ ID NO:4, SEQ ID NO:5 and SEQ ID NO:6, respectively, and a framework amino-acid sequence that differs in 2 to 4 amino-acids from the amino-acid sequence set forth in SEQ ID NO:13; and analogs, derivatives and antigen-binding-fragments thereof which specifically recognize human CEACAM1.

According to some embodiments, the mAb sequence comprises 1-50 back-mutations to a murine sequence. According to other embodiments, the mAb sequence comprises 2-30 back-mutations to a murine sequence. According to yet other embodiments, the mAb sequence comprises 3-20 back-mutations to a murine sequence. According to other embodiments, the mAb sequence comprises 4-15 back-mutations to a murine sequence. According to other embodiments, the heavy chain of the mAb sequence comprises 1-15 back-mutations to a murine sequence. According to other embodiments, the light chain of the mAb sequence comprises 1-15 back-mutations to a murine sequence. According to other embodiments, the heavy chain of the mAb sequence comprises 2-9 back-mutations to a murine sequence and the light chain of the mAb sequence comprises 2-4 back-mutations to a murine sequence.

According to some specific embodiments, the humanized mAb comprises 1-25 mutations in the heavy chain sequence set forth in SEQ ID NO: 57 from human to murine sequence. According to some embodiments, the heavy chain of the humanized mAb comprises at least one mutation in a residue selected from the group consisting of: V11, R38, M48, V68, M70, R72, T74, S77, R85, R87, T91, Y95 and T115 of SEQ ID NO: 57. According to some embodiments, at least one back-mutation in SEQ ID NO: 57 is selected from the group consisting of: VIIL, R38K, M48I, V68A, M70L, R72A, T74K, S77N, R85S, R87T, T91S, Y95F and T115S.

According to other some specific embodiments, the humanized mAb comprises 1-25 mutations in the light chain sequence set forth in SEQ ID NO: 58 from human to murine sequence. According to some embodiments, the light chain of the humanized mAb comprises at least one mutation in a residue selected from the group consisting of: P44, F71, F73, P80 and Y87 of SEQ ID NO: 58. According to some embodiments, the at least one back-mutation in SEQ ID NO: 58 is selected from the group consisting of: P44V, F71Y, F73L, P80Q, and Y87F.

According to some embodiments, the heavy chain of the humanized mAb comprises at least one mutation in a residue selected from the group consisting of: V11, R38, M48, V68, M70, R72, T74, S77, R85, R87, T91, Y95 and T115 of SEQ ID NO: 57, and the light chain of the humanized mAb comprises at least one mutation in a residue selected from the group consisting of: P44, F71, F73, P80 and Y87 of SEQ ID NO: 58. Each possibility represents a separate embodiment of the present invention.

According to some embodiments, the heavy chain of the humanized mAb comprises at least one mutation in a residue selected from the group consisting of: V68, M70, R72, T74, S77, R85, R87, T91, and Y95 of SEQ ID NO: 57, and the light chain of the humanized mAb comprises at least one mutation in a residue selected from the group consisting of: F71, F73, P80 and Y87 of SEQ ID NO: 58. Each possibility represents a separate embodiment of the present invention.

According to some embodiments, at least one back-mutation in SEQ ID NO: 57 is selected from the group consisting of: V11L, R38K, M48I, V68A, M70L, R72A, T74K, S77N, R85S, R87T, T91S, Y95F and T115S, and the at least one back-mutation in SEQ ID NO: 58 is selected from the group consisting of: P44V, F71Y, F73L, P80Q, and Y87F. Each possibility represents a separate embodiment of the present invention.

According to some embodiments, at least one back-mutation in SEQ ID NO: 57 is selected from the group consisting of: V68A, M70L, R72A, T74K, S77N, R85S, R87T, T91S, and Y95F, and the at least one back-mutation in SEQ ID NO: 58 is selected from the group consisting of: F71Y, F73L, P80Q, and Y87F. Each possibility represents a separate embodiment of the present invention.

In some embodiments, the monoclonal antibody comprises at least one heavy chain framework sequence set forth in a sequence selected from the group consisting of: SEQ ID NO:15, SEQ ID NO: 16, SEQ ID NO: 17, SEQ ID NO:18, SEQ ID NO: 19, SEQ ID NO:20, SEQ ID NO:21, SEQ ID NO:12, and SEQ ID NO:23. In some embodiments, the monoclonal antibody comprises a heavy chain framework sequence set forth in a sequence selected from the group consisting of: SEQ ID NO:18, SEQ ID NO: 19, SEQ ID NO:20, SEQ ID NO:21 and SEQ ID NO:22. Each possibility represents a separate embodiment of the present invention.

In some embodiments, the monoclonal antibody comprises heavy chain framework sequences set forth in SEQ ID NO:7 or SEQ ID NO:15; SEQ ID NO:16 or SEQ ID NO:17; SEQ ID NO: 18, SEQ ID NO:19, SEQ ID NO:20, SEQ ID NO:21 or SEQ ID NO:22; and SEQ ID NO:10 or SEQ ID NO:23. Each possibility represents a separate embodiment of the present invention.

In some embodiments, the monoclonal antibody comprises a heavy chain variable region sequence set forth in a sequence selected from the group consisting of: SEQ ID NO:28, SEQ ID NO:29, SEQ ID NO:30, SEQ ID NO:31 and SEQ ID NO:32. Each possibility represents a separate embodiment of the present invention. In some specific embodiments, the monoclonal antibody comprises the heavy chain variable region sequence set forth in SEQ ID NO:32.

In some embodiments, the monoclonal antibody comprises a light chain framework sequence set forth in a sequence selected from the group consisting of: SEQ ID NO:24, SEQ ID NO:25, SEQ ID NO:26 and SEQ ID NO:27.

In some embodiments, the monoclonal antibody comprises a light chain framework sequence set forth in a sequence selected from the group consisting of: SEQ ID NO:25, SEQ ID NO:26 and SEQ ID NO:27. Each possibility represents a separate embodiment of the present invention.

In some embodiments, the monoclonal antibody comprises a light chain framework sequences set forth in a sequence selected from the group consisting of: SEQ ID NO:11; SEQ ID NO:24; SEQ ID NO:25, SEQ ID NO:26 and SEQ ID NO:27; and SEQ ID NO:14. Each possibility represents a separate embodiment of the present invention.

In some embodiments, the monoclonal antibody comprises the light chain variable region sequence set forth in a sequence selected from the group consisting of: SEQ ID NO:33,

SEQ ID NO:34 and SEQ ID NO:35. Each possibility represents a separate embodiment of the present invention. In some specific embodiments, the monoclonal antibody comprises the light chain variable region sequence set forth in SEQ ID NO:34.

In certain embodiments, the monoclonal antibody comprises: (i) the heavy chain framework sequences set forth in: SEQ ID NO:7 or SEQ ID NO:15; SEQ ID NO: 16 or SEQ ID NO:17; SEQ ID NO:18, SEQ ID NO:19, SEQ ID NO:20, SEQ ID NO:21 or SEQ ID NO:22; and SEQ ID NO:10 or SEQ ID NO:23, and (ii) the light chain framework sequences set forth in: SEQ ID NO:11; SEQ ID NO:24; SEQ ID NO:25, SEQ ID NO:26 or SEQ ID NO:27; and SEQ ID NO:14. Each possibility represents a separate embodiment of the present invention.

In some embodiments, the monoclonal antibody comprises a heavy chain variable region sequence set forth in SEQ ID NO:28, SEQ ID NO:29, SEQ ID NO:30, SEQ ID NO:31 or SEQ ID NO:32; and a light chain variable region sequence set forth in SEQ ID NO:33, SEQ ID NO:34 or SEQ ID NO:35. Each possibility represents a separate embodiment of the present invention. In some embodiments, the monoclonal antibody comprises the heavy chain variable region sequence set forth in SEQ ID NO:32, and the light chain variable region sequence set forth in SEQ ID NO:34.

In some embodiments, the monoclonal antibody comprises the heavy chain variable region sequence set forth in SEQ ID NO:28, and the light chain variable region sequence set forth in SEQ ID NO:33. In some embodiments, the monoclonal antibody comprises the heavy chain variable region sequence set forth in SEQ ID NO:29, and the light chain variable region sequence set forth in SEQ ID NO:33. In some embodiments, the monoclonal antibody comprises the heavy chain variable region sequence set forth in SEQ ID NO:30, and the light chain variable region sequence set forth in SEQ ID NO:33. In some embodiments, the monoclonal antibody comprises the heavy chain variable region sequence set forth in SEQ ID NO:31, and the light chain variable region sequence set forth in SEQ ID NO:33. In some embodiments, the monoclonal antibody comprises the heavy chain variable region sequence set forth in SEQ ID NO:32, and the light chain variable region sequence set forth in SEQ ID NO:33.

In some embodiments, the monoclonal antibody comprises the heavy chain variable region sequence set forth in SEQ ID NO:28, and the light chain variable region sequence set forth in SEQ ID NO:34. In some embodiments, the monoclonal antibody comprises the heavy chain variable region sequence set forth in SEQ ID NO:29, and the light chain variable region sequence set forth in SEQ ID NO:34. In some embodiments, the monoclonal antibody comprises the heavy chain variable region sequence set forth in SEQ ID NO:30, and the light chain variable region sequence set forth in SEQ ID NO:34. In some embodiments, the monoclonal antibody comprises the heavy chain variable region sequence set forth in SEQ ID NO:31, and the light chain variable region sequence set forth in SEQ ID NO:34.

In some embodiments, the monoclonal antibody comprises the heavy chain variable region sequence set forth in SEQ ID NO:28, and the light chain variable region sequence set forth in SEQ ID NO:35. In some embodiments, the monoclonal antibody comprises the heavy chain variable region sequence set forth in SEQ ID NO:29, and the light chain variable region sequence set forth in SEQ ID NO:35. In some embodiments, the monoclonal antibody comprises the heavy chain variable region sequence set forth in SEQ ID NO:30, and the light chain variable region sequence set forth in SEQ ID NO:35. In some embodiments, the monoclonal antibody comprises the heavy chain variable region sequence set forth in SEQ ID NO:31, and the light chain variable region sequence set forth in SEQ ID NO:35. In some embodiments, the monoclonal antibody comprises the heavy chain variable region sequence set forth in SEQ ID NO:32, and the light chain variable region sequence set forth in SEQ ID NO:35.

According to some embodiments, the humanized mAb heavy chain is selected from IgG4 and IgG1 isotype. According to some embodiments, the humanized mAb heavy chain is IgG4 isotype. According to some embodiments, the humanized mAb light chain is kappa isotype. According to some specific embodiments, the humanized mAb comprises a light chain kappa isotype and a heavy chain IgG4 isotype. According to other specific embodiments, the humanized mAb comprises a light chain kappa isotype and a heavy chain IgG1 isotype.

According to some embodiments, the mAb comprises a light chain set forth in SEQ ID NO:52. According to some embodiments, the mAb comprises a heavy chain set forth in SEQ ID NO:53 or SEQ ID NO:59. According to some embodiments, the mAb comprises a light chain set forth in SEQ ID NO: 52 and a heavy chain set forth in SEQ ID NO: 53. According to some embodiments, the mAb comprises a light chain set forth in SEQ ID NO: 52 and a heavy chain set forth in SEQ ID NO: 59.

According to some embodiments, the mAb comprises a light chain variable region set forth in SEQ ID NO: 58 and a heavy chain set forth in SEQ ID NO: 53. According to some embodiments, the mAb comprises a light chain variable region set forth in SEQ ID NO: 58 and a heavy chain set forth in SEQ ID NO: 59. According to some embodiments, the mAb comprises a light chain set forth in SEQ ID NO: 52 and a heavy chain variable region set forth in SEQ ID NO: 57.

The present invention also provides a mAb comprising a light chain variable region set forth in SEQ ID NO: 58 and a heavy chain variable region set forth in SEQ ID NO: 57.

In some embodiments, the humanized mAb or antigen-binding fragment thereof is capable of binding with an affinity of at least about 10-8M to a human CEACAM1 protein. In some embodiments, the humanized mAb or antigen-binding fragment thereof is capable of binding with an affinity of at least about 5×10⁻⁷M to at least one of a human CEACAM3 and human CEACAM5 protein.

The present invention further provides, in another aspect, analogs and/or derivatives of the monoclonal antibody described above, having at least 90% sequence identity with the antigen-binding fragment of said monoclonal antibody.

Fragments of mAbs which recognize CEACAM1, comprising at least an antigen-binding domain, are also included within the scope of the present invention as long as they comprise the above-defined CDR sequences and at least one back-mutation of a human sequence to a murine sequence.

The present invention further provides, in another aspect, isolated polynucleotides encoding a monoclonal antibody described above or a fragment thereof, which specifically recognizes human CEACAM1.

In some embodiments, the isolated polynucleotide sequence comprises a DNA sequences set forth in any one of SEQ ID NOs:44 to 51 encoding a humanized mAb variable region, or analogs thereof having at least 90% sequence identity with said sequences. In some embodiments, the isolated polynucleotide sequence comprises a DNA sequences set forth in SEQ ID NO:54 or SEQ ID NO:55 encoding a humanized mAb heavy chain, or analogs thereof having at least 90% sequence identity with said sequences. In some embodiments, the isolated polynucleotide sequence comprises a DNA sequence set forth in SEQ ID NO:56 encoding a humanized mAb light chain, or analogs thereof having at least 90% sequence identity with said sequences.

The present invention further provides, in another aspect, a plasmid comprising the isolated polynucleotide described above.

The present invention further provides, in another aspect, a pharmaceutical composition comprising a therapeutically effective amount of the monoclonal antibody described above, and a pharmaceutically acceptable carrier, diluent or excipient.

According to some embodiments, the pharmaceutical composition comprises 1-50 mg/ml of humanized mAb to CEACAM1. According to some embodiments, the pharmaceutical composition comprises a basic amino acid. According to some embodiments, the pharmaceutical composition comprises a sugar. According to some embodiments, the pharmaceutical composition comprises a surfactant. According to some embodiments, the pharmaceutical composition comprises a basic amino acid, a sugar and a surfactant. According to some embodiments, the pharmaceutical composition comprises (i) 1-10 mg/ml of basic amino acid; (ii) 10/100 mg/ml of a sugar; (iii) 0.01-1 mg/ml of a surfactant; (iv) 1-50 mg/ml of humanized mAb to CEACAM1, 4-6 mg/ml of basic amino acid, 70-100 mg/ml of a sugar and a 0.1-1 mg/ml of non-anionic surfactant; or (v) 10 mg/ml of humanized mAb to CEACAM1, 4.65 mg/ml of L-Histidine, 82 mg/ml of sucrose and 0.20 mg/ml of polysorbate 20

According to some embodiments, the basic amino acid is selected from the group consisting of: Histidine, Arginine, Lysine and Ornitine. Each possibility represents a separate embodiment of the present invention. According to some embodiments, the composition comprises 1-10, 2-9, 3-7 or 4-6 mg/ml of basic amino acid. Each possibility represents a separate embodiment of the present invention.

According to some embodiments, the sugar is selected from the group consisting of: sucrose, trehalose, glucose, dextrose and maltose. Each possibility represents a separate embodiment of the present invention. According to some embodiments, the composition comprises 10-200, 10-100, 50-150 or 70-100 mg/ml of sugar. Each possibility represents a separate embodiment of the present invention.

According to yet other embodiments, the composition comprises polyol, including but not limited to mannitol and sorbitol. Each possibility represents a separate embodiment of the present invention.

According to some embodiments, the surfactant is a non-anionic. According to some embodiments, the surfactant selected from the group consisting of: polysorbates, sorbitan esters and poloxamers. Each possibility represents a separate embodiment of the present invention. According to some embodiments, the surfactant selected from the group consisting of: polysorbate 20, polysorbate 80. Each possibility represents a separate embodiment of the present invention. According to some embodiments, the composition comprises 0.01-10, 0.01-1, 0.05-5 or 0.1-1 mg/ml of surfactant. Each possibility represents a separate embodiment of the present invention. According to some embodiments, the pharmaceutical composition comprises 4-6 mg/ml of basic amino acid, 70-100 mg/ml of a sugar and a 0.1-1 mg/ml of surfactant.

According to some embodiments, the pharmaceutical composition is in a liquid form and comprises 1-50 mg/ml of humanized mAb to CEACAM1 comprising at least one back-mutation to a murine sequence. According to other embodiments, the pharmaceutical composition is lyophilized. According to some embodiments, the pharmaceutical composition comprises: 10 mg/ml of humanized mAb to CEACAM1, 4.65 mg/ml of L-Histidine, 82 mg/ml of sucrose and 0.20 mg/ml of polysorbate 20.

According to some embodiments, the pharmaceutical composition comprises at least one humanized mAb or fragment defined above and an additional immuno-modulator or a kinase inhibitor. According to some embodiments, a pharmaceutical composition comprising at least one humanized mAb or fragment defined above, and a pharmaceutical composition comprising an additional immuno-modulator or a kinase inhibitor, are used in treatment of cancer by separate administration.

According to some specific embodiments, the additional immuno-modulator is selected from the group consisting of: an anti-human programmed cell death protein 1 (PD-1), PD-L1 and PD-L2 antibody, an activated cytotoxic lymphocyte cell, a lymphocyte activating agent, and a RAF/MEK pathway inhibitor. Each possibility represents a separate embodiment of the present invention. According to some specific embodiments, the additional immuno-modulator is selected from the group consisting of: mAb to PD-1, mAb to PD-L1, mAb to PD-L2, Interleukin 2 (IL-2), lymphokine-activated killer (LAK) cell.

According to some embodiments, the invention provides a pharmaceutical composition comprising a humanized mAb containing back-mutations, as defined above or an antigen-binding fragment thereof, and a pharmaceutical composition comprising a mAb to at least one of human programmed cell death protein 1 (PD-1), PD-L1 and PD-L2 or an antigen-binding fragment thereof, for use in treatment of cancer by separate administration.

According to other embodiments a pharmaceutical composition is provided comprising a humanized mAb to CEACAM defined above or an antigen-binding fragment thereof, and an activated, cytotoxic lymphocyte cell. In certain embodiments, the activated, cytotoxic lymphocyte cell is selected from the group consisting of a LAK cell, a CIK cell, and any combination thereof. Each possibility represents a separate embodiment of the invention. In certain embodiments, the activated, cytotoxic lymphocyte cell is a lymphokine-activated killer (LAK) cell.

In other embodiments, the pharmaceutical composition comprises a humanized mAb to CEACAM1 or an antigen-binding fragment thereof, and a lymphocyte activating agent or a fragment, analog or fusion protein thereof. In certain embodiments, the lymphocyte activating agent is selected from the group consisting of IL-2, IFNγ, an anti-CD3 antibody and fragments, analogs or fusion proteins thereof. In certain embodiments, the lymphocyte activating agent is IL-2 or a fragment, analog or fusion protein thereof. Each possibility represents a separate embodiment of the invention.

In yet other embodiments, the pharmaceutical composition comprises an inhibitor of a kinase selected from the group consisting of a B-Raf kinase mutant, a MEK1 kinase and a MEK2 kinase, and a humanized mAb to human CEACAM1 defined above or an antigen-binding fragment thereof.

According to some specific embodiments, the B-Raf kinase inhibitor attenuates or prevents the phosphorylation of MEK1 or MEK2 by the B-Raf kinase mutant. In certain embodiments, the B-Raf kinase inhibitor attenuates or prevents the dimerization of the B-Raf kinase mutant. In certain embodiments, the MEK1 kinase inhibitor attenuates or prevents the phosphorylation of MAPK by the MEK1 kinase. In certain embodiments, the MEK2 kinase inhibitor attenuates or prevents the phosphorylation of MAPK by the MEK2 kinase.

The present invention further provides, according to other embodiments, an inhibitor of a kinase selected from the group consisting of a B-Raf kinase mutant, a MEK1 kinase and a MEK2 kinase, and a humanized mAb to human CEACAM1 or an antigen-binding fragment thereof, for use in treating cancer. The two active ingredients may be part of one or separate pharmaceutical compositions which can be administered simultaneously or by separate administrations.

The humanized mAb to CEACAM1 according to the present invention and the additional immuno-modulator may be contained in one pharmaceutical composition or in separate compositions for simultaneous or separate administration.

In some embodiments, the pharmaceutical composition is for treatment of a disease or disorder associated with expression, activation or function of a CEACAM protein family member, including but not limited to CEACAM1. Each possibility represents a separate embodiment of the present invention.

In some embodiments, the pharmaceutical composition is for treatment of a disease or disorder associated with CEACAM1 expression, activation or function. In some embodiments, the disease or disorder is a cell proliferative disease or disorder. Each possibility represents a separate embodiment of the present invention. In some embodiments, the cell proliferative disease or disorder is a cancer.

According to some embodiments the cancer is selected from the group consisting of: melanoma, colorectal, bladder, lung, non-small cell lung carcinoma (NSCLC), non-small cell lung adenocarcinoma (NSCLA), gastrointestinal, pancreatic, breast, prostate, thyroid, stomach, ovarian, myeloma and uterine cancer. Each possibility represents a separate embodiment of the present invention.

The present invention further provides, in another aspect, a diagnostic composition comprising at least one mAb or a fragment thereof, which specifically recognizes human CEACAM1, as described above.

According to some embodiments, the mAb or a fragment thereof, which specifically recognizes human CEACAM1 is used for detection of CEACAM1 expression on TILs from a biological sample of a subject diagnosed with cancer or suspected to have cancer.

According to some embodiments, a diagnostic composition is part of a kit.

According to some embodiments, the diagnostic composition is for use in optimizing treatment in a cancer subject in need of such treatment, for predicting responsiveness to an anti-CEACAM1 mAb of active fragment thereof, or for selecting a cancer patient amenable to a treatment with an anti-CEACAM1 mAb of active fragment thereof, the use comprises: (i) providing a biological sample from the subject; (ii) determining the level of CEACAM1 expression on tumor infiltrating immune cells (TILs) in the biological sample of step (i), and (iii) comparing the level of CEACAM1-expresing TILs to a reference value or to a control sample value, wherein an increase in the level of CEACAM1-expressing TILs relative to the reference value or to the control sample value indicates that the subject is likely to respond therapeutically to the anti-CEACAM1 mAb or active fragment thereof, or wherein the CEACAM1 expression on the TILs form the biological sample is used to optimize treatment of said subject.

According to some embodiments, determining the CEACAM1 expression on TILS using the diagnostic composition is preformed prior to treatment.

According to some embodiments, determining CEACAM1 expression on TILs is performed using an anti-CEACAM1 mAb comprising: (i) a heavy-chain variable region amino-acid sequence comprising the three CDR sequences set forth in SEQ ID NO:1, SEQ ID NO:2 and SEQ ID NO:3; and (ii) a light-chain variable region amino-acid sequence comprising the three CDR sequences set forth in SEQ ID NO:4, SEQ ID NO:5 and SEQ ID NO:6.

According to some specific embodiments, the anti CEACAM1 mAb used for detection of CEACAM1 expression on TILs is MRG1.

According to other specific embodiments, the anti CEACAM1 mAb used for detection of CEACAM1 expression on TILs is selected from a chimeric antibody, a humanized antibody, and a partially humanized antibody.

The present invention further provides, in another aspect, a method of preventing, attenuating or treating a disease or disorder associated with expression, activation or function of a CEACAM1 protein, comprising administering to a subject in need thereof a therapeutically effective amount of the pharmaceutical composition described above.

In some embodiments, the disease or disorder is a cancer. In some embodiments, the disease or disorder is an infection, for example a viral infection.

In some embodiments, the isolated antibody contained in the pharmaceutical composition is attached to a cytotoxic moiety.

In certain embodiments, the method described above comprises administering to the subject at least one dose of a humanized mAb to CEACAM1 ranging from 0.01 mg/kg to 10 mg/kg body weight. In certain embodiments, the method described above comprises administering (i) multiple, identical or different, doses of humanized mAb; (ii) multiple escalating doses; or (iii) the pharmaceutical composition once every week, one every 2 weeks, once every 3 weeks, once every 4 weeks, or once every 5 weeks. In certain embodiments, the method described above comprises 1-10 administration cycles, each cycle comprising 2-5 infusions every 1-4 weeks, with a humanized mAb, followed by a 2-8 weeks between each cycle.

In certain embodiments, the method described above further comprises administering a lymphocyte cell or a plurality of lymphocyte cells. In some embodiments, the method described above further comprises administering to the subject CEACAM1-expressing lymphocytes. In some embodiments, the lymphocytes comprise T cells, NK cells or Tumor Infiltrating Lymphocytes (TILs). Each possibility represents a separate embodiment of the present invention. In some embodiments, the lymphocyte cell expresses CEACAM1, PD-1, or both. Each possibility represents a separate embodiment of the present invention. In certain such embodiments, the lymphocyte cell expresses CEACAM1 and PD-1. In some embodiments, the lymphocyte cell is selected from the group consisting of a tumor-infiltrating-lymphocyte (TIL) cell, a lymphokine-activated killer (LAK) cell, a cytokine induced killer (CIK) cell, a T cell, a B cell, an NK cell, and any combination thereof. Each possibility represents a separate embodiment of the present invention. In certain such embodiments, the lymphocyte cell is selected from the group consisting of a tumor-infiltrating-lymphocyte (TIL) cell and a lymphokine-activated killer (LAK) cell. In certain such embodiments, the lymphocyte cell is a tumor-infiltrating-lymphocyte (TIL) cell or a plurality of TIL cells. In certain such embodiments, the lymphocyte cell is a lymphokine-activated killer (LAK) cell or a plurality of LAK cells. In certain embodiments, the lymphocyte cell is activated. In some embodiments, the lymphocyte cell is cytotoxic to a cancer cell. In some embodiments, the cancer cell expresses CEACAM1, PD-L1, PD-L2, or any combination thereof. Each possibility represents a separate embodiment of the present invention. In some embodiments, the cancer cell expresses CEACAM1, PD-L1, or both. Each possibility represents a separate embodiment of the present invention. In some embodiments, the cancer cell expresses CEACAM1, PD-L2, or both. Each possibility represents a separate embodiment of the present invention. In certain such embodiments, the cancer cell expresses CEACAM1 and PD-L1 and PD-L2.

In certain embodiments, the methods described above further comprise administering to the subject a lymphocyte activating agent. According to some embodiments, the lymphocyte activating agent is selected from the group consisting of IL-2, IFNy, and an anti-CD3 antibody. In certain embodiments, the methods described above further comprise administering to the subject an additional anti-cancer composition.

The present invention further provides, in another aspect, a method of immunomodulation, the method comprising contacting a CEACAM1-expressing lymphocyte with the antibodies or fragments thereof described above.

The present invention further provides, in another aspect, a method of inhibiting migration of a CEACAM1-expressing tumor cell, the method comprising contacting said CEACAM1-expressing tumor cell with the antibodies or fragments thereof described above, thereby inhibiting migration of said CEACAM-expressing tumor cell.

The present invention further provides, in another aspect, a method of inhibiting CEACAM1 homotypic or heterotypic protein-protein interaction, the method comprising contacting a CEACAM1-expressing lymphocyte with the antibodies or fragments thereof described above, thereby inhibiting CEACAM1 homotypic or heterotypic protein-protein interaction.

The present invention further provides, in another aspect, a method for increasing the duration or progression of response or survival of a subject having cancer, comprising administering to the subject effective amounts of a composition comprising a monoclonal antibody as described above, and an anti-neoplastic composition, wherein said anti-neoplastic composition comprises at least one chemotherapeutic agent, whereby the co-administration of the antibody and the anti-neoplastic composition effectively increases the duration or progression of survival.

The method of the present invention may comprise administering a pharmaceutical composition defined above together with additional anti-cancer composition. According to a specific embodiment the anti-cancer composition comprises at least one chemotherapeutic agent. According to other specific embodiments, the anti-cancer composition comprises an immuno-modulatory agent. The anti-cancer agent, which could be administered together with the antibody according to the present invention, or separately, may comprise any such agent known in the art exhibiting anticancer activity.

According to some embodiments, a method of treating cancer is provided comprising administering to a subject in need thereof a pharmaceutical composition comprising a humanized antibody which recognizes CEACAM1 and comprises back-mutations to a murine sequence, and a pharmaceutical composition comprising an additional immuno-modulator or a kinase inhibitor.

According to some embodiments, the immuno-modulator is selected from the group consisting of: mAb to PD-1, mAb to PD-L1, mAb to PD-L2, Interleukin 2 (IL-2), lymphokine-activated killer (LAK) cell and the kinase inhibitor is a B-Raf/MEK inhibitor.

In some embodiments the method comprises administration of two or more pharmaceutical compositions. In some embodiments, the administration of two or more of the pharmaceutical compositions is done simultaneously. In some embodiments of the method, the administration of two or more of the pharmaceutical compositions is done sequentially. In some embodiments of the method, the additional immuno-modulator is administered before the humanized mAb to human CEACAM1 or the antigen-binding fragment thereof. In some embodiments of the method, the immuno-modulator is administered simultaneously with the mAb to human CEACAM1 or the antigen-binding fragment thereof. In some embodiments of the method, the immuno-modulator is administered after the humanized mAb to human CEACAM1 or the antigen-binding fragment thereof.

In some embodiments, the method comprises administering to said patient a pharmaceutical composition comprising a monoclonal antibody to human CEACAM1 or an antigen-binding fragment thereof, and a pharmaceutical composition comprising a monoclonal antibody to human PD-1 or an antigen-binding fragment thereof. Each possibility represents a separate embodiment of the present invention.

According to yet other embodiments, the methods described above of treating a patient having cancer, comprises the step of administering to the patient a pharmaceutical composition comprising an inhibitor of a kinase selected from the group consisting of B-Raf kinase mutant, a MEK1 kinase and a MEK2 kinase, and a pharmaceutical composition comprising a monoclonal antibody to human CEACAM1 or an antigen-binding fragment thereof, wherein the cancer cells express a B-Raf kinase mutant, thereby treating the cancer.

In some embodiments, the methods described above for treating cancer further comprises the step of administering to said patient a pharmaceutical composition comprising a lymphocyte cell. In some embodiments, the administration of the pharmaceutical composition comprising a lymphocyte cell is done simultaneously with at least one of the pharmaceutical compositions comprising antibodies. In some embodiments of the method, the administration of the two or more pharmaceutical compositions is done sequentially.

In some embodiments, the lymphocyte cell is pre-incubated with a humanized mAb to human CEACAM1, with an antigen-binding fragment thereof or with the additional immuno-modulator. Each possibility represents a separate embodiment of the present invention.

A pharmaceutical composition according to the present invention may be administered by any suitable means, such as intranasally, subcutaneously, intramuscularly, intravenously, intra-arterially, intraarticulary, intralesionally orally, or topically. According to some embodiments, the pharmaceutical composition is administered parenterally. According to some specific embodiments, intravenous (i.v.), administration is utilized.

A method according to the present invention, of treating cancer or other CEACAM1-associated disease or disorder, comprises according to some embodiments, administering to a subject in need thereof at least one dose of a humanized mAb to CEACAM1 ranging from 0.01 mg/kg to 10 mg/kg body weight.

According to some embodiments, the at least one dose is selected from the group consisting of: 0.01-0.1 mg/kg; 0.1-1 mg/kg; 1-10 mg/kg; and 10-50 mg/kg.

According to some embodiments, the method comprises administering of multiple doses of humanized mAb, wherein the multiple doses are identical or different. According to some embodiments, the method comprises administering multiple escalating doses. According to some embodiments, the method comprises at least one cycle of administration for at least 12 weeks.

According to other embodiments the treatment duration is 12-50 weeks. According to some specific embodiments the treatment duration is selected from the group consisting of: 12-20 weeks, 20-30 weeks and 30-50 weeks. According to yet other embodiments, the treatment regimen comprises several administration cycles each for at least 12 weeks.

According to some embodiments, the treatment regimen comprises 1-8 cycles, each cycle comprises 4 infusions of the humanized anti CEACAM mAb for a duration of at least 4 weeks. According to some embodiments the treatment regimen comprises 2-6 cycles.

According to some embodiments, administration is once every week, one every 2 weeks, once every 3 weeks, once every 4 weeks, or once every 5 weeks. Each possibility represents a separate embodiment of the present invention.

According to some embodiments, a treatment regimen comprises 1-10 cycles, each cycle comprising 2-5 infusions every 1-4 weeks, with a humanized mAb according to the invention, followed by a 2-8 weeks between each cycle.

According to some embodiments a dose escalation regimen is provided comprising administration starting with 0.01 mg/kg, and continuing to 0.03 mg/kg, 0.1 mg/kg, 0.3 mg/kg, 1 mg/kg, 3 mg/kg, and 10 mg/kg. According to yet other embodiments, the treatment regimen comprises 6 cycles of 4 infusions each administered every 2 weeks.

The present invention further provides, in another aspect, a method for diagnosing a cancer in a subject in need thereof, the method comprising contacting a biological sample derived or obtained from said subject with the diagnostic composition described above, wherein a complex formation beyond a predetermined threshold is indicative of the cancer in said subject.

The present invention further provides, in another aspect, a method for determining the expression of CEACAM1, the method comprising contacting a biological sample with the antibodies or fragments thereof described above, and measuring the level of immune complex formation. According to some embodiments, the method comprises comparing said level of immune complex to a standard curve obtained from known amounts of CEACAM1.

The present invention further provides, in another aspect, a method for diagnosing a disease or disorder associated with a CEACAM protein expression, comprising the steps of incubating a biological sample with a monoclonal antibody as described above; detecting the bound CEACAM protein using a detectable probe; comparing the amount of bound CEACAM protein to a standard curve obtained from reference samples containing known amounts of CEACAM protein; calculating the amount of the CEACAM protein in the biological sample from the standard curve; and comparing the amount of CEACAM protein to a normal CEACAM protein amount.

According to some embodiments, a humanized mAb according to the invention is used as a predictive biomarker associated with anti CEACAM1 treatment, based on levels of expression of CEACAM1 in tumor specimens prior to treatment. The expression of CEACAM1 levels is determined using methods known in the art utilizing a mAb according to the invention.

According to other embodiments, a mAb that recognizes human CEACAM1 is used as a predictive biomarker associated with anti CEACAM1 treatment, based on levels of expression of CEACAM1 in tumor-associated immune cells. The expression of CEACAM1 levels is determined using methods known in the art utilizing mAbs.

According to some embodiments, the immune cells are selected from myeloid and lymphoid immune subsets in the tumor and the tumor microenvironment. In some embodiments, the tumor-associated immune cell is a lymphocyte cell selected from the group consisting of a tumor-infiltrating-lymphocyte (TIL) cell, a lymphokine-activated killer (LAK) cell, a cytokine induced killer (CIK) cell, a T cell, a B cell, an NK cell, and any combination thereof.

The present invention further provides, in an aspect, the use of a monoclonal antibody as described above, for diagnosis, prevention or treatment of a cell proliferative or angiogenesis-related disease or disorder or an infection.

The present invention further provides, in an aspect, the use of a monoclonal antibody as described above, for preparation of a medicament for treatment of a disorder or disease associated with expression or activation of a CEACAM protein.

The present invention further provides, in an aspect, the use of a monoclonal antibody as described above, for preparation of a diagnostic composition for the diagnosis of a cell proliferative or angiogenesis-related disease or disorder or an infection.

The present invention further provides, in an aspect, a partly-humanized monoclonal antibody, comprising a heavy-chain variable region comprising a framework amino-acid sequence set forth in SEQ ID NO:18, SEQ ID NO: 19, SEQ ID NO:20, SEQ ID NO:21 or SEQ ID NO:22, or a light-chain variable region comprising a framework amino-acid sequence set forth in SEQ ID NO:25, SEQ ID NO:26 or SEQ ID NO:27.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 is a structural model of the chimeric anti-CEACAM-1 antibody (CM-10) V regions in top view (A) and in stereo side views (B), produced by the protein structure homology-modelling program Swiss PDB.

FIG. 2 is a Coomassie Blue-stained SDS-PAGE gel of Protein A-purified antibodies. Approximately 2 μg of each sample was loaded on a NuPage 4-12% Bis-Tris gel (Invitrogen cat. no. NP0322BOX) and run at 200 V for 40 min. (A) IgG1 CDR-grafted variants; (B) IgG4 (S241P) CDR-grafted variants. Fermentas Pageruler Plus (SM1811) was used as molecular weight standard (containing reference bands at 10, 25, and 70 kDa). The samples were numbered as follows:

| No. | 1 | 2 | 3 | 4 | 5 | 6 | 7 | 8 |
|---|---|---|---|---|---|---|---|---|
| V_{H}/V_{L} | VH1/VK1 | VH1/VK2 | VH1/VK3 | VH2/VK1 | VH2/VK2 | VH2/VK3 | VH3/VK1 | VH3/VK2 |
| No. | 9 | 10 | 11 | 12 | 13 | 14 | 15 | 16 |
| V_{H}/V_{L} | VH3/VK3 | VH4/VK1 | VH4/VK2 | VH4/VK3 | VH5/VK1 | VH5/VK2 | VH5/VK3 | Chimeric |
| | | | | | | | | IgG |

FIG. 3 is a graphic illustration of the results of recombinant human CEACAM-1 competition binding ELISA assays. Varying concentrations of purified humanized IgG antibody variants were competed against a constant concentration of biotinylated anti-CEACAM-1 IgG (chimeric CM-10 IgG1): (A) IgG1 Variants; and (B) IgG4 (S241P) variants. Bound, biotinylated chimeric CM-10 was detected using streptavidin HRP and TMB substrate.

FIG. 4 represents synergistic effects of anti-CEACAM1 and anti-PD-1 antibodies on the cytotoxicity of human TIL cells against human melanoma cells. TIL cells were incubated with various concentrations of a humanized mAb to human CEACAM1 (dashed black line, sphere marker), a mAb to human PD-1 (solid gray line, rectangular marker) or a combination of both antibodies (solid black line, triangle marker). IFN-γ-treated melanoma cells were added for an overnight incubation. Results represent an average of % cytotoxicity ±SE as determined by classical LDH release assay from triplicate wells per treatment. * P<0.05 paired T-test compared to the monoclonal antibody to human CEACAM1 only.

FIGS. 5A-B Synergistic effects of anti-CEACAM1 and anti-PD-1 antibodies on Granzyme B levels and the cytotoxicity of human TIL cells against human melanoma cells when anti-PD-1 antibodies are added prior to the addition of anti-CEACAM1 antibodies. Human melanoma cells were grown in the presence of IFN-ã to induce PD-L1 expression. Human TIL cells were incubated with medium only (black), non-specific IgG antibody (white), various concentrations of a monoclonal antibody to human CEACAM1 (vertical lines), a monoclonal antibody to human PD-1 (horizontal lines) or a combination of both antibodies (dots). (A) Results represent an average of % cytotoxicity ±SE as determined by classical LDH release assay from triplicate wells per treatment. * P≤0.05 paired T-test compared to a-PD-1 only. (B) Results represent Granzyme B levels ±SE as determined by commercial Granzyme B ELISA kit from triplicate wells per treatment.

FIG. 6. Synergistic effects of anti-CEACAM1 and anti-PD-1 antibodies on the cytotoxicity of human LAK cells against human melanoma cells when anti-PD-1 antibodies are added prior to the addition of anti-CEACAM1 antibodies. Human melanoma cells were grown in the presence of IFN-ã to induce PD-L1 expression. Human LAK cells generated by activation of PBMCs from a healthy human donor with IL-2 were incubated with medium only (white), non-specific IgG antibody (black), various concentrations of a monoclonal antibody to human CEACAM1 (vertical lines), a monoclonal antibody to human PD-L1 (horizontal lines) or a combination of both antibodies (dots). Results represent an average of % cytotoxicity ±SE as determined by classical LDH release assay from triplicate wells per treatment. * P≤0.05 paired T-test compared to a-PD-L1 only. Combination index was calculated as described above.

FIG. 7. Treatment with anti-CEACAM1 antibodies increases PD-L1 expression on target cancer cells. NK cells (NK92MI) were incubated with or without CM-24 (10 μg/ml), followed by the addition of human melanoma cells (SKMEL28). The cells were incubated for 24, 48 and 72 hours and PD-L1 levels were measured at each time point by FACS analysis. Mean ratio of anti-PD-L1 compared to an appropriate isotype control for the indicated treatments at the different time points.

FIG. 8. Synergistic effects of anti-CEACAM1 and anti-PD-1 antibodies on tumor progression in immuno-competent mice. Murine lymphoma cells were implanted subcutaneously in the abdomen of BALB/C mice (Day 1). On days 10, 15 and 20, mice were intravenously administered with either PBS (dashed black line, empty circles), an anti-murine CEACAM1 antibody (solid gray line, gray rectangles), an anti-murine PD-1 antibody (solid gray line, gray triangles) or a combination of both antibodies (solid black line, black spheres).

FIG. 9. Anti-CEACAM1 antibodies increase the cytotoxicity of human LAK cells against human melanoma cells. Human LAK cells were incubated with CM-24 in different concentrations for 30 minutes at 37° C. Human melanoma cancer cells (SKMEL28) were added for an incubation of 24 hours. Results represent an average of % cytotoxicity ±SE as determined by classical LDH release assay from triplicate wells per treatment. * P≤0.05 paired T-test compared to effectors +target cells with medium only.

FIG. 10. Anti-CEACAM1 antibodies increase the cytotoxicity of human LAK cells against a human pancreatic cancer cells T3M4. Human LAK cells were incubated with CM-24 in different concentrations for 30 minutes at 37° C. Human pancreatic cancer cells T3M4 were added for an incubation of 24 hours. Results represent an average of % cytotoxicity ±SE as determined by classical LDH release assay from triplicate wells per treatment. * P<0.05 paired T-test compared to effectors +target cells with medium only.

FIG. 11. Anti-CEACAM1 antibodies enhance IFN-γ secretion of human LAK cells in the presence of human cancer cells. Human LAK cells were incubated with CM-24 in different concentrations for 30 minutes at 37° C. Human lung cancer cells H358 (11A) or H460 (11B) were added for an incubation of 24 hours. IFN-γ secretion was measured by ELISA. Results represent the mean +S.E of Granzyme B release values from 3 repeats per treatment. *P<0.05 paired T-test, compared to effectors +target cells with medium only.

FIGS. 12A and 12B presents the correlation between expression of CEACAM1 types CEACAM1-long (A) and CEACAM1-short (B), and resistance to inhibitors of B-Raf mutants in cancer cells.

FIG. 13A presents pictograms of lungs removed from mice engrafted with melanoma cells and treated according to the treatment groups indicated in the figure; FIG. 13B presents the average tumor weight of each treatment group; and FIG. 13C presents the number of lesions per mouse of each treatment group.

FIG. 14 is a bar histogram presenting the percentage of CEACAM1 receptor occupancy in TIL cells isolated from the lung lesions model.

FIGS. 15A and 15B represent the amino acid sequences of the light and heavy chain of the back-mutated humanized anti CEACAM1 mAb, denoted CM-24, currently in clinical trial. FIG. 15A contains the light chain sequence (SEQ ID NO: 52) wherein amino acid residues 1-107 are the variable region including CDRs and amino acid residues 108-214 (in bold) are kappa light chain constant region. FIG. 15B representing the heavy chain sequence (SEQ ID NO: 53), amino acid residues 1-120 are variable region, amino acid residues 121-447 (in bold) are IgG4 heavy chain constant region, and the predicted N-glycosylation site (Asparagine 297) is underlined. FIG. 15C representing the heavy chain sequence (SEQ ID NO: 59), amino acid residues 1-121 are variable region, amino acid residues 122-450 (in bold) are IgG1 heavy chain constant region, and the predicted N-glycosylation site (Asparagine 300) is underlined.

FIG. 16 is a swimmer plot of overall survival for patients with low (0-5%, gray) and high (>8%, black) CEACAM1⁺ lymphocytes in pre-treatment biopsies of eight evaluable PDAC patients of clinical study FW2020-01 Part A (treated with CM-24 and Nivolumab). CEACAM1 expression was assessed by immunohistochemical analysis.

FIG. 17 is a swimmer plot of overall survival for patients with low (0-2%, gray) and high (10%, black) CEACAM1⁺ lymphocytes in pre-treatment biopsies of six evaluable PDAC patients of clinical study FW2020-01 Part C2 (treated with CM-24, Nivolumab and chemotherapy[/Nal-IRI/5FU/LV). CEACAM1 expression was assessed by immunohistochemical analysis.

FIGS. 18A-C CEACAM1⁺ lymphocytes and lymphocytes content analysis of fourteen patients of clinical study FW2020-01 Parts A and C2. CEACAM1 and overall lymphocytes expression were assessed by immunohistochemical analysis. FIG. 18A is a swimmer plot of overall survival for patients with low (0-5%, gray) and high (≥8%, black) CEACAM1⁺ lymphocytes in pre-treatment biopsies of fourteen patients. FIG. 18B shows Kaplan-Meir curves stratified by the levels of CEACAM1⁺ lymphocytes, low (0-5%, gray) and high (≥8%, black). The survival distributions were compared using the likelihood ratio test. FIG. 18C shows Kaplan-Meir curves stratified by the levels of overall lymphocytes (CD3+). The survival distributions of patients with low (gray curve) vs high (black curve) lymphocyte content in pre-treatment biopsies (threshold ≥2%) were compared using the likelihood ratio test.

FIG. 19A and 19B show overall survival of 92 commercial pancreatic ductal adenocarcinoma (PDAC) tumor microarray (TMA) patients' samples. CEACAM1 and overall lymphocytes expression were assessed by immunohistochemical analysis. FIG. 19A presents a Kaplan-Meier estimate according to low (gray curve) and high (black curve) (threshold ≥8%) CEACAM1⁺ lymphocyte levels. FIG. 19B presents a Kaplan-Meier estimate according to low (gray curve) and high (black curve) overall lymphocytes (CD3+) levels (threshold ≥2%).

### DETAILED DESCRIPTION OF THE INVENTION

The present invention discloses non-fully humanized monoclonal antibodies which recognize CEACAM1. Advantageously, the antibodies of the invention are almost fully humanized, thus avoiding the risk of adverse immune response towards the antibodies and are therefore safe for in-vivo use in humans. The antibodies of the invention are characterized by having unique CDR sequence and novel non-fully humanized framework sequences and design. The unique properties of the monoclonal antibodies of the present invention, broaden their therapeutic utility for treatment and diagnosis of additional types of malignancies and various infections. More specifically, the monoclonal antibodies provided by the present invention have specific combinations of CDRs and non-fully-humanized framework sequences, and possess unique properties and improved safety and potency over known non-human anti-CEACAM1 antibodies.

The unique properties of the antibodies provided by the present invention confer several advantages to the use of these antibodies in human, specifically in applications requiring long-term or repeated administration, when other, non-human antibodies cannot be administered in the fear of eliciting an immunogenic response towards the non-human antibodies themselves. Avoiding such an immune response becomes more crucial when the treated person is a patient inflicted with a disease, where further aggravating the patient's health should be avoided. The present invention yet further provides, in another aspect, a non-fully-humanized monoclonal antibody, comprising (i) a heavy-chain variable region comprising CDR1, CDR2 and CDR3 comprising the amino-acid sequences set forth in SEQ ID NO:1, SEQ ID NO:2 and SEQ ID NO:3, respectively, and a non-CDR (framework) amino-acid sequence that differs in 2 to 9 amino-acids from the amino-acid sequence set forth in SEQ ID NO:9; and/or (ii) a light-chain variable region comprising CDR1, CDR2 and CDR3 comprising the amino-acid sequences set forth in SEQ ID NO:4, SEQ ID NO:5 and SEQ ID NO:6, respectively, and a non-CDR amino-acid sequence that differs in 2 to 4 amino-acids from the amino-acid sequence set forth in SEQ ID NO:13; and analogs, derivatives and antigen-binding-fragments thereof which specifically recognize human CEACAM1.

In the interest of clarity, it should be emphasized that the variable regions of the antibodies provided by the present invention comprise (a) the CDR sequences previously described by the inventors of the present invention, and (b) framework sequences, also denoted herein non-CDR sequences, at least one of which is different in at least one residue from a corresponding fully human framework sequence.

In certain embodiments, the phrase “a sequence which differs from another sequence” as used herein means a sequence which contains a substitution of at least one amino-acid, an insertion of at least one amino-acid, a deletion of at least one amino-acid, or any combination thereof, in comparison to a respective sequence. In certain embodiments, the phrase “a sequence which differs from another sequence” as used herein means a sequence which contains a substitution of at least one amino-acid in comparison to a respective sequence. The term “non-CDR sequence” as used herein refers framework sequence, namely, any amino-acid sequence comprised in a variable region of an antibody, which is not a CDR sequence identified by the present invention. Examples of non-CDR sequence include sequences preceding or adjacent to CDR1, sequences between CDR1 and CDR2, sequences between CDR2 and CDR3, and sequences following or adjacent to CDR3.

Since the variable regions of the antibodies provided by the present invention differ in at least one amino-acid from the variable regions fully human antibodies, they are also labeled “non-fully-humanized” and “non-fully-human” antibodies.

The term “CEACAM1” is used to refer to the protein product of the CEACAM1 gene e.g., NP_001020083.1, NP_001703.2. In humans, 11 different CEACAM1 splice variants have been detected so far. Individual CEACAM1 isoforms differ with respect to the number of extracellular immunoglobulin-like domains (for example, CEACAM1 with four extracellular immunoglobulin-like domains is known as CEACAM1-4), membrane anchorage and/or the length of their cytoplasmic tail (for example, CEACAM1-4 with a long cytoplasmic tail is known as CEACAM1-4L and CEACAM1-4 with a short cytoplasmic tail is known as CEACAM1-4S). The N-terminal domain of CEACAM1 starts immediately after the signal peptide and its structure is regarded as IgV-type. For example, in CEACAM1 annotation P13688, the N-terminal IgV-type domain is comprised of 108 amino acids, from amino acid 35 to 142. This domain was identified as responsible for the homophilic binding activity (Watt et al., 2001, Blood. 98, 1469-79). All variants, including these splice variants are included within the term “CEACAM1”.

The terms “anti-CEACAM1 antibody”, “an antibody which recognizes CEACAM1”, “an antibody against CEACAM1” and “an antibody to CEACAM1” are interchangeable, and used herein to refer to an antibody that binds to the CEACAM1 protein with sufficient affinity and specificity.

The term “antigen” as used herein refers to a molecule or a portion of a molecule capable of eliciting antibody formation and being bound by an antibody. An antigen may have one or more than one epitope. The specific reaction referred to above is meant to indicate that the antigen will react, in a highly selective manner, with its corresponding antibody and not with the multitude of other antibodies which may be evoked by other antigens. An antigen according to the present invention is a CEACAM1 protein or a fragment thereof.

The term “antigenic determinant” or “epitope” as used herein refers to the region of an antigen molecule that specifically reacts with a particular antibody. Peptide sequences derived from an epitope can be used, alone or in conjunction with a carrier moiety, applying methods known in the art, to immunize animals and to produce additional polyclonal or monoclonal antibodies. Isolated peptides derived from an epitope may be used in diagnostic methods to detect antibodies and as therapeutic agents when inhibition of said antibodies is required.

Antibodies, or immunoglobulins, comprise two heavy chains linked together by disulfide bonds and two light chains, each light chain being linked to a respective heavy chain by disulfide bonds in a “Y” shaped configuration. Proteolytic digestion of an antibody yields Fv (Fragment variable) and Fc (Fragment crystalline) domains. The antigen binding domains, Fab, include regions where the polypeptide sequence varies. The term F(ab′)₂ represents two Fab′ arms linked together by disulfide bonds. The central axis of the antibody is termed the Fc fragment. Each heavy chain has at one end a variable domain (V_{H}) followed by a number of constant domains (C_{H}). Each light chain has a variable domain (V_{L}) at one end and a constant domain (C_{L}) at its other end, the light chain variable domain being aligned with the variable domain of the heavy chain and the light chain constant domain being aligned with the first constant domain of the heavy chain (CH1). The variable domains of each pair of light and heavy chains form the antigen-binding site. The domains on the light and heavy chains have the same general structure and each domain comprises four framework regions, whose sequences are relatively conserved, joined by three hyper-variable domains known as complementarity determining regions (CDRs 1-3). These domains contribute specificity and affinity of the antigen-binding site. The isotype of the heavy chain (gamma, alpha, delta, epsilon or mu) determines immunoglobulin class (IgG, IgA, IgD, IgE or IgM, respectively). The light chain is either of two isotypes (kappa, κ or lambda, λ) found in all antibody classes.

The term “antibody” is used in the broadest sense and includes monoclonal antibodies (including full length or intact monoclonal antibodies), polyclonal antibodies, multivalent antibodies, multi-specific antibodies (e.g., bi-specific antibodies), and antibody fragments long enough to exhibit the desired biological activity.

The antibody according to the present invention is a molecule comprising at least the antigen-binding portion of an antibody. Antibody or antibodies according to the invention include intact antibodies, such as polyclonal antibodies or monoclonal antibodies (mAbs), as well as proteolytic fragments thereof, such as the Fab or F(ab′)₂ fragments. Single chain antibodies also fall within the scope of the present invention.

“Antibody fragments” comprise only a portion of an intact antibody, generally including an antigen binding site of the intact antibody and thus retaining the ability to bind antigen. Examples of antibody fragments encompassed by the present definition include: (i) the Fab fragment, having VL, CL, VH and CH1 domains; (ii) the Fab′ fragment, which is a Fab fragment having one or more cysteine residues at the C-terminus of the CH1 domain; (iii) the Fd fragment having VH and CH1 domains; (iv) the Fd′ fragment having VH and CH1 domains and one or more cysteine residues at the C-terminus of the CH1 domain; (v) the Fv fragment having the VL and VH domains of a single arm of an antibody; (vi) the dAb fragment (Ward et al., Nature 1989, 341, 544-546) which consists of a VH domain; (vii) isolated CDR regions; (viii) F(ab′)₂ fragments, a bivalent fragment including two Fab′ fragments linked by a disulphide bridge at the hinge region; (ix) single chain antibody molecules (e.g. single chain Fv; scFv) (Bird et al., Science 1988, 242, 423-426; and Huston et al., PNAS (USA) 1988, 85,5879-5883); (x) “diabodies” with two antigen binding sites, comprising a heavy chain variable domain (VH) connected to a light chain variable domain (VL) in the same polypeptide chain (see, e.g., EP 404,097; WO 93/11161; and Hollinger et al., Proc. Natl. Acad. Sci. USA, 1993, 90, 6444-6448); (xi) “linear antibodies” comprising a pair of tandem Fd segments (VH-CH1-VH-CH1) which, together with complementary light chain polypeptides, form a pair of antigen binding regions (Zapata et al. Protein Eng., 1995, 8, 1057-1062; and U.S. Pat. No. 5,641,870).

Single chain antibodies can be single chain composite polypeptides having antigen binding capabilities and comprising amino acid sequences homologous or analogous to the variable regions of an immunoglobulin light and heavy chain i.e. linked V_{H}-V_{L} or single chain Fv (scFv).

The term “neutralizing antibody” as used herein refers to a molecule having an antigen-binding site to a specific receptor or ligand target capable of reducing or inhibiting (blocking) activity or signaling through a receptor, as determined by in-vivo or in-vitro assays, as per the specification.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each monoclonal antibody is directed against a single determinant on the antigen. The modifier “monoclonal” is not to be construed as requiring production of the antibody by any particular method. Monoclonal Abs may be obtained by methods known to those skilled in the art. For example, the monoclonal antibodies to be used in accordance with the present invention may be made by the hybridoma method first described by Kohler et al., Nature 1975, 256, 495, or may be made by recombinant DNA methods (see, e.g., U.S. Pat. No. 4,816,567). The “monoclonal antibodies” may also be isolated from phage antibody libraries using the techniques described in Clackson et al., Nature 1991, 352, 624-628 or Marks et al., J. Mol. Biol., 1991, 222:581-597, for example.

The mAbs of the present invention may be of any immunoglobulin class including IgG, IgM, IgE, or IgA. A hybridoma producing a mAb may be cultivated in-vitro or in-vivo. High titers of mAbs can be obtained by in-vivo production where cells from the individual hybridomas are injected intra-peritoneally into pristine-primed Balb/c mice to produce ascites fluid containing high concentrations of the desired mAbs. Monoclonal Abs of isotype IgM or IgG may be purified from such ascites fluids, or from culture supernatants, using column chromatography methods well known to those of skill in the art.

The term “human antibody” as used herein refers to an antibody which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art.

The terms “molecule having the antigen-binding portion of an antibody” and “antigen-binding-fragments” as used herein is intended to include not only intact immunoglobulin molecules of any isotype and generated by any animal cell line or microorganism, but also the antigen-binding reactive fraction thereof, including, but not limited to, the Fab fragment, the Fab′ fragment, the F(ab′)₂ fragment, the variable portion of the heavy and/or light chains thereof, Fab mini-antibodies (see WO 93/15210, U.S. patent application Ser. No. 08/256,790, WO 96/13583, U.S. patent application Ser. No. 08/817,788, WO 96/37621, US patent application 08/999,554, the entire contents of which are incorporated herein by reference), dimeric bispecific mini-antibodies (see Muller et al., 1998) and single-chain antibodies incorporating such reactive fraction, as well as any other type of molecule in which such antibody reactive fraction has been physically inserted. Such molecules may be provided by any known technique, including, but not limited to, enzymatic cleavage, peptide synthesis or recombinant techniques.

The invention also provides conservative amino acid variants of the antibody molecules according to the invention. Variants according to the invention also may be made that conserve the overall molecular structure of the encoded proteins. Given the properties of the individual amino acids comprising the disclosed protein products, some rational substitutions will be recognized by the skilled worker. Amino acid substitutions, i.e. “conservative substitutions,” may be made, for instance, on the basis of similarity in polarity, charge, solubility, hydrophobicity, hydrophilicity, and/or the amphipathic nature of the residues involved. The term “antibody analog” as used herein refers to an antibody derived from another antibody by one or more conservative amino acid substitutions.

The term “antibody variant” as used herein refers to any molecule comprising the antibody of the present invention. For example, fusion proteins in which the antibody or an antigen-binding-fragment thereof is linked to another chemical entity is considered an antibody variant.

The term “non-fully-humanized monoclonal antibody” as used herein refers to a monoclonal antibody, having a heavy chain and/or a light chain variable domains in which the amino-acid sequences flanking and/or immediately adjacent to the CDRs are not fully human, i.e. are not identical to any known homologous or corresponding sequences taken from natural human antibodies.

The present invention is based in part on the results of a clinical trial conducted to evaluate the safety and efficacy of the anti CEACAM1 mAb CM-24 (NCT04731467). Part A of the trial assessed the safety of CM-24 administration and involved administration of CM-24 in combination with the anti-PD1 mAb Nivolumab, in dose escalation, to patients with solid tumors. Part C also assessed safety, and consisted of two subparts—part C1, during which 8 pancreatic cancer patients were administered CM-24 in combination with Nivolumab, and an additional therapeutic agent selected from gemcitabine and nab-paclitaxel; and part C2, in which 8 additional pancreatic cancer patients were administered CM-24 in combination with Nivolumab, and the chemotherapeutic cocktail Nal-IRI/5FU/LV. Part D, the final stage of the clinical trial, assesses the efficacy of the treatment, by monitoring the progression of the patients following treatments described above, compared to control patients who were administered standard therapy (gemcitabine/nab-paclitaxel or Nal-IRI/5FU/LV) and did not receive antibody therapy, in second line pancreatic ductal adenocarcinoma (PDAC).

The term “second-line” as used herein refers to a subsequent therapeutic regimen following an unsuccessful or insufficient initial therapeutic regimen. Second-line therapy can comprise the same therapeutic agents administered to the patient the first time, or entirely different ones.

The terms tumor-associated immune cells refer to the myeloid and lymphoid immune subsets in the tumor and the tumor microenvironment. In some embodiments, the tumor-associated immune cell is a lymphocyte cell selected from the group consisting of a tumor-infiltrating-lymphocyte (TIL) cell, a lymphokine-activated killer (LAK) cell, a cytokine induced killer (CIK) cell, a T cell, a B cell, an NK cell, and any combination thereof.

Tumor-associated lymphocytes and tumor infiltrating lymphocytes (TILs) are exchangeable and used to specify lymphocytes in the tumor and the tumor microenvironment.

In pharmaceutical and medicament formulations, the active agent is preferably utilized together with one or more pharmaceutically acceptable carrier(s) and optionally any other therapeutic ingredients. The carrier(s) must be pharmaceutically acceptable in the sense of being compatible with the other ingredients of the formulation and not unduly deleterious to the recipient thereof. The active agent is provided in an amount effective to achieve the desired pharmacological effect, as described above, and in a quantity appropriate to achieve the desired daily dose.

Typically, the molecules of the present invention comprising the antigen binding portion of an antibody or comprising another polypeptide including a peptide-mimetic will be suspended in a sterile saline solution for therapeutic uses. The pharmaceutical compositions may alternatively be formulated to control release of active ingredient (molecule comprising the antigen binding portion of an antibody) or to prolong its presence in a patient's system. Numerous suitable drug delivery systems are known and include, e.g., implantable drug release systems, hydrogels, hydroxymethylcellulose, microcapsules, liposomes, microemulsions, microspheres, and the like. Controlled release preparations can be prepared through the use of polymers to complex or adsorb the molecule according to the present invention. For example, biocompatible polymers include matrices of poly(ethylene-co-vinyl acetate) and matrices of a polyanhydride copolymer of a stearic acid dimer and sebaric acid. The rate of release of the molecule according to the present invention, i.e., of an antibody or antibody fragment, from such a matrix depends upon the molecular weight of the molecule, the amount of the molecule within the matrix, and the size of dispersed particles.

The pharmaceutical composition of this invention may be administered by any suitable means, such as orally, topically, intranasally, subcutaneously, intramuscularly, intravenously, intra-arterially, intraarticulary, intralesionally or parenterally. Ordinarily, intravenous (i.v.), administration is used.

It will be apparent to those of ordinary skill in the art that the therapeutically effective amount of the molecule according to the present invention will depend, inter alia upon the administration schedule, the unit dose of molecule administered, whether the molecule is administered in combination with other therapeutic agents, the immune status and health of the patient, the therapeutic activity of the molecule administered and the judgment of the treating physician. As used herein, a “therapeutically effective amount” refers to the amount of a molecule required to alleviate one or more symptoms associated with a disorder being treated over a period of time.

The term “therapeutically effective amount” refers to an amount of a drug effective to treat a disease or disorder in a mammal. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), the response rates (RR), duration of response, and/or quality of life.

The term “sugar” refers to monosaccharides, disaccharides, and polysaccharides, Examples of sugars include, but are not limited to, sucrose, trehalose, dextrose, and others.

The molecules of the present invention as active ingredients are dissolved, dispersed or admixed in an excipient that is pharmaceutically acceptable and compatible with the active ingredient as is well known. Suitable excipients are, for example, water, saline, phosphate buffered saline (PBS), dextrose, glycerol, ethanol, or the like and combinations thereof. Other suitable carriers are well known to those skilled in the art. In addition, if desired, the composition can contain minor amounts of auxiliary substances such as wetting or emulsifying agents, pH buffering agents.

The pharmaceutical composition according to the present invention may be administered together with an anti-neoplastic composition. According to a specific embodiment the anti-neoplastic composition comprises at least one chemotherapeutic agent. The chemotherapy agent, which could be administered together with the antibody according to the present invention, or separately, may comprise any such agent known in the art exhibiting anticancer activity, including but not limited to: mitoxantrone, topoisomerase inhibitors, spindle poison vincas: vinblastine, vincristine, vinorelbine (taxol), paclitaxel, docetaxel; alkylating agents: mechlorethamine, chlorambucil, cyclophosphamide, melphalan, ifosfamide; methotrexate; 6-mercaptopurine; 5-fluorouracil, cytarabine, gemcitabin; podophyllotoxins: etoposide, irinotecan, topotecan, dacarbazin; antibiotics: doxorubicin (adriamycin), bleomycin, mitomycin; nitrosoureas: carmustine (BCNU), lomustine, epirubicin, idarubicin, daunorubicin; inorganic ions: cisplatin, carboplatin; interferon, asparaginase; hormones: tamoxifen, leuprolide, flutamide, and megestrol acetate.

According to a specific embodiment, the chemotherapeutic agent is selected from the group consisting of alkylating agents, antimetabolites, folic acid analogs, pyrimidine analogs, purine analogs and related inhibitors, vinca alkaloids, epipodophyllotoxins, antibiotics, L-asparaginase, topoisomerase inhibitor, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. According to another embodiment, the chemotherapeutic agent is selected from the group consisting of 5-fluorouracil (5-FU), leucovorin (LV), irenotecan, oxaliplatin, capecitabine, paclitaxel and doxetaxel. Two or more chemotherapeutic agents can be used in a cocktail to be administered in combination with administration of the anti-CEACAM1 antibody.

The term “treatment” as used herein refers to both therapeutic treatment and prophylactic or preventative measures. Those in need of treatment include those already with the disorder as well as those in which the disorder is to be prevented.

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia. More particular examples of such cancers include melanoma, lung, thyroid, breast, colon, prostate, hepatic, bladder, renal, cervical, pancreatic, leukemia, lymphoma, myeloid, ovarian, uterus, sarcoma, biliary, or endometrial cancer.

The term “anti-neoplastic composition” refers to a composition useful in treating cancer comprising at least one active therapeutic agent capable of inhibiting or preventing tumor growth or function, and/or causing destruction of tumor cells. Therapeutic agents suitable in an anti-neoplastic composition for treating cancer include, but not limited to, chemotherapeutic agents, radioactive isotopes, toxins, cytokines such as interferons, and antagonistic agents targeting cytokines, cytokine receptors or antigens associated with tumor cells. Preferably the therapeutic agent is a chemotherapeutic agent.

The term “diagnosing” as used herein refers to determining presence or absence of a pathology, classifying a pathology or a symptom, determining a severity of the pathology, monitoring pathology progression, forecasting an outcome of a pathology and/or prospects of recovery.

The term “respond therapeutically” as used herein denotes the capacity of a subject to exhibit positive and beneficial reactions to a specified therapeutic intervention or treatment. In the context of the present invention, “responding therapeutically” encompasses a range of favorable physiological, biochemical, or clinical responses that contribute to the amelioration, alleviation, or prolonged life expectancy in a [cancer] subject. Such therapeutic responses may manifest as improved symptoms, enhanced overall well-being, normalized physiological parameters, or other measurable indicators of positive treatment outcomes.

The term “amino-acid residue mutation” as used herein refers to a substitution, an insertion, or a deletion of a single amino-acid residue. The term “amino-acid residue back-mutation” as used herein refers to a substitution of a single amino-acid residue found in a human antibody framework to a corresponding amino-acid residue found in a murine antibody framework.

The following examples are presented in order to more fully illustrate some embodiments of the invention. They should, in no way be construed, however, as limiting the broad scope of the invention.

### EXAMPLES

TABLE 1
CDR sequences. A humanized mAb according
to the present invention
comprises the following six CDRs:

| V_{L} CDR3 | V_{L} CDR2 | V_{L} CDR1 | V_{H} CDR3 | V_{H} CDR2 | V_{H} CDR1 |
|---|---|---|---|---|---|
| QQGKSLP | YTSRLHS | RTSQDIG | GDYYGGF | VINPGSG | GYAFTNN |
| RTEKFKG | SEQ ID | NYLN | AVDY | DTNYN | LIE |
| SEQ ID | NO: 5 | SEQ ID | SEQ ID | SEQ ID | SEQ ID |
| NO: 6 | | NO: 4 | NO: 3 | NO: 2 | NO: 1 |

TABLE 2
Non-CDR sequences of fully murine and fully human
variable regions.

| CDR3-X | CDR2-X-CDR3 | CDR1-X-CDR2 | X-CDR1 | Chain |
|---|---|---|---|---|
| WGQGTSVTVSS | KATLTADKSSNTAYM | WVKQRPGQGLEW | QVQLQQSGAELVR | Murine |
| (SEQ ID | QLSSLTSDDSAVYFC | IG (SEQ ID | PGTSVKVSCKAS | H |
| NO: 39) | AR (SEQ ID NO: 38) | NO: 37) | (SEQ ID NO: 36) | |
| WGQGTTVTVSS | RVTMTRDTSISTAYM | WVRQAPGQGLEW | QVQLVQSGAEVK | Human |
| (SEQ ID | ELSRLRSDDTAVYYC | MG (SEQ ID | KPGASVKVSCKAS | H |
| NO: 10) | AR (SEQ ID NO: 9) | NO: 8) | (SEQ ID NO: 7) | |
| FGGGTKLEIK | GVPSRFSGSGSGTDYS | WYQQKPDGTVKL | DIQMTQTTSSLSAS | Murine |
| (SEQ ID | LTISNLEQEDIATYFC | LIY (SEQ ID | LGDRVTISC (SEQ | L |
| NO: 43) | (SEQ ID NO: 42) | NO: 41) | ID NO: 40) | |
| FGGGTKVEIK | GVPSRFSGSGSGTDFT | WYQQKPGKAPKL | DIQMTQSPSSLSAS | Human |
| (SEQ ID | FTISSLQPEDIATYYC | LIY (SEQ ID | VGDRVTITC (SEQ | L |
| NO: 14) | (SEQ ID NO: 13) | NO: 12) | ID NO: 11) | |

TABLE 3
Non-CDR sequences of humanized back-mutated
heavy chain variable regions.

| CDR3-X | CDR2-X-CDR3 | CDR1-X-CDR2 | X-CDR1 | Variant |
|---|---|---|---|---|
| WGQGTSVTVSS | RATLTADKSINTAYME | WVKQAPGQGLEW | QVQLVQSGAELKKP | VH1 |
| (SEQ ID | LSSLTSDDSAVYFCAR | IG (SEQ ID | GASVKVSCKAS | |
| NO: 23) | (SEQ ID NO: 18) | NO: 16) | (SEQ ID NO: 15) | |
| WGQGTTVTVSS | RATLTADKSINTAYME | WVKQAPGQGLEW | QVQLVQSGAEVKK | VH2 |
| (SEQ ID | LSRLRSDDTAVYFCAR | IG (SEQ ID | PGASVKVSCKAS | |
| NO: 10) | (SEQ ID NO: 19) | NO: 16) | (SEQ ID NO: 7) | |
| WGQGTTVTVSS | RATLTADKSINTAYME | WVRQAPGQGLEW | QVQLVQSGAEVKK | VH3 |
| (SEQ ID | LSRLRSDDTAVYYCAR | IG (SEQ ID | PGASVKVSCKAS | |
| NO: 10) | (SEQ ID NO: 20) | NO: 17) | (SEQ ID NO: 7) | |
| WGQGTTVTVSS | RATLTADKSISTAYME | WVRQAPGQGLEW | QVQLVQSGAEVKK | VH4 |
| (SEQ ID | LSRLRSDDTAVYYCAR | IG (SEQ ID | PGASVKVSCKAS | |
| NO: 10) | (SEQ ID NO: 21) | NO: 17) | (SEQ ID NO: 7) | |
| WGQGTTVTVSS | RVTMTADKSISTAYME | WVRQAPGQGLEW | QVQLVQSGAEVKK | VH5 |
| (SEQ ID | LSRLRSDDTAVYYCAR | IG (SEQ ID | PGASVKVSCKAS | |
| NO: 10) | (SEQ ID NO: 22) | NO: 17) | (SEQ ID NO: 7) | |

* Bold and underlined amino-acids with back mutations to murine sequence.

TABLE 4
Non-CDR sequences of back-mutated
humanized light chain variable regions.

| CDR3-X | CDR2-X-CDR3 | CDR1-X-CDR2 | X-CDR1 | Variant |
|---|---|---|---|---|
| FGGGTKVEIK | GVPSRFSGSGSGTDYT | WYQQKPGKAVKL | DIQMTQSPSSLS | VL1 |
| (SEQ ID | LTISSLQQEDIATYFC | LIY (SEQ ID | ASVGDRVTITC | |
| NO: 14) | (SEQ ID NO: 25) | NO: 24) | (SEQ ID NO: 11) | |
| FGGGTKVEIK | GVPSRFSGSGSGTDYT | WYQQKPGKAVKL | DIQMTQSPSSLS | VL2 |
| (SEQ ID | LTISSLQPEDIATYFC | LIY (SEQ ID | ASVGDRVTITC | |
| NO: 14) | (SEQ ID NO: 26) | NO: 24) | (SEQ ID NO: 11) | |
| FGGGTKVEIK | GVPSRFSGSGSGTDYT | WYQQKPGKAVKL | DIQMTQSPSSLS | VL3 |
| (SEQ ID | FTISSLQPEDIATYFC | LIY (SEQ ID | ASVGDRVTITC | |
| NO: 14) | (SEQ ID NO: 27) | NO: 24) | (SEQ ID NO: 11) | |

* Bold and underlined amino-acids with back mutations to murine sequence.

TABLE 5
Non-CDR sequences of back-mutated humanized variable
regions.

| SEQ | | |
|---|---|---|
| ID | | |
| NO: | Amino acid sequence | Variable regions |
| 28 | SEQ ID NO: 15-SEQ ID NO: 1- | Heavy chain |
| | SEQ ID NO: 16-SEQ ID NO: 2-SEQ ID | variable region #1 |
| | NO: 18-SEQ ID NO: 3-SEQ ID NO: 23 | |
| 29 | SEQ ID NO: 7-SEQ ID NO: 1- | Heavy chain |
| | SEQ ID NO: 16-SEQ ID NO: 2-SEQ ID | variable region #2 |
| | NO: 19-SEQ ID NO: 3-SEQ ID NO: 10 | |
| 30 | SEQ ID NO: 7-SEQ ID NO: 1- | Heavy chain |
| | SEQ ID NO: 17-SEQ ID NO: 2-SEQ ID | variable region #3 |
| | NO: 20-SEQ ID NO: 3-SEQ ID NO: 10 | |
| 31 | SEQ ID NO: 7-SEQ ID NO: 1- | Heavy chain |
| | SEQ ID NO: 17-SEQ ID NO: 2-SEQ ID | variable region #4 |
| | NO: 21-SEQ ID NO: 3-SEQ ID NO: 10 | |
| 32 | SEQ ID NO: 7-SEQ ID NO: 1- | Heavy chain |
| | SEQ ID NO: 17-SEQ ID NO: 2-SEQ ID | variable region #5 |
| | NO: 22-SEQ ID NO: 3-SEQ ID NO: 10 | |
| 33 | SEQ ID NO: 11-SEQ ID NO: 4- | Light chain |
| | SEQ ID NO: 24-SEQ ID NO: 5-SEQ ID | variable region #1 |
| | NO: 25-SEQ ID NO: 6-SEQ ID NO: 14 | |
| 34 | SEQ ID NO: 11-SEQ ID NO: 4- | Light chain |
| | SEQ ID NO: 24-SEQ ID NO: 5-SEQ ID | variable region #2 |
| | NO: 26-SEQ ID NO: 6-SEQ ID NO: 14 | |
| 35 | SEQ ID NO: 11-SEQ ID NO: 4- | Light chain |
| | SEQ ID NO: 24-SEQ ID NO: 5-SEQ ID | variable region #3 |
| | NO: 27-SEQ ID NO: 6-SEQ ID NO: 14 | |

The reference fully-humanized heavy chain sequence (SEQ ID NO: 57), to which back-mutations are introduced is composed of:

(SEQ ID NO: 7)
QVQLVQSGAEVKKPGASVKVSCKAS-
(SEQ ID NO: 1)
GYAFTNNLIE-
(SEQ ID NO: 8)
WVRQAPGQGLEWMG-
(SEQ ID NO: 2)
VINPGSGDTNYNEKFKG-
(SEQ ID NO: 9)
RVTMTRDTSISTAYMELSRLRSDDTAVYYCAR-
(SEQ ID NO: 3)
GDYYGGFAVDY-
(SEQ ID NO: 10)
WGQGTTVTVSS.

The reference fully-humanized light chain sequence (SEQ ID NO: 58), to which back-mutations are introduced is composed of:

(SEQ ID NO: 11)
DIQMTQSPSSLSASVGDRVTITC-
(SEQ ID NO: 4)
RTSQDIGNYLN-
(SEQ ID NO: 12)
WYQQKPGKAPKLLIY-
(SEQ ID NO: 5)
YTSRLHS-
(SEQ ID NO: 13)
GVPSRFSGSGSGTDFTFTISSLQPEDIATYYC-
(SEQ ID NO: 6)
QQGKSLPRT-
(SEQ ID NO: 14)
FGGGTKVEIK

### Example 1. Design of CDR-Grafted Antibody Variable Region Sequences

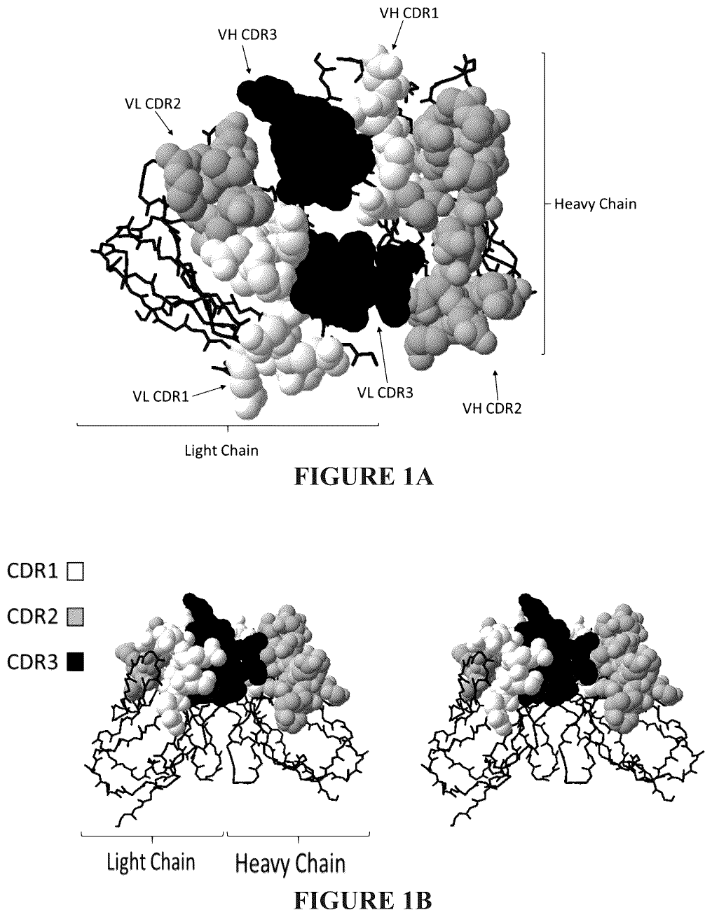

Structural models of the chimeric anti-CEACAM-1 antibody V regions were produced using Swiss PDB and analyzed in order to identify amino acids in the V region frameworks that may be important for the binding properties of the antibody (FIG. 1). These amino acids were noted for incorporation into one or more variant CDR-grafted antibodies. Both the V_{H} and V_{κ} sequences of CM-10 contain typical framework residues and the CDR 1, 2 and 3 motifs are comparable to many murine antibodies. The CDRs were taken directly from the murine sequence. The Swiss PDB models were then analyzed, together with mouse/human homologies at critical positions, to highlight framework regions and individual residues which could potentially impact on the presentation of the CDRs.

### Design of Variants

The heavy and light chain V region amino acid sequence were compared against a database of human germline V region sequences in order to identify the heavy and light chain human sequences with the greatest degree of homology for use as V region frameworks. A series of heavy and light chain V regions were then designed by grafting the CDRs onto the frameworks and, if necessary, by back-mutation to the murine sequence of residues identified above as potentially critical to the restoration of the chimeric antibody binding efficiency. It was considered that a small number of mouse residues needed to be retained in each variant. Variant sequences with lowest incidence of potential T cell epitopes were then selected as determined by application of Antitope's proprietary in-silico technologies, iTope™ and TCED™ (T Cell Epitope Database) (Perry et al 2008, Bryson et al 2010). The number of back mutations was determined by the starting murine sequence. From the structural analysis, a maximum of 13 positions were identified in the V_{H} and 5 positions were identified in the V_{κ} as residues which could be structurally important. These were prioritized and variants were designed which incorporated varying numbers of these. Although there is no theoretical limit to the number of back mutations, the more back mutations incorporated, the less human the sequence may be. Five V_{H} chains and three V_{κ} chains were designed with sequences set forth in SEQ ID NOs: 28 to 35.

iTope™ and TCED™

The iTope™ software predicts favorable interactions between amino acid side chains of a peptide and specific binding pockets (in particular pocket positions; p1, p4, p6, p7 and p9) within the binding grooves of 34 human MHC class II alleles. The location of key binding residues is achieved by the in silico generation of 9mer peptides that overlap by one amino acid spanning the test protein sequence. In-house comparisons with physical MHC class II binding experiments has shown that iTope™ can be used to successfully discriminate with high accuracy between peptides that either bind or do not bind MHC class II molecules. However, the results should be assessed in the light of the fact that all predictive methods for MHC class II binding inherently over-predict the number of T cell epitopes since they do not allow for other important processes during antigen presentation such as protein/peptide processing, recognition by the T cell receptor or T cell tolerance to the peptide.

The TCED™ contains the sequences of all the peptides previously screened in EpiScreen™ T cell epitope mapping assays. The TCED™ is used to search any test sequence against a large (>10,000 peptides) database of peptides by BLAST search in order to specifically select segments that had previously been shown not to stimulate T cell responses. In addition, any regions with significant homology to T cell epitopes in the database were discarded.

### Construction of CDR-Grafted Variants

All variant CDR-grafted V_{H} and V_{κ} region genes for CM-10 were synthesized using a series of overlapping oligonucleotides that were annealed, ligated and PCR amplified to give full length synthetic V regions. The assembled variants were then cloned directly into an expression vector system for both IgG1 and IgG4 (S241P) V_{H} chains and V_{κ} chains. All constructs were confirmed by sequencing.

### Construction, Expression and Purification of Antibodies

The chimeric antibody genes and all combinations of CDR-grafted V_{H} and V_{κ} chains (i.e. a total of 15 pairings for each of IgG1 and IgG4 (S241P)) were transiently transfected into HEK293-EBNA (ATCC cat. no. CRL-10852) cells using calcium phosphate. The transient transfections were incubated for up to five days prior to harvesting supernatants.

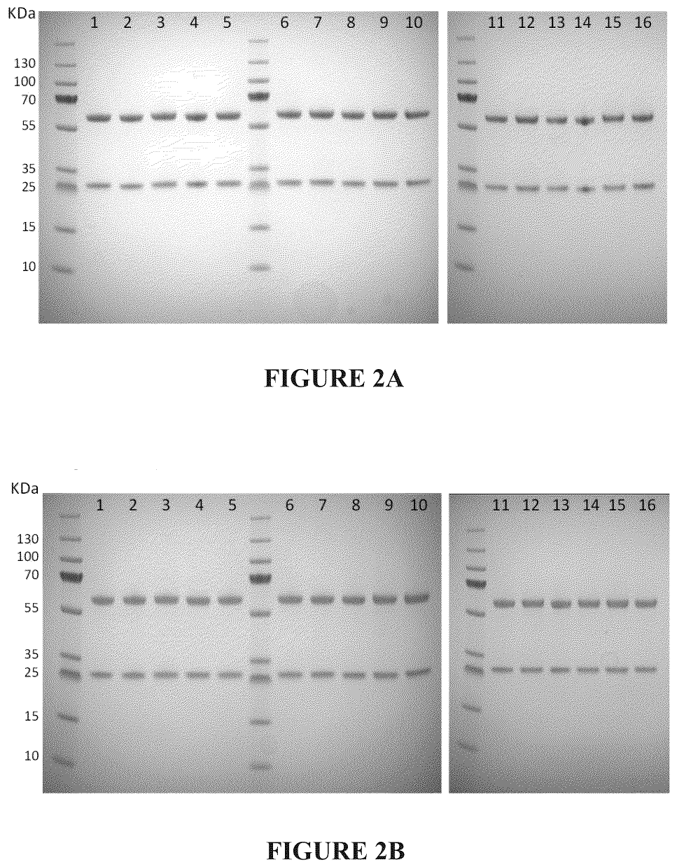

The chimeric antibodies and CDR-grafted variants of CM-10 were purified from transient cell culture supernatants on a Protein A sepharose column (GE Healthcare cat. no. 110034-93), buffer exchanged into 1X PBS pH 7.4 and quantified by OD_{280 nm} using an extinction coefficient based on the predicted amino acid sequence (E_{c(}0.1%)=˜1.37-1.40 for CM-10 chimeric antibody and variants). The chimeric antibodies and lead humanized variants were analyzed by reducing SDS-PAGE. Bands corresponding to the predicted sizes of the V_{H} and V_{κ} chains were observed with no evidence of any contamination (FIG. 2).

### Example 2. Competition Binding of Purified Antibodies to Human CEACAM-1

The binding of purified chimeric CM-10 antibody together with the chimeric antibodies and each of the CDR-grafted variants to recombinant human CEACAM-1 were assessed in a competitive ELISA. A dilution series (three-fold) of chimeric or humanized antibodies from 20 μg/ml to 0.009 μg/ml was premixed with a constant concentration of biotinylated chimeric CM-10 (0.005 μg/ml, final concentration) before incubating for 1 hour at room temperature on a Nunc Immuno MaxiSorp 96 well flat bottom microtitre plate (Fisher cat. no. DIS-971-030J) pre-coated with 0.5 μg/ml recombinant human CEACAM-1 (R&D Systems cat. no. 2244-CM-050) diluted in 1X PBS pH 7.4. The binding of the biotinylated antibody was detected with streptavidin-HRP (Sigma cat. no. S5512) and TMB substrate (Invitrogen cat. no. 00-2023). The reaction was stopped with 3M HC1, absorbance read at 450 nm on a Dynex Technologies MRX TC II plate reader and the binding curves plotted.

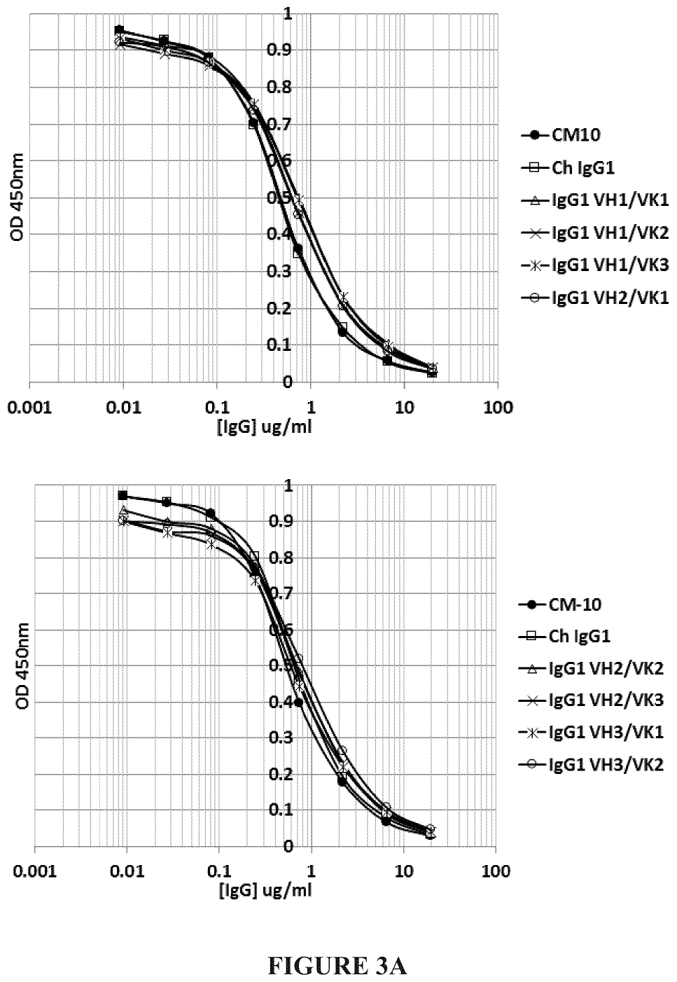

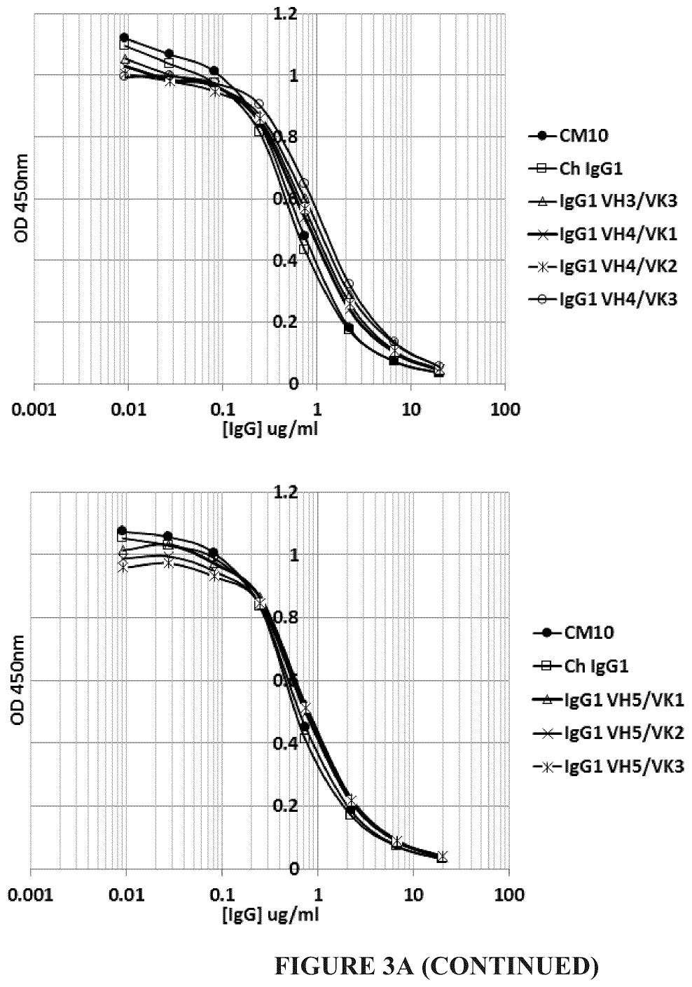

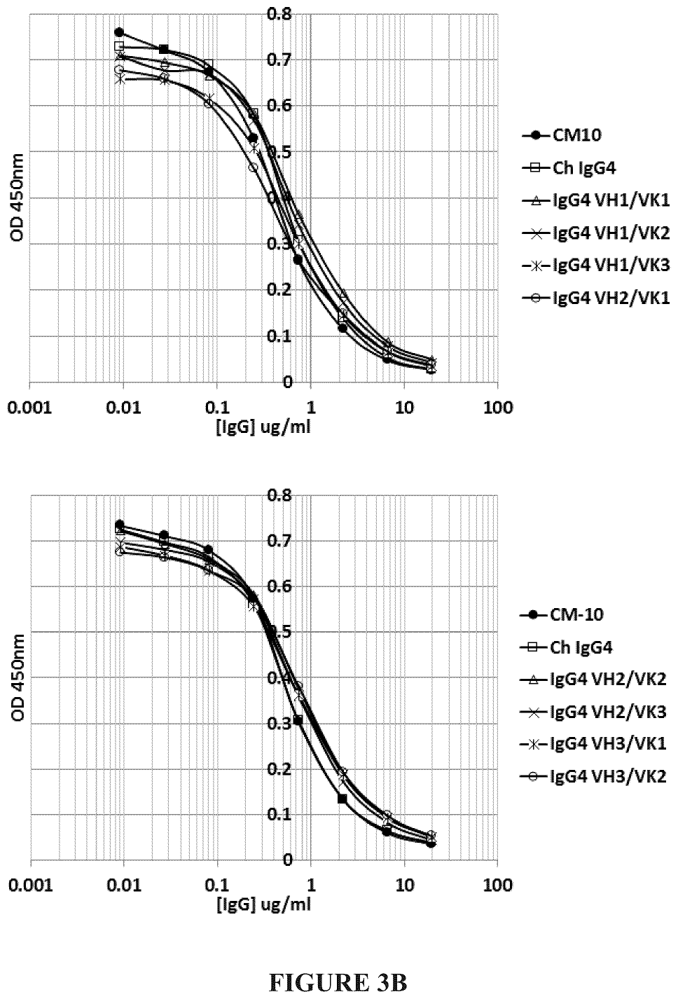

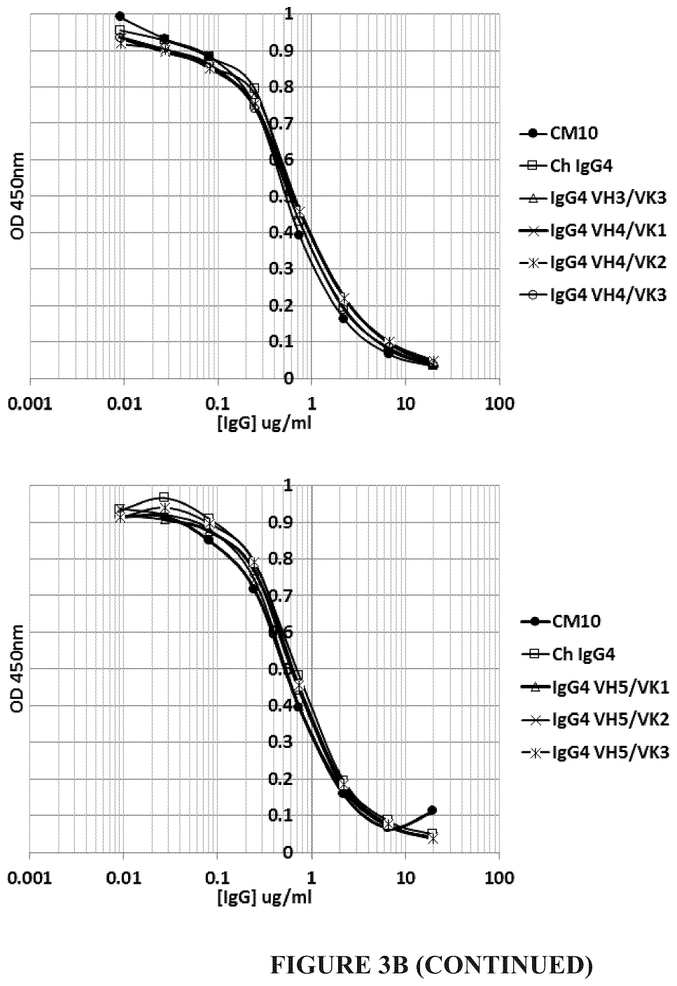

All humanized variants gave similar binding profiles to chimeric CM-10 with the binding curves shown in FIG. 3. These data were used to calculate IC₅₀ values for each antibody and was normalized to the IC₅₀ of chimeric CM-10 as included on each ELISA and as shown in Tables 6 and 7.

TABLE 6
IC₅₀ values for humanized anti-CEACAM-1 variants.

| | IC₅₀ [IgG] μg/mL | |
|---|---|---|
| Construct | IgG1 Variants | IgG4 (S241P) Variants |
| VH1/VK1 | 0.78 | 0.78 |
| VH1/VK2 | 0.70 | 0.70 |
| VH1/VK3 | 0.77 | 0.54 |
| VH2/VK1 | 0.68 | 0.43 |
| VH2/VK2 | 0.78 | 0.76 |
| VH2/VK3 | 0.76 | 0.71 |
| VH3/VK1 | 0.69 | 0.71 |
| VH3/VK2 | 0.85 | 0.77 |
| VH3/VK3 | 0.86 | 0.73 |
| VH4/VK1 | 0.73 | 0.69 |
| VH4/VK2 | 0.78 | 0.69 |
| VH4/VK3 | 0.99 | 0.63 |
| VH5/VK1 | 0.77 | 0.74 |
| VH5/VK2 | 0.72 | 0.70 |
| VH5/VK3 | 0.74 | 0.70 |

TABLE 7
Calculated relative IC₅₀ values for
humanized anti-CEACAM-1 variants.

| | IC₅₀ normalized to CM-10 | |
|---|---|---|
| Construct | IgG1 Variants | IgG4 Variants |
| VH1/VK1 | 1.46 | 1.52 |
| VH1/VK2 | 1.32 | 1.36 |
| VH1/VK3 | 1.44 | 1.05 |
| VH2/VK1 | 1.29 | 0.84 |
| VH2/VK2 | 1.24 | 1.26 |
| VH2/VK3 | 1.21 | 1.16 |
| VH3/VK1 | 1.10 | 1.16 |
| VH3/VK2 | 1.36 | 1.26 |
| VH3/VK3 | 1.37 | 1.23 |
| VH4/VK1 | 1.15 | 1.15 |
| VH4/VK2 | 1.24 | 1.17 |
| VH4/VK3 | 1.56 | 1.06 |
| VH5/VK1 | 1.17 | 1.23 |
| VH5/VK2 | 1.09 | 1.16 |
| VH5/VK3 | 1.12 | 1.17 |

The normalized IC₅₀ data showed a range of 0.84 to 1.56 for all variants tested indicating that the binding efficiencies of all CDR-grafted antibodies to human CEACAM-1 were comparable to that of the chimeric CM-10.

### Example 3. Combination of Humanized mAb to CEACAM1 and Anti PD-1/PD-L Antibodies

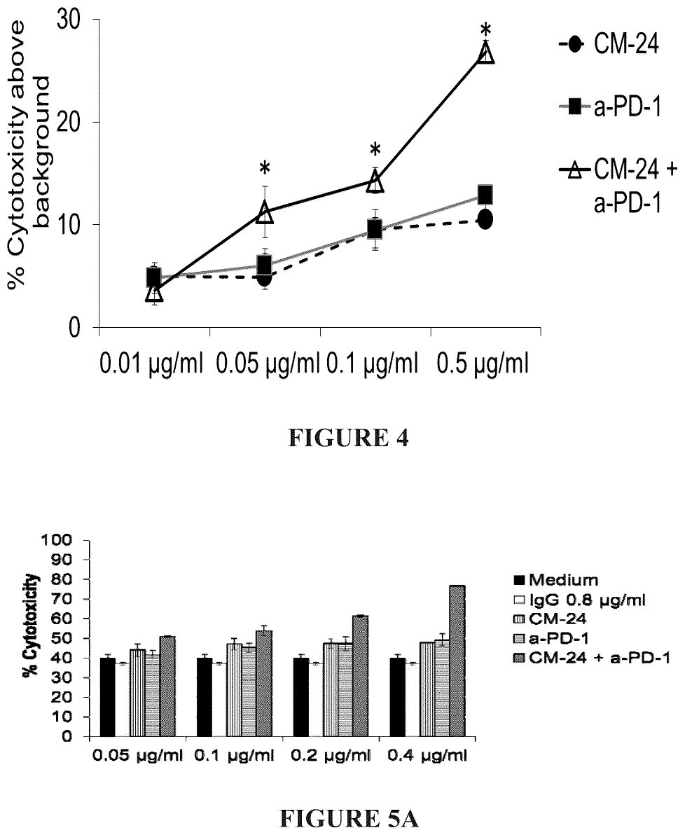

A. Synergistic effects of anti-CEACAM1 and anti-PD-1 antibodies on the cytotoxicity of human TIL cells against human melanoma cells was demonstrated. Human melanoma cancer cells (MALME 3M) were grown in the presence of IFN-ã to induce PD-L1 expression. Human TIL cells (TIL14) were incubated with a monoclonal antibody to human CEACAM1 (CM-24) (0.01 μg/ml, 0.05 μg/ml, 0.1 μg/ml, 0.5 μg/ml), a monoclonal antibody to human PD-1 (clone E12.2H7) or with a combination of both antibodies (0.005, 0.025, 0.05 and 0.25 μg/ml of each antibody) for 30 minutes at 37° C. IFN-ã-treated human melanoma cancer cells were added for overnight incubation, prior to cytotoxicity evaluation. FIG. 4 demonstrates that both anti-CEACAM1 antibodies and anti-PD-1 antibodies were able to bind their respective targets on human lymphocytes such as TIL cells, and that this binding significantly increased the toxicity of the human TIL cells against human cancer cells over each monotherapy alone. It was therefore concluded that protecting lymphocytes from immuno-suppressive signals from target cancer cells results in substantial cytotoxicity toward these cancer cells.

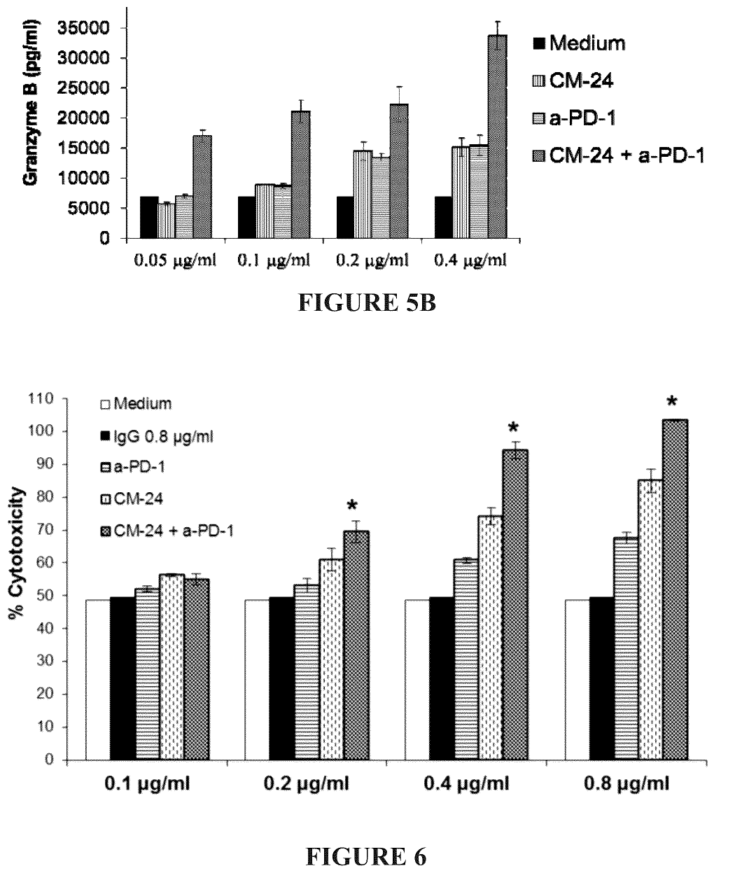

B. Synergistic effects of anti-CEACAM1 and anti-PD-1 antibodies on Granzyme B levels and the cytotoxicity of human TIL cells against human melanoma cells when anti-PD-1 antibodies are added prior to the addition of anti-CEACAM1 antibodies was also shown: Human melanoma cancer cells (MALME 3M) were grown in the presence of IFN-ã to induce PD-L1 expression. Human TIL cells (TIL14) were incubated with medium only (black), non-specific IgG antibody (0.8 μg/ml, white), various concentrations (0.05 μg/ml, 0.1 μg/ml, 0.2 μg/ml, 0.4 μg/ml, 0.8 μg/ml) of CM-24, a mAb to human PD-1 (clone E12.2H7) or a combination of both antibodies (0.05 μg/ml each, 0.1 μg/ml each, 0.2 μg/ml each, 0.4 μg/ml each, 0.8 μg/ml each). The monoclonal antibody to human PD-1 was added first for 30 minutes at 37° C., followed by the addition of the mAb to human CEACAM1. IFN-ã-treated human melanoma cancer cells were added for overnight incubation, prior to cytotoxicity evaluation. The combination index (CI) was calculated to be 0.15. In the same assay, the level of the cytotoxic protein granzyme B that is secreted upon cytotoxic cell activation was evaluated by commercial granzyme B ELISA Kit. FIG. 5 demonstrates that anti-CEACAM1 antibodies and anti-PD-1 antibodies are able to bind their respective targets on human lymphocytes such as TIL cells, and that this binding increases the granzyme B secretion and toxicity of the human TIL cells against human cancer cells. FIG. 5 indicates again that protecting lymphocytes from immuno-suppressive signals from target cancer cells results in substantial cytotoxicity toward target cancer cells and suggests that timing could be a critical factor in the combined therapy.

C. Synergistic effects of anti-CEACAM1 and anti-PD-L1 antibodies on Granzyme B levels and the cytotoxicity of human TIL cells against human melanoma cells when anti-PD-L1 antibodies are added prior to the addition of anti-CEACAM1 antibodies (data not shown). Human melanoma cells (MALME 3M) were grown in the presence of IFN-ã to induce PD-L1 expression. Human TIL cells (TIL14) were incubated with medium only (black), non-specific IgG antibody (0.8 μg/ml, white), various concentrations (0.05g/ml, 0.1 μg/ml, 0.2 μg/ml, 0.4 μg/ml, 0.8 μg/ml) of a monoclonal antibody to human CEACAM1 (CM-24), a monoclonal antibody to human PD-L1 (clone 29E.2A3) or a combination of both antibodies (0.05 μg/ml each, 0.1 μg/ml each, 0.2 μg/ml each, 0.4 μg/ml each, 0.8 μg/ml each). The anti-PD-L1 antibody was added first for 30 minutes at 37° C., followed by the addition of the monoclonal antibody to human CEACAM1. IFN-ã-treated human melanoma cancer cells were added for overnight incubation prior to cytotoxicity evaluation. The combination index (CI) was calculated to be 0.67. Results represent an average of % cytotoxicity ±SE as determined by classical LDH release assay from triplicate wells per treatment. * P≤0.05 paired T-test compared to a-PD-L1 only. In the same assay, the levels of the cytotoxic protein granzyme B that is secreted upon cytotoxic cell activation was evaluate by commercial granzyme B ELISA Kit. Results represent average granzyme B level from triplicate wells per treatment. The results demonstrate that anti-CEACAM1 antibodies and anti-PD-L1 antibodies are able to bind their respective targets on human lymphocytes (such as TIL cells) and on human cancer cells (such as melanoma cells), and that this binding increases the granzyme B secretion and toxicity of the human TIL cells against human cancer cells. It was further demonstrates that blocking the PD-1/PD-L1 and CEACAM1/CEACAM1 interactions can result in synergistic affect and that protecting lymphocytes from the PD-1^{lymphocyte}/PD-Ligand^{cancer cell} immuno-suppressive signal results in substantial cytotoxicity toward these cancer cells, regardless to the antigen targeted, either PD-1, PD-L1 or PD-L2.

D. Synergistic effects of anti-CEACAM1 and anti-PD-1 antibodies on the cytotoxicity of human LAK cells against human melanoma cells when anti-PD-1 antibodies are added prior to the addition of anti-CEACAM1 antibodies. Human melanoma cells (SKMEL28, CEACAM1 positive, PD-L1 positive) were grown in the presence of IFN-ã to induce PD-L1 expression. Human LAK (lymphokine-activated killer) cells generated by activation of PBMCs from a healthy human donor with IL-2 (500 units/ml) for 7 days were incubated with medium only (white), non-specific IgG antibody (0.8 μg/ml, black), various concentrations (0.1 μg/ml, 0.2 μg/ml, 0.4 μg/ml, 0.8 μg/ml) of a monoclonal antibody to human CEACAM1 (CM-24), a monoclonal antibody to human PD-1 (clone E12.2H7) or a combination of both antibodies (0.1 μg/ml each, 0.2 ug/ml each, 0.4 μg/ml each, 0.8 μg/ml each). The monoclonal antibody to human PD-1 was added first for 30 minutes at 37° C., followed by the addition of the monoclonal antibody to human CEACAM1. IFN-ã-treated human melanoma cells were added for 24 hour incubation, prior to cytotoxicity evaluation. FIG. 6 demonstrates that anti-CEACAM1 antibodies and anti-PD-1 antibodies are able to bind their respective targets on activated human lymphocytes such as LAK cells, and that this binding increases the toxicity of the human LAK cells against human cancer cells. The combination index (CI) was calculated to be <0.8. FIG. 6 further demonstrates that the binding of these antibodies to LAK cells is somehow interrelated, warranting a further study of their binding mechanism, and that this mechanism is present in variety of activated lymphocytes.

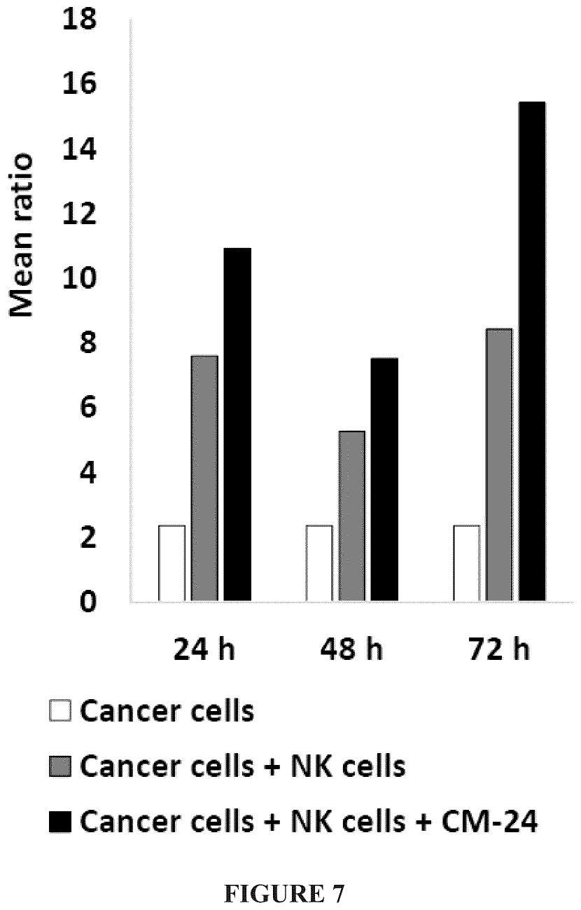

E. Treatment with anti-CEACAM1 antibodies increases PD-L1 expression on target cancer cells. Human NK cells (NK92MI) were incubated with or without a monoclonal antibody to human CEACAM1 (10 μg/ml CM-24), followed by the addition of human melanoma cancer cells (SKMEL28). The cells were incubated for 24, 48 and 72 hours and PD-L1 levels were measured at each time point by FACS analysis. The mean ratio levels of anti-PD-L1 compared to an appropriate isotype control for the indicated treatments at different time points is shown in FIG. 7 demonstrating that the expression of CEACAM1 and PD-L1 on cancer cells is indeed interrelated. The addition of anti-CEACAM1 antibodies results in increased PD-L1 expression on surviving cancer cells thus providing additional support for combined treatment with both agents. It may be beneficial to treat cancer by first administering anti CEACAM1 antibodies, and then further administering anti-PD-L1 and/or anti-PD-L2 antibodies, since the number of PD-L1 proteins on the cancer cells remains relatively high, making the cells more sensitive for anti PD-1/ PD-L1 antibodies treatment, implying that the combinational therapy may improve the clinical outcome. Administration of different antibodies at separate times, rather than concurrently, maximizes the cytotoxic effect of lymphocytes against cancer cells. Without being bound to any theory or mechanism, this finding may be linked to another surprising finding of the present invention, according to which treatment with anti-CEACAM1 antibodies increases PD-L1 expression on target cancer cells. Hypothetically, this would support the need for a plurality of antibodies to obtain improved efficacy for cytotoxic lymphocytes. It may be envisioned that the administration of anti-PD-1 antibodies first blocks PD-1 molecules on lymphocytes, the later administration of anti-CEACAM1 antibodies blocks CEACAM1 molecules on lymphocytes and/or target cancer cells and increases expression of PD-1 ligands on target cancer cells. However, since PD-1 molecules on lymphocytes are already blocked, the elevated expression levels of PD-1 ligands on target cancer cells do not prevent lymphocytes from efficiently exerting their full cytotoxic potential.

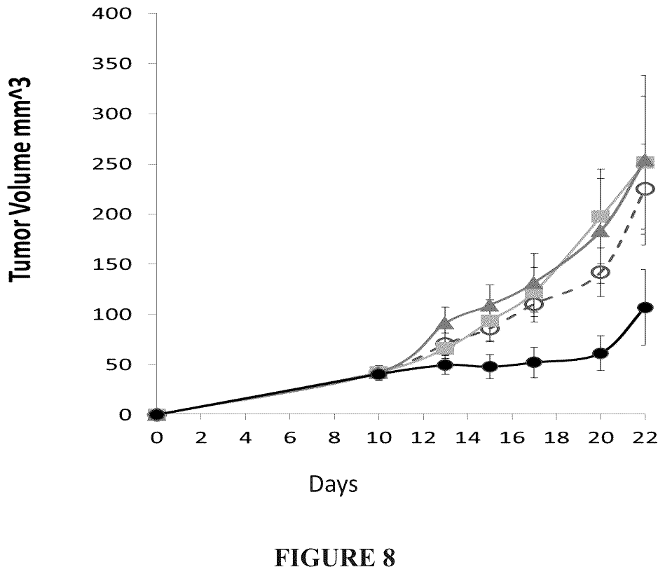

F. Synergistic effects of anti-CEACAM1 and anti-PD-1 antibodies on tumor progression in immuno-competent mice. Murine lymphoma cells (5*10⁶, A20) were allografted into the abdomen of Balb/C mice by sub-cutaneous injection on Day 1. On day 10, tumors reached an average volume of 45 mm³, and mice were randomized into 4 separate groups (11-12 mice per group), and intravenously administered with either PBS, CC-1 (anti murine CEACAM1 antibody, 6 mg/kg, PRM-1 (anti murine PD-1 antibody, 6 mg/kg) or a combination of CC-1 and PRM-1 (6 mg/kg each). Treatments were repeated on days 15 and 20. The effect of a monoclonal antibody to human CEACAM1 alone, a monoclonal antibody to human PD-1 alone, and a combination of both antibodies on tumor growth inhibition was followed. Immuno-competent Balb/C mice were selected for this experiment to allow evaluation of anti-murine-CEACAM1 and anti-murine-PD-1 antibodies' biological activity in mice with intact immune system and to evaluate the entire immune system reaction against the murine cancer cells. As a whole, this model simulates therapies in humans, in which cancer patients would receive combinations of anti-human-CEACAM1 anti-human-PD-1/PD-L1/PD-L2 antibodies. Without being bound to any theory or mechanism, it is hypothesized that a combination of anti-CEACAM1 and anti- PD-1/PD-L1/PD-L2 antibodies would prohibit cancer cells to circumvent the activation and cytotoxicity of the patient's immune system, thus producing a significant anti-cancer response. FIG. 8 demonstrates that anti-CEACAM1 antibodies and anti-PD-1/PD-L1/PD-L2 antibodies are able to bind their respective targets on tumor cells and/or immune cells in-vivo, and that this combined binding significantly attenuates tumor progression compared to each mono-therapy. This result is highly important, as it attests to the efficacy and potential of the use of a combined anti-CEACAM1 and anti-PD-1/PD-L1/PD-L2 even in established tumors of considerable volumes, which mimic the clinical setting where patients with established tumors are being treated.

### Example 4. Combination Treatment with Humanized mAb to CEACAM1 and LAK Cells

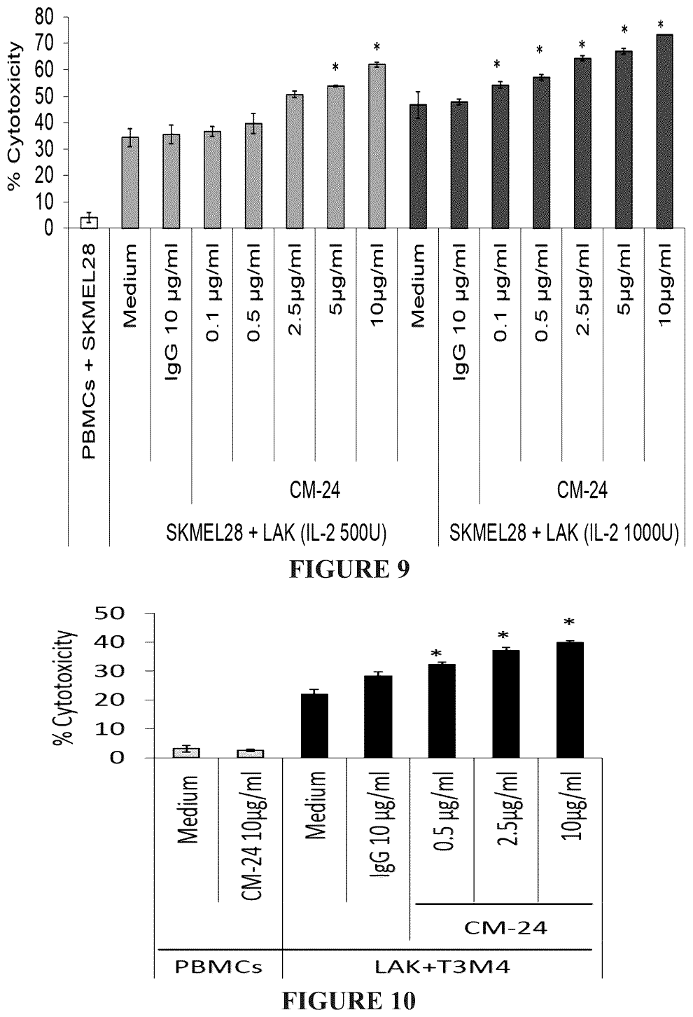

A. Anti-CEACAM1 antibodies increase the cytotoxicity of human LAK cells against human melanoma cells: PBMC cells were isolated from a healthy donor followed by activation with IL-2 (500 or 1000 units/ml) for 3 days to generate a population of human LAK cells. Then, the human LAK cells were incubated with 0.1 μg/ml, 0.5 μg/ml, 2.5 μg/ml, 5 μg/ml or 10 μg/ml of an anti-CEACAM1 antibody (CM-24) for 30 minutes at 37° C. Human melanoma cells (SKMEL28) were added for an incubation of 24 hours, after which cytotoxicity was determined. FIG. 9 demonstrates that while human LAK cells are cytotoxic to human melanoma cancer cells on their own (compare e.g. two left bars), the addition of anti-CEACAM1 antibodies significantly increases cytotoxicity to these human melanoma cancer cells in a dose-dependent manner.

B. Anti-CEACAM1 antibodies increase the cytotoxicity of human LAK cells against a variety of human pancreatic and lung cancer cells. PBMC cells were isolated from a healthy donor followed by activation with IL-2 (500 units/ml) for 7 days to generate a population of human LAK cells. Then, the human LAK cells were incubated with 0.1 μg/ml to 10 μg/ml of an anti-CEACAM1 antibody (CM-24) as indicated, for 30 minutes at 37° C. Three different human pancreatic cancer cells, T3M4, SU8686 and PANC2, and two different human lung cancer cells, H358 and H460, were added for an incubation of 24 hours. FIG. 10 demonstrates that while human LAK cells are cytotoxic to human pancreatic T3M4 cancer cells on their own, the addition of anti-CEACAM1 antibodies significantly increases cytotoxicity to these human cancer cells in a dose-dependent manner. Similar results were obtained for the other pancreatic and lung cancer cell.

C. Anti-CEACAM1 antibodies enhance granzyme B secretion of human LAK cells in the presence of human pancreatic and lung cancer cells. Human LAK cells were incubated with an anti-CEACAM-1 antibody (CM-24) in different concentrations for 30 minutes at 37° C. Human pancreatic cancer cells T3M4 (A) or human lung cancer cells H358 (B) were then added for an incubation of 24 hours. Granzyme B secretion was measured by ELISA. The results demonstrate that while human LAK cells produce high levels of Granzyme B on their own, the addition of anti-CEACAM1 antibodies significantly increases Granzyme B levels in a dose-dependent manner.

D. Anti-CEACAM1 antibodies enhance enhances IFN-γ secretion of human LAK cells in the presence of human cancer cells. Human LAK cells were incubated with an anti-CEACAM-1 antibody (CM-24) in different concentrations for 30 minutes at 37° C. Human lung cancer cells H358 or H460 were then added for an incubation of 24 hours. IFN-γ secretion was measured by ELISA. FIG. 11 demonstrates that while human LAK cells produce high levels of IFN-γ on their own, the addition of anti-CEACAM1 antibodies significantly increases IFN-γ levels in a dose-dependent manner.

### Example 5. CEACAM1 Expression Correlates with the Presence of B-Raf Mutations in Cancer Cells

A. Evaluating of biopsy samples from 24 Melanoma cancer patients for CEACAM1 expression levels and for BRAF genotype revealed that there is a statistically significant correlation between B-Raf V600E mutation and expression of CEACAM1. More specifically, whereas only 50% (3/6) of the melanoma cells having a wild type B-Raf, i.e a valine in position 600, expressed detectable levels of CEACAM1 (Ct of 36 and less), 100% (18/18) of the melanoma cells having a mutated B-Raf, i.e a glutamic acid in position 600, expressed detectable levels of CEACAM1 (Ct of 36 and less).

B. CEACAM1 extracellular staining of cancer cells treated with B-Raf or MEK inhibitors. 1.0*10⁶ cells of a B-Raf W.T. cell sample (076mel) and two B-Raf V600E cell samples (526mel, 624mel) were incubated with different concentrations of vemurafenib or selumetinib (0.1 μM or 1 μM) for 2 to 48 hours. At each time point, CEACAM1 expression on the cells was determined by FACS. Volume equivalents of DMSO (vehicle) were used as control. The results demonstrated while 0.1 μM and 1 μM vemurafenib did not have any effect on CEACAM1 expression levels on cells having W.T. B-Raf (076mel), 0.1 μM and 1 μM vemurafenib had a dose-dependent effect on CEACAM1 expression levels on cells having mutated B-Raf (526mel, 624mel). The results further demonstrate that selumetinib had a similar effect to vemurafenib on cells having mutated B-Raf (526mel, 624mel), and that while 1 μM selumetinib significantly decreased CEACAM1 expression levels on cells having W.T. B-Raf but mutated N-Ras (sk-mel-2), 1 μM vemurafenib had no effect on CEACAM1 expression levels. These results further support the regulation of CEACAM1 via the constitutively activated MAPK pathway, which is driven in this case by mutated N-Ras, and not by mutated B-Raf.

C. Inhibitor-resistant cancer cells show increase in CEACAM1 expression and restored activity of MAPK pathway. Two vemurafenib-sensitive B-Raf V600E cell samples (526mel, 624mel) and vemurafenib-resistant cell lines derived therefrom were incubated for 2 days with 1 M vemurafenib. Cells were then analyzed for CEACAM1 protein expression by FACS as described above. Vemurafenib-resistant cell lines were generated by gradual increase of the inhibitor's concentration in culture, up to 0.32 μM. It was demonstrated that vemurafenib-resistant cell lines expressed higher levels of CEACAM1 than vemurafenib-sensitive cell lines. MAPK activity was measured in vemurafenib-sensitive and vemurafenib-resistant B-Raf V600E (624mel) cell samples by immunoblotting for phosphorylated ERK1/2 (pERK, Thr202/Tyr204), total ERK1/2 and actin after 24 hours of exposure to 160 nM vemurafenib. In vemurafenib-sensitive B-Raf V600E cells vemurafenib almost completely abolishes the phosphorylation of ERK1/2, wherein MAPK activity was practically uninterrupted by vemurafenib in vemurafenib-resistant B-Raf V600E cells.

D. Inhibitor-resistant cancer cells upregulate CEACAM1 expression. B-Raf V600E 526mel melanoma cells were cultured in the presence of 1 μM vemurafenib. Cultivation was performed in RPMI 1640 supplemented with 1 mM Na-Pyruvate, 1 mM Pen-Strep, 1 mM L-Glutamine, 1 mM non-essential amino acids, and 10% heat inactivated fetal calf serum. Initial vemurafenib concentration was 0.01 of the determined IC₅₀ (0.64 nM). Each week, the concentration was doubled up to 5 times the IC₅₀ (320 nM), to generate vemurafenib-resistant melanoma cells. Cells were then tested for CEACAM1 expression using MRGI (murine antibody to human CEACAM1) in flow cytometry as described above. Total RNA was extracted with TRIZOL and cDNA was generated with a reverse transcriptase, according to routine protocols. While vemurafenib down regulates CEACAM1 expression in B-Raf V600E melanoma cells, these cells upregulate CEACAM1 expression levels upon acquiring resistance to vemurafenib. It is important to note that CEACAM1 levels in B-Raf V600E melanoma cells after acquiring resistance to vemurafenib are higher than CEACAM1 levels in untreated (vemurafenib-naïve) B-Raf V600E melanoma cells.

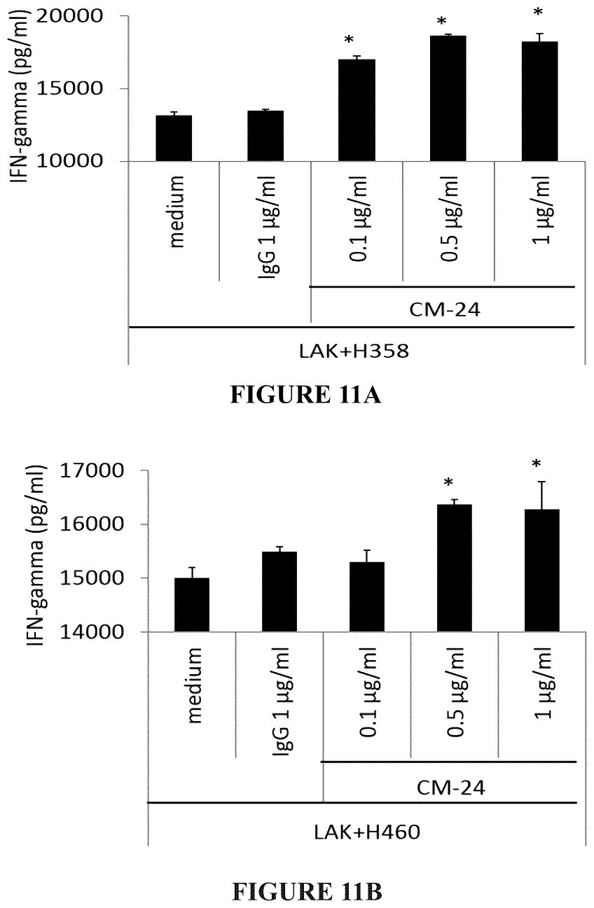

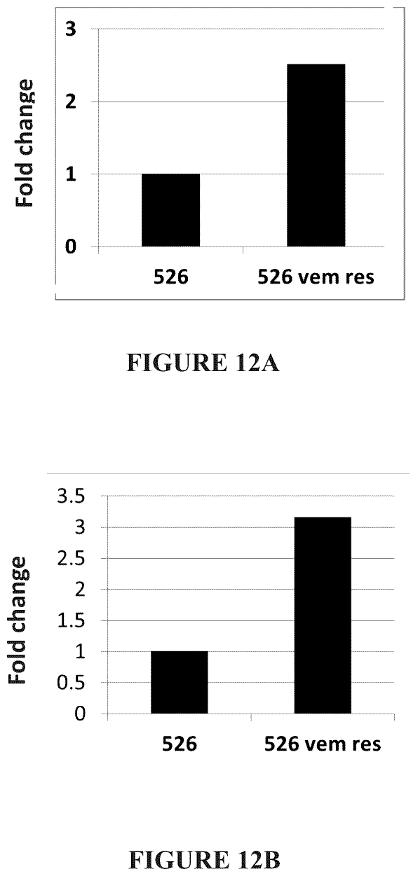

E. Inhibitor-resistant cancer cells upregulate expression of both types of CEACAM1. The vemurafenib-sensitive and vemurafenib-resistant B-Raf V600E 526mel melanoma cells mentioned above were tested for the type of CEACAM1 over-expressed upon acquiring resistance to vemurafenib by qPCR. The data presented in FIGS. 12A and 12B demonstrates that the expression of both types of CEACAM1, CEACAM1-long (12A) and CEACAM1-short (12B) is about three-fold upregulated in vemurafenib-resistant cells compared to vemurafenib-sensitive cells.

F. B-Raf/MEK inhibitors increase T-cell induced cytotoxicity. Two vemurafenib-sensitive B-Raf V600E cell samples (526mel, 624mel) were tested for viability in the presence of cytotoxic T cells, with or without 1 μM vemurafenib. Melanoma cells were pre-incubated with 1 μM vemurafenib and then co-incubated overnight with HLA-A2 matched antigen-matched T cells in effector-target ratio of 5:1. Cell killing was determined by LDH release. It was demonstrated that vemurafenib significantly sensitizes melanoma cells to cytotoxic T cells. B-Raf/MEK inhibitors and antibodies to CEACAM1 increase T-cell induced cytotoxicity to cancer cells in-vitro.

### Example 6. The Anti-Cancer Effect of CM-24 at Different Doses In-Vivo

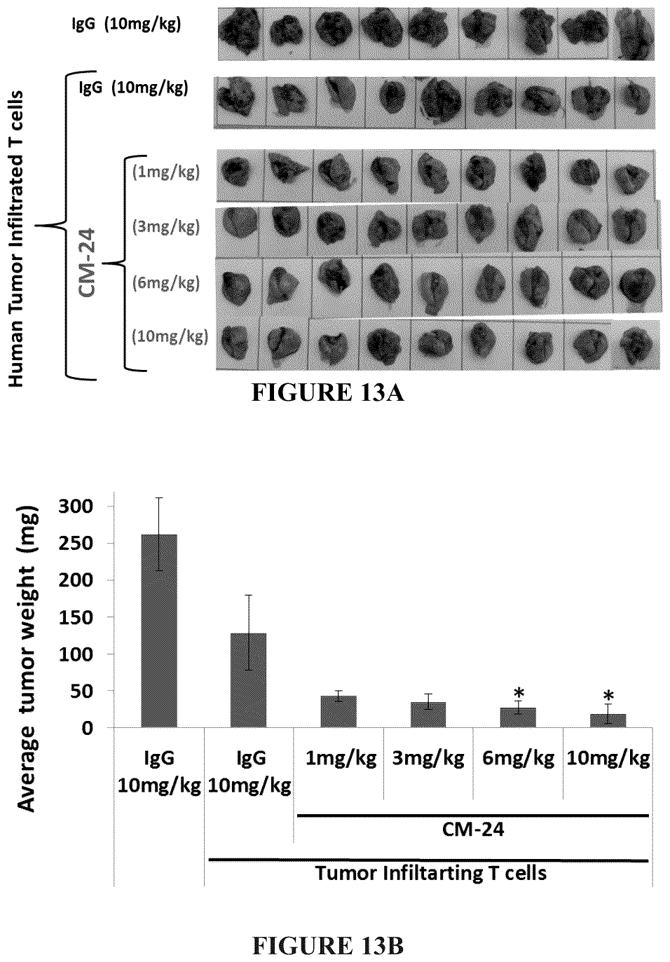

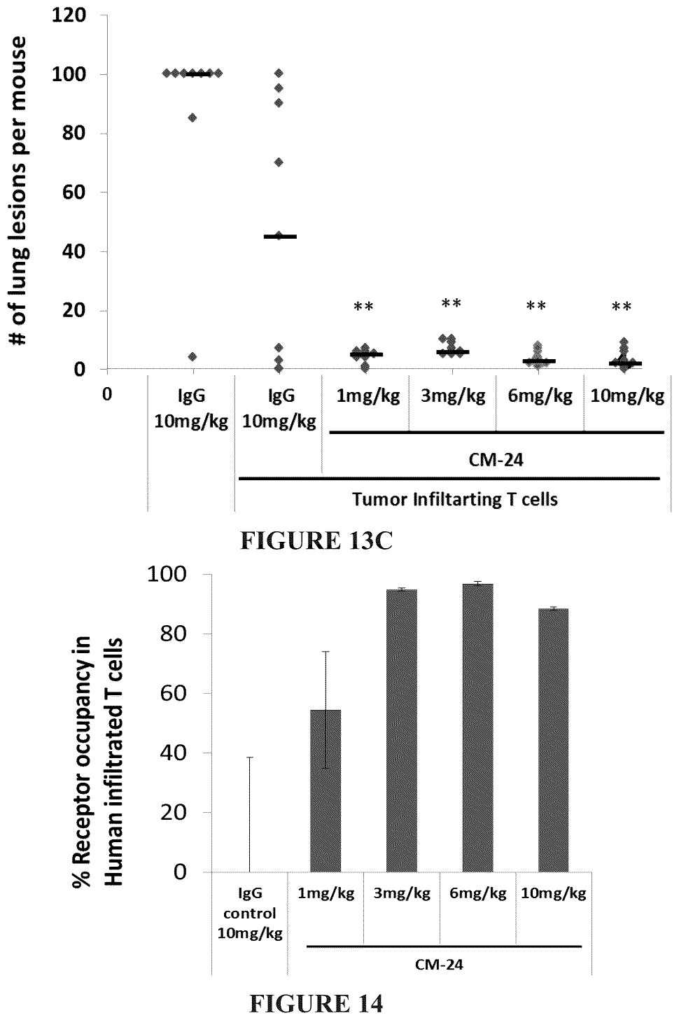

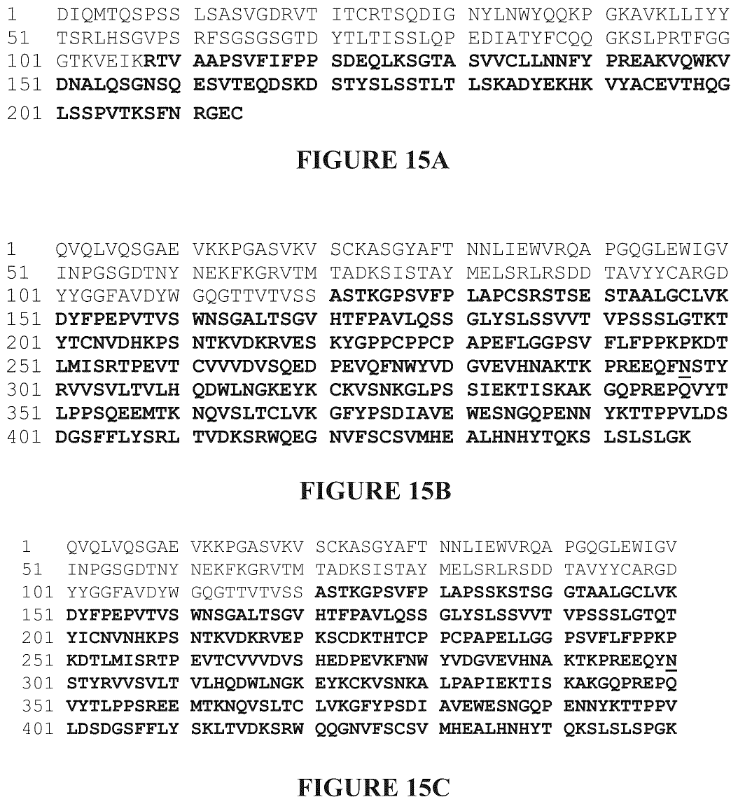

SCID-NOD mice were engrafted IV with 5×10⁶ melanoma cells (cell line MEL526) and were treated for 44 days according to the treatment groups indicated in FIG. 13. Antibodies were given twice a week by IV injection and 10×10⁶ TIL were administered IV every 10 days. At day 49 the mice were sacrificed and the lungs were removed, photographed (FIG. 13A), weighed (FIG. 13B) and the lesions were counted (FIG. 13C). FIG. 13A-Digital photos of the mice lungs immediately after harvest. FIG. 13B-Tumor weight was calculated by subtracting the lung weight of the naïve mice from the average lung weight of the different treatment groups. The results represent the average of tumor weight ±SE from 7-8 mice per treatment group. FIG. 13C - The number of lung lesions in individual mice in the various groups; the black lines represent group medians; lungs with uncountable lesions were scored as 100. Paired T test was used to calculate statistical significance between the groups * P<0.05, ** P<0.025.

Treatment with CM-24 in the presence of TIL resulted in robust tumor growth inhibition as can be appreciated by lung morphology (FIG. 13A), tumors' weight (FIG. 13B) and the number of the lung lesions (FIG. 13C), while mice treated with an IgG control showed massive tumor burden and numerous lung melanoma lesions. Only moderate tumor growth inhibition (TGI 47%) was observed in the group administrated with human reactive T cells against melanoma with a control antibody (TIL+IgG), but when CM-24 was added a substantial and dose dependent anti-cancer activity in all doses examined was demonstrated (TGI of 84%, 87%, 90% and 93% in doses of 1,3, 6 and 10 mg/kg respectively) with statistical significance in the 6 and 10 mg/kg doses (FIG. 13B) . Digital photo recordings of the lungs at the assay termination day (FIG. 13A) showed nearly normal lung morphology in mice treated with CM-24 in the presence of TIL, thus indicating that CM-24 in the presence of TIL eliminates almost completely the malignant cells.

In the group administered with TIL and the IgG control some anti-tumor effect was also observed, as was expected. However, when comparing the effect to the CM-24 treated mice in terms of TGI, number of lung lesions and lung morphology, considerable differences were observed.

Evaluation of the number of lung lesions revealed very low numbers of lung lesions in all mice treated with TIL and the various doses of CM-24 and none of these lungs showed more than 10 lesions. On the other hand, in the IgG or TIL +IgG groups several animals showed high number of lesions (>100) and the groups medians were considerably higher than the CM-24 treated mice (FIG. 13C) (100 and 45 in the IgG and TIL +IgG groups compared to 5, 6, 3 and 2, in the CM-24 treated groups; P<0.025).

These effects were observed at concentrations as low as 1 mg/ml but were statistically significant in terms of lung weight at CM-24 concentrations of 6 and 10 mg/kg.

Special notes: 1 mouse from the IgG group showed severe morbidity including limb paralysis and was sacrificed on day 33 (massive tumor burden in the lung was detected); 1 mouse from the TIL+IgG group showed morbidity signs and died on day 49, assay termination day (several lesions were detected); 1 mouse from the TIL+CM-24 group showed severe morbidity and reduced body weight and was sacrificed on day 39 (no evidence of lesions was detected); 3 mice from PBS group showed severe morbidity during the experiment and were sacrificed on day 42 and the other two mice on day 48 (massive tumor burden including high number of lesions in the lung were detected in all mice).

At termination day, a flow cytometry based assay in combination with a QuantiBrite KIT was used in order to determine CEACAM1 receptor occupancy by CM-24 in human TIL isolated from the lungs of the treated mice. Receptor occupancy (RO) values in mice treated with the various CM-24 doses demonstrated (FIG. 14) that RO of 50% could be attributed to concentrations of 1 mg/kg (CM-24 serum level in the blood) and >90% RO was demonstrated in doses of 3, 6 and 10 mg/kg (CM-24 serum levels 0.3, 48.5 and 111 μg/ml, respectively). Examination of CEACAM1 RO values was performed using a flow cytometry based assay using PE conjugated CM-24 antibody and a QuantiBrite Kit (BD) Results represent average ±SE of RO values on TIL isolated from the lung of 8-9 mice per treatment group. Data was analyzed using Kaluza software.

From the data above it is clear that substantial tumor growth inhibition was observed in mice treated with CM-24 in the presence of TIL, leading to almost complete elimination of the malignant cells. Although all CM-24 doses demonstrated effective anti-cancer responses, the increasing doses showed correlation to higher values of tumor growth inhibition (TGI of 84, 87, 90 and 93 corresponding to doses of 1,3,6 and 10 mg/kg respectively), which were also statistically significant. Ex vivo RO results demonstrate an RO >90% in doses of 3, 6 and 10 mg/kg (CM-24 serum levels 0.3, 48.5 and 111 μg/ml respectively) while RO of 50% was detected in the 1 mg/kg dose. When evaluating the RO data from mice treated with CM-24 at a dose of 1 mg/kg, although CM-24 serum concentrations are very low, the RO data demonstrates that CM-24 is still bound to the TIL in the lungs, implying that CM-24 can mediate a long lasting effect in vivo in the tumor microenvironment.

These results support the mode of action of CM-24 as an enhancer of the cytotoxic activity of the effector cells against malignant cells and show that CM-24 is a potent anti-cancer agent.

### Example 7. Summary of Pre-Clinical Data

CM-24 is a humanized IgG4 anti human CEACAM1 monoclonal antibody that binds the N terminal domain of CEACAM1 and blocks intercellular CEACAM1 interaction between activated lymphocytes and tumor cells; blockade of CEACAM1 interactions by CM-24 is therefore proposed to enhance the killing activity of lymphocytes and is a promising avenue to pursue for immunotherapy of cancer.

CM-24 shows high affinity and selective binding to human CEACAM1, which is expressed by activated lymphocytes or tumor cells. Data in in vitro immuno-modulatory models demonstrated that CM-24 is a potent blocker of intercellular CEACAM1-CEACAM1 interactions and can enhance the cytotoxic killing of various human CEACAM1-positive tumor cells by CEACAM1-positive NK and lymphokine-activated killer (LAK) cells and tumor infiltrating lymphocytes (TIL). The enhanced killing activity induced by CM-24 may be mediated by granzyme B and IFNy secretion as demonstrated in various models.

CM-24 enhances the cytotoxic activity of effector cells in the presence of CEACAM1 positive tumor cells and in the context of a specific human leukocyte antigen (HLA)-restricted T cell reaction. CM-24 does not enhance the cytotoxic activity of effector lymphocytes against CEACAM1 positive non-target cells (human normal cells). In addition, the data shows that CM-24 does not have ligand-like agonistic effects, Fc-related effector functions or direct effects. Binding of CM-24 to CEACAM1 does not induce agonistic activity, and no effect of CM-24 on effector cells could be observed in the absence of target cells as demonstrated in in vitro functional assays. CM-24 is also not expected to induce antibody-dependent cell-mediated cytotoxicity (ADCC) or complement-dependent cytotoxicity (CDC) because it is an IgG4 is unable to bind the complement component—Clq and the ADCC mediator -Fcγ-RIIIa and has no direct effect on the proliferation rate or viability of CEACAM1-positive primary cells (lymphocytes, epithelial and endothelial cells).

Various in vivo tumor xenograft models demonstrate that CM-24 has clear anti-cancer activity, which is accompanied by an increase in immunological activity of T cells within the tumor.

CEACAM1 blockade would alleviate CEACAM1-mediated inhibition of CEACAM1-positive tumor-infiltrating lymphocytes encountering CEACAM1-positive cancer cells within the tumor microenvironment. The immunological effect is expected to be enhancement of the local immune response against tumor cells, which is expected to result in their elimination and subsequent clinical regression.

Pre-clinical evaluation of the safety of CM-24 included assessment of the effect of CM-24 on T cell proliferation and pro-inflammatory cytokine secretion. Human peripheral blood mononuclear cells (PBMCs) and whole blood from 10 healthy donors were evaluated and the antibody was presented by both soluble and immobilized form. There was no apparent CM-24-induced proliferation and no significant pro-inflammatory cytokine secretion in soluble or dry-coated immobilized stimulation formats.

A 6 week repeat dose study was performed in rhesus monkeys to evaluate the toxicity and toxicokinetics of CM-24. CM-24 was administered via intravenous (IV) infusion (the intended clinical route of administration) once every two weeks for a total of 4 doses (mimicking the intended treatment cycle in oncology patients) at doses representing 2.5× (25 mg/kg) and 10× (100 mg/kg) the projected maximal dose in humans (10 mg/kg). No obvious treatment related adverse reactions, no gross or microscopic pathological findings, and no mortality were observed. Ophthalmoscopy and electrocardiogramdicated no findings related to the treatment. In addition, all blood and urine tests were found to be within normal ranges for Rhesus monkeys. Taken together, the pivotal GLP repeat-dose toxicology study in Rhesus monkeys showed no treatment-related toxicities at any dose level, and the No Observed Effect Level (NOEL) was determined to be 100 mg/kg. The toxicokinetics evaluation showed a dose-proportional increase in CM-24 exposure (Cmax, AUC) when administered by IV infusion. A slight increase in Cmax and AUC on Day 42 as compared to Day 0 might suggest the potential for some CM-24 accumulation when the mAb is administered by this ROA and dosing regimen at these dose levels. Only a single animal in Group 2 (25 mg/kg) showed a positive anti-CM-24 antibody response, suggesting that ADA response most likely had no effect on either pharmacokinetics or toxicity study. This result does not represent a strong immunogenicity response to repeated dosing with CM-24 results.

The safe clinical starting dose for CM-24 based on the MABEL determined from the in vivo experiments in the mouse tumor xenograft models results is in the range of 0.2-1 mg/kg, comprising a 10 fold safety factor. The pivotal GLP repeat-dose toxicology study in Rhesus monkeys showed no treatment-related toxicities at any dose level, and the No Observed Effect Level (NOEL) was determined to be 100 mg/kg (human equivalent dose (HED) of 100 mg/kg on a weight-to-weight basis), which clearly supports the range determined by the MABEL evaluation. The results of the in vitro/ex vivo PBMC proliferation and pro-inflammatory cytokine production assay demonstrated no substantial CM-24 related induction of T cell proliferation and pro-inflammatory cytokine production.

### Example 8. A Phase 1, Open-Label, Multicenter, Multi-Dose Escalation Study of CM-24 in Subjects with Selected Advanced or Recurrent Malignancies

Six tumor types—melanoma, non-small cell lung cancer adenocarcinoma, gastric, colorectal, bladder and ovarian cancer - have been selected for the current study, as they are representative of tumors for which a high medical need for new therapies exist; those for which there is a precedent for clinical responses to other immunotherapies; and those for which there is supportive correlative pathologic data suggesting that the CEACAM1 pathway is important for tumor progression.

The study includes 2 phases: The Dose Escalation Portion and the Expansion Cohort Portion. The Dose Escalation Portion last at least 12 weeks beginning with 4 infusions (Cycle 1) for each subject. Subjects with no Dose Limiting Toxicity (DLT) and who show evidence of clinical benefit (Complete response—CR, Partial Response—PR, Stable Disease—SD) as well as subjects with Progressive Disease-PRD on imaging assessment who are otherwise clinically stable are treated for up to a total of 5 additional cycles (Continued Treatment Period) that last up to an additional 38 treatment weeks and at least 25 weeks of follow up (total 75 weeks/approx. 15 months). The Expansion Portion last up to 46 treatment weeks and at least 25 weeks of follow up (total 71 weeks/approx. 14 months) in cutaneous melanoma subjects.

### Primary Objectives:

1. Assess the safety and tolerability of escalating multiple doses of CM-24 (administered intravenously) and

2. Determine the recommended Phase 2 dose of CM-24, in subjects with advanced or recurrent malignancies including melanoma, non-small cell lung adenocarcinoma, and gastric, colorectal, bladder and ovarian cancers.

### Secondary Objectives Include:

1. Characterize the pharmacokinetic profile of multiple doses of CM-24.

2. Characterize the immunogenicity of CM-24.

3. Evaluate preliminary efficacy on the basis of objective tumor response and duration of response in subjects treated with CM-24.

### Explorative Objectives Include:

1. Explore a potential predictive biomarker associated with CM-24 clinical activity based on levels of expression of CEACAM1 in tumor specimens prior to treatment.

2. Investigate the immuno-modulatory activity of CM-24 on selected immune cell populations and soluble factors in tumors and peripheral blood.

3. Assess the overall survival in subjects treated with CM-24.

The Dose Escalation Portion: Study drug dosing is scheduled to be administered in a staggered manner starting with 0.01 mg/kg, and continuing to 0.03 mg/kg, 0.1 mg/kg, 0.3 mg/kg, 1 mg/kg, 3 mg/kg, and 10 mg/kg. Subjects will be assigned to a dose level in the order of study entry. For the first two dose levels (0.01 mg/kg and 0.03 mg/kg) 1 patient in each cohort is enrolled in an accelerated design in which a single grade 2 drug related toxicity results in expansion to a 3+3 design at the dose and all subsequent doses. For subjects in the lower two cohorts (0.01 mg/kg and 0.03 mg/kg) Dose escalation from the first single patient low dose cohort (0.01 mg/kg) to the next cohort (0.03 mg/kg), and from the second single dose cohort (0.03 mg/kg) to the next cohort (0.1 mg/kg) commence after a 6 week DLT window, if no Grade 2 or greater toxicity has occurred. For dose levels of 0.1 mg/kg and above, at least 3 patients per cohort are enrolled in a standard 3+3 design unless a DLT occurs, in which case the cohort is expanded to 6 patients. Escalation to the next cohort only commence after an 8 week DLT window, beginning from the first study drug administration of the first subject of each cohort.

The Dosing Period includes three periods: Screening, Dosing and Follow-up: 1) 4 repeat doses that comprise the first Cycle followed by a 6 week observation period), 2) a Continued Treatment Period (cycles 2-6) and 3), a Follow-Up Period including assessment of overall survival. Each treatment Cycle comprises of 4 doses of study drug administration every 2 weeks. At least 3 subjects per cohort are enrolled in a standard 3+3 design. The minimum planned number of subjects enrolled in this portion is 17 but can increase if Dose Limiting Toxicities (DLTs) occur. If a DLT occurs at a given dose level, this dose level is expanded to 6 subjects, thus the maximum number of subjects in the Dose Escalation Portion is 42.

Enrolled subjects receive 4 treatments, once every 2 weeks (Cycle 1) followed by a 6 week Observation Window; when appropriate subjects enter Continued Treatment period during which they receive additional Cycles (2-6). During this Continued Treatment Period, subjects undergo clinical and laboratory assessments including physical examination (body weight, vital signs and oxygen saturation), pharmacokinetics and pharmacodynamics, cytokine collection as well as safety laboratory testing and immune safety assays and ECG is being recorded. All subjects undergo response evaluation one week and five weeks following Cycle 1, based on imaging and clinical assessments. Once eligible to continue in the study, additional response evaluations is performed immediately before the beginning of the next cycle. After the last continued treatment cycle, subjects are followed for safety, efficacy and survival.

The Expansion Portion: Up to 20 metastatic cutaneous melanoma subjects are enrolled and treated at the recommended Phase 2 dose (RP2D). It includes 3 periods: Screening, Dosing and Follow-Up. The Dosing Period consists of 6 cycles of 4 treatments each administered every 2 weeks. The Follow-Up Period includes assessing overall survival.

Enrolled subjects receive the recommended Phase 2 dose every 2 weeks in each cycle. Four treatments are administered and the subjects continue treatment up to 6 cycles. During the Dosing Period, subjects undergo clinical and laboratory assessments as detailed above.

Subjects must have a “wash out” period of at least 4 weeks prior to first study drug administration from all previous chemotherapy and experimental agents except for immuno-modulators (for example, but not limited to: anti-CTLA4, anti-PD-L1, anti-PD-1 antibodies, IL-2) which must have a “wash out” period of at least 6 weeks prior to first study drug administration, and all adverse events have either returned to baseline or stabilized at Grade 1 or less.

Melanoma subjects with B-Raf V600E or V600K mutation-positive melanoma must have progressed on, or were intolerant to, prior B-Raf- and/or MEK-inhibitor therapy.

### Dose Escalation Portion

Cycle 1 will consist of a 6-week Repeat Dosing Period (4 doses, each 2 weeks apart); and a 6-week observation period.

A minimum of 1 week must elapse between the first treatment of any subject in a dose cohort and the first treatment for the subsequent subject in that dose cohort.

Subjects are followed closely during the 12-week Initial Study Period. All subjects undergo response evaluation one week and five weeks after the end of Cycle 1 for evidence and confirmation of clinical benefit, defined as stable disease (SD), partial response (PR) or complete response (CR) by Week 12. Subjects with evidence of Progressive Disease (PRD) on either Week 7 or 11 imaging after being reconsented on the Informed Consent Form (ICF), can continue study participation and continued CM-24 treatment, if investigator deems it is clinically warranted according to Stopping Rules of Clinical Deterioration Section and further evaluated at week 15. If follow-up imaging at Week 15 confirms PRD, the subject does not continue treatment due to confirmed disease progression.

All subjects with DLTs, including delayed DLTs, will discontinue further dosing with CM-24 but are not withdrawn from the study.

Subjects who are withdrawn from the study before completion of the first 3 study drug administrations in the Initial Study Period (Cycle 1) are replaced. Subjects who are withdrawn for any other reason other than withdrawal of consent are followed over 4 follow-up visits for a period of six months.

Cycles 2-6 are referred to as the Continued Treatment Period. Following the Initial Study Period, subjects with evidence of clinical benefit, defined as SD, PR or CR according to modified RECIST 1.1 criteria and no evidence of Dose Limiting Toxicity (DLT) by Week 12, may continue treatment with CM-24 at the same dose level received during the Initial Study Period for 5 additional treatment cycles. Subjects with PRD that has been confirmed but is not worsening and with otherwise stable or improved clinical status should continue to be treated with study drug until there is further progression or clinical deterioration.

Each full treatment cycle of the Continued Treatment Period comprises 4 doses of study drug administered 2 weeks apart, on Days 1, 15, 29, and 43. A response assessment is performed between Days 52 and 56 of each treatment cycle. The response assessment must be completed before the first dose of the next cycle is administered.

During the Continued Treatment Period, subjects with PRD that has been confirmed but is not worsening and with otherwise stable or improved clinical status should continue to be treated with study drug until there is further progression or clinical deterioration.

After the last administration of CM-24 in the Continued Treatment Period, each subject is followed over 4 follow-up visits for a period of six months.

All subjects with DLTs, including delayed DLTs, discontinue further dosing with CM-24 but are not withdrawn from the study.

All subjects are followed indefinitely for survival.

### Cohort Expansion

For the first two dose levels (0.01 mg/kg and 0.03 mg/kg) 1 patient in each cohort is enrolled in an accelerated design in which a single grade 2 drug related toxicity results in expansion to a 3+3 design at the dose and all subsequent doses. For subjects in the lower two cohorts (0.01 mg/kg and 0.03 mg/kg) Dose escalation from the first single patient low dose cohort (0.01 mg/kg) to the next cohort (0.03 mg/kg), and from the second single dose cohort (0.03 mg/kg) to the next cohort (0.1 mg/kg) are commence after a 6 week DLT window, if no

Grade 2 or greater toxicity has occurred. For dose levels of 0.1 mg/kg and above, at least 3 patients per cohort will be enrolled in a standard 3+3 design unless a DLT occurs, in which case the cohort is expanded to 6 patients. Escalation to the next cohort will only commence after an 8 week DLT window, beginning from the first study drug administration of the first subject of each cohort.

If no additional subject in the 6-subject cohort has a DLT, then following review of safety data for all subjects by the Safety Committee, dose escalation may proceed.

If 2 or more subjects in a 3- or 6-subject dose cohort develop DLTs, dose escalation is stopped, and:
If the preceding dose level cohort was not already been expanded to 6 subjects, it will be expanded to 6 subjects.
If the previous dose level cohort was already expanded to 6 subjects, the Safety Committee, after review of all safety data to date, may:
   Deem the (previous) dose level to be the recommend Phase 2 dose (RP2D), or Recommend evaluation of a new cohort at an intermediate dose.
   The recommended Phase 2 dose (RP2D) is defined as the highest dose level at which no more than 1 out of 6 subjects experiences a DLT.

### Dose Escalation

If no DLTs are encountered in the 6 week DLT window for lower two cohorts, and for remaining cohorts, an 8 week DLT window, then the next cohort is started and the same pattern repeated. There is a one-week waiting period between subject enrollments within each cohort dose level. Prior to dose escalation the available safety data for all subjects treated in the study to date, including the current cohort, are evaluated by the Safety Committee.

The dosing (cycle 1) of the next dose cohort is initiated only after the repeat dosing (cycle 1) for all subjects in the preceding dose cohort has been completed. The timing of the Safety Committee review is based upon data suggesting that irAEs in patients treated with other immuno-modulators occur within 5-10 weeks following administration. Should a DLT occur, necessitating expansion of the treatment dose cohort to six subjects, the DLT window is expanded to encompass full repeat dosing (Cycle 1) of all six subjects.

If, after the dose is escalated, if 2 or more delayed DLT's are encountered at a lower dose, and that AE could be a possible delayed DLT related to CM-24, accrual is temporarily suspended and the Safety Committee notified within 24 hours. The Safety Committee will promptly evaluate the event and relevant information, including PK data, and makes any necessary adjustment in dose and/or the dose escalation scheme. The same steps are to be undertaken 2 or more delayed DLT's occur in a previously tested, 6-subject expanded cohort.

Delayed DLTs will be reviewed by the Safety Committee following guidelines and decisions are made on a case-by-case basis. Such actions could include applying the standard DLT escalation rules, returning to a lower or intermediate dose, or taking no action if the dose-related event being examined is not serious enough to halt dose escalation and current dosing is not considered a risk to subjects.

### Expansion Portion

The Expansion Portion of this study allows enrollment of up to 20 subjects with advanced cutaneous melanoma. Other expansion arms of up to 20 subjects may be opened, subject to protocol amendment, in the indications previously studied during the Dose Escalation Portion of the trial, if early efficacy signals warrant this. Enrolled subjects are treated at the RP2D for up to six cycles, with treatment administered on Days 1, 15, 29, and 43. A response assessment is performed between Days 52 and 56. The response assessment must be completed before the first dose administration in the next cycle.

Enrolment may be held in the Expansion Portion if the rate of DLTs is ≥33%. Subjects who are tolerating study drug are not automatically precluded from continued dosing until the Safety Committee review, and are allowed to continue dosing for as long as tolerated unless directed otherwise as a result of the safety review. After safety analysis, a decision is made whether to resume enrolment and continue dosing at the current dose or continue enrolment of further subjects at a lower dose. For DLTs, enrolment is held and/or restarted following review of the Safety Committee.

### Treatment Decision Guidelines

Tumor response is evaluated using Response Evaluation Criteria in Solid Tumors (RECIST 1.1). End of cycle tumor response assessments for all subjects occur within Days 52 to 56 of each treatment cycle (results of assessments must be reviewed and documented before the first dose of the next cycle). Following each (continued or expansion) treatment cycle, the decision to treat a subject with an additional cycle of CM-24 is based on tumor assessment, unless the subject develops a ≥Grade 3 (CTCAE) adverse event or other adverse event related to CM-24 that precludes further treatment. Subjects are treated until confirmed complete response (CR) or progressive disease (PRD) that is both confirmed and then further progresses as described below. Subjects with PRD that has been confirmed but is not worsening and with otherwise stable or improved clinical status (i.e. no reduction in ECOG performance status) should continue to be treated with study drug until there is further progression or clinical deterioration, as further elaborated below.

Subjects with a Best Overall Response (BOR) of CR, PR or SD continue to receive CM-24 treatment until the first occurrence of either:
Achievement of a confirmed CR;
Clinical deterioration suggesting that no further benefit from treatment is likely;
Meets criteria for discontinuation of study therapy (DLT) or other intolerability to therapy; or
Receipt of the maximum number of cycles.

### Follow-Up Period

Subjects are followed for a period of at least six months beginning from the last treatment infusion. The 1^{st} follow-up visit will take place within 7 days of the last imaging scan. Follow-up visits 2-4 will take place at 56 day intervals. In addition, survival status is assessed approximately every 3 months, indefinitely, by either a telephone call or in-person contact, following completion or discontinuation of the treatment, and follow-up of the study. Dates of death are reported for any subjects that are deceased. Overall survival assessments are made until study completion or termination by the Sponsor. No other data (e.g., subsequent therapies, performance status etc.) other than survival is collected during these calls or visits.

When a subject discontinues study drug treatment, the date and reason for study drug discontinuation should be documented in the source documents, and the subject should enter the Follow-up Period. When a subject withdraws from the study (during the Treatment or Follow-up Period), all efforts should be made in order to ensure that all evaluations associated with that study visit are performed and the date and reason for study discontinuation are documented in the source documents.

Following completion of the treatment and follow-up periods, all surviving subjects are followed for survival status every 3 months, indefinitely.

### Physical Description of Study Drug

CM-24 is supplied in a single-use 10mL vial. Each vial contains a concentrated solution with the equivalent of 100 mg CM-24 (10 mg/mL).

CM-24 is administered as an intravenous infusion, with a 0.2 micron in-line filter at the protocol-specified doses.

### Instructions for Preparation of the Different Doses

For subjects receiving doses of 0.1 mg/kg, 0.3 mg/kg and 1.0 mg/kg, a 50 mL 0.9% sodium chloride IV bag is used for the preparation. Infusion should proceed at a rate of 1.0 mL/minute. Rounding during dose preparation should be performed only when absolutely necessary and should only be done in a manner that will allow the minimum concentration of 0.25 mg/mL to be maintained.

For subjects receiving dose of 3 mg/kg, a 100 mL 0.9% sodium chloride IV bag is used for the preparation. Infusion should proceed at a rate of 2.0 mL/minute. Rounding during dose preparation should be performed only when absolutely necessary.

For subjects receiving dose of 10 mg/kg, a 250 mL 0.9% sodium chloride IV bag is used for the preparation. Infusion should proceed at a rate of 3.0 mL/minute. Rounding during dose preparation should be performed only when absolutely necessary.

### Pharmacodynamic (PD) Markers

Blood samples are taken and tested for immune assays and other evaluations at the following time points: pre-dose of 1^{st} study drug administration, 48 hours after the 1^{st} and 4^{th} dose of Cycle 1 study drug administration, and pre-dose of 4^{th} (last) dose of each complete cycle (cycles 2-6 of dose escalation, and all cycles of the expansion cohort), Follow-up Visits 2-4, and include:
lymphocyte subtypes (CD4, CD8 and CD56) in combination with activation markers (CD69, CD107a and HLA-DR), Regulatory T cells by fluorescence-activated cell sorting (FACS)
CEACAM1 expression in lymphocyte subtypes
soluble CEACAM1 and Granzyme B in serum
percent CEACAM1 receptor occupancy by CM-24
myeloid derived suppressor cells (MDSCs) (CD14+, HLADR low, CD11b+)
immune checkpoint proteins, for example PD-1, TIM-3, LAG, Vista

### Pharmacokinetics (PK)

Pharmacokinetics is initially studied during the Dose Escalation Portion during the first infusion (first dose of Cycle 1) and during the fourth infusions (last dose of Cycle 1). Pre-dose levels are also taken before each treatment in the 1st cycle. Additional Dose Expansion subjects (up to 6) may be tested for PK evaluation at the preliminary RP2D if more robust PK characterization of CM-24 is deemed warranted.

The PK profile of CM-24 is assessed in plasma up to 15 days post dose for the first dose and 36 days post-infusion for the fourth dose. The following PK parameters are derived from the plasma concentration versus time profiles: Cₘₐₓ, t_{1/2}, Tₘₐₓ, , AUC₀₋ₜ, and AUC_{0-∞}.

Samples are taken at the first and fourth infusions at the following time points: pre-infusion (baseline); at the end of the infusion and at 1, 4, 8, 24 hours post-infusion. For the 1st and 4th doses only: on Days 3, 5, 8, 15 (or pre-dose of next treatment) and for the 4^{th} dose also on 22 and 36 days post infusion. Pre-dose levels will be taken before each treatment in the 1st cycle. Refer to the Schedule of Events and the Laboratory Manual for further information on sample collection and shipment of samples.

### Fresh Tumor Biopsies

Tumor samples are evaluated for the following: CD4, CD8, CD56, FOXp3, CEACAM1, Granzyme B, CM-24, % CEACAM1 receptor occupancy by CM-24.

Dose Escalation Portion: Subjects are asked to provide an optional fresh biopsy tissue (or archived if taken within the past six months) sample at baseline and a week after the second treatment of the 1^{st} cycle.

Expansion Portion: Two fresh tumor samples are taken, at Screening and one week after the 2^{nd} dosing administration of the 1^{st} cycle. In addition, subjects are encouraged but not required to provide one additional biopsy a week after the fourth treatments of the 1st cycle.

### Efficacy Endpoints

The following endpoints are used to assess preliminary efficacy, and are derived from the modified RECIST 1.1 criteria:
Objective Response Rate (ORR)
Duration of Response (DOR)
Tumor Response Status
Disease Control Rate (DCR)
Durable Response (DR)
Best Overall Response (BOR) - Complete Response (CR), Partial Response (PR), Stable Disease (SD) and Progressive Disease (PRD).
Progression Free Survival (PFS)
Time to Response (TTR)
Overall Survival (OS)
Percent Change in Tumor Burden (PCTB), assessed by CT or MRI.
   The listed above efficacy endpoint will be evaluated at:

### Dose Escalation Cohorts:

Week 7, one week after the fourth dose of the first cycle was administered.
Week 11, five weeks after the fourth dose of the first cycle was administered
Week 18-19, Week 26-27, Week 34-35, Week 42-43, Week 48-49 and Week 50-51, after the five additional cycles, plus follow-up visits 2-4.

### Dose Expansion Cohort:

Week 7-8, Week 14-15, Week 22-23, Week 30-31, Week 38-39, Week 46-47, plus follow-up visits 2-4.

### Immune-Related Efficacy Endpoints

Additional exploratory efficacy evaluations may include the application of an immune-related response criteria (irRC) based on modifications to the RECIST 1.1 (referred to as irRECIST) and include the following endpoints.
Immune-related best overall response (irBOR) with response categories irCR, irPR, irSD, irPD;
Immune-related objective response rate (irORR) during the entire study period;
Duration of ir responses (DOirR) for those subjects with ir-responses;
The irORR based on the irBOR outcomes in any number of cycles may also be derived.

### Pharmacokinetic (PK) Endpoints

Pharmacokinetic is studied during the Dose Escalation Portion during the following PK time points: Samples will be taken at the first, and fourth infusions at the following time points: pre-infusion (baseline); at the end of the infusion and at 1, 4, 8, 24 hours post-infusion. For the 1st and 4th doses: on Days 3, 5, 8, 15 (pre-dose of next treatment) and for the 4^{th} dose also at, 22 and 36 days post infusion. Pre-dose levels are taken before each treatment in the 1st cycle. Additional Dose Expansion subjects (up to 6) may be tested for PK evaluation at the preliminary RP2D if more robust PK characterization of CM-24 is deemed warranted.

### Example 9. Formulation

An exemplary formulation of a humanized mAb according to the present invention comprises the following:

| Concentration (mg/ml) | Ingredient |
|---|---|
| 10.00 | CM-24 Drug Substance |
| 4.65 | L-Histidine |
| 82.00 | Sucrose |
| 0.20 | Polysorbate 20 |

### Example 10. Clinical Trial—CM-24 Plus Nivolumab in Patients with Solid Tumors

The present invention is based in part on the results of a clinical trial conducted to evaluate the safety and efficacy of the anti CEACAM1 mAb CM-24 (NCT04731467)(FW2020-01). Part A of the trial assessed the safety of CM-24 administration and involved administration of CM-24 in combination with the anti-PD1 mAb Nivolumab, in dose escalation, to patients with solid tumors. Part C2 included 8 additional pancreatic cancer patients who were administered with CM-24 in combination with Nivolumab, and the chemotherapeutic cocktail Nal-IRI/5FU/LV.

In a dose escalation arm of the trial (part A), adult subjects with selected recurrent or metastatic solid tumors, were treated with CM-24 at doses of 10 mg/kg (3 patients), 15 mg/kg (3 patients), and 20 mg/kg (5 patients) by 1-hour infusion, followed by 30-minutes infusion of Nivolumab 480 mg/patient, every two weeks. In part C2 of clinical trial NCT04731467, patients were administered with 20 mg/kg of CM-24 followed by 30-minutes infusion of Nivolumab 480 mg/patient. The dosage of Nal-IRI was 70 mg/m2 intravenously over 90 minutes. Leucovorin (LV) at 200-400 mg/m2 was administered via a slow injection of at least 3 minutes at the end of Nal-IRI infusion subsequent to which, infusion of 2400 mg/m2 5FU was initiated.

Pre-treatment tumor biopsies (core needle biopsies) were collected from all subjects in all parts of the study who have been found eligible for the study for the assessment of CEACAM1 expression on tumor cells and tumor infiltrating lymphocytes. Archival tumor biopsies were included in cases where pretreatment tumor biopsies were not available. Subjects whose treatment was ceased during cycle 1 were not included in the analysis described herein.

### CEACAM1 Immunohistochemical Staining Protocol

MRG1—a murine anti human CEACAM1 monoclonal Antibody (IgG1 kappa isotype), described in WO2010/125571 and further developed by Precision Ab, Onc., Homebrew. Cat# KTOV001-MRG1. Lot#MRG1-27Feb2020 was used for staining CEACAM1.

### Staining Overview

| | HIER temp | Antibody | | |
|---|---|---|---|---|
| HIER solution | start/run/cool | Concentration | Time | Detection |
| pH6 (ER1) | 20 min at 90° C. | 0.2 μg/ml | 60 min | 8 min |

### Tumor Microarray (TMA) Slides

Pancreatic TMA (PA1001c, US Biomax, currently known as PA1001a).

Pancreatic TMA with the patient survival information (HPanAde180Sur-0, US Biolab). All 92 cores of PDAC were scored and analyzed.

### Human CEACAM1 Staining Protocol

A 3,3′-Diaminobenzidine (DAB) staining assay was performed on the Leica Bond RX Autostainer using the murine anti human CEACAM1 mAb MRG1 (mouse IgG1 kappa from Precision Ab, Inc.). The MRG1 mAb was used at a final concentration of 0.2 μg/ml, and the Leica Bond Polymer Refine Detection Kit (BPRD, #DS9800) was used for visualization of signal. Slides were cut at 4 microns, allowed to air dry, then baked for 60 min at 60° C. Baked slides were placed on the Leica slide racks, cover tiles were placed on slides, and racks were inserted into the Autostainer. The MRGI antibody was diluted to its final concentration of 0.2 ug/ml using Leica antibody diluent (#AR9352). Once slides were on the Autostainer, Bond Dewax Solution was applied 3 times at 72° C. followed by the application of 100% Ethanol 3 times, followed by the application of Leica Bond Wash 1X solution 3 times. For antigen retrieval, Leica Epitope Retrieval Solution 1 pH6.0 (#AR9661) was incubated on the slides for 20 min at 90° C., followed by the application of Leica Bond Wash 1X solution 5 times. Thermo Pierce Protein-Free Blocking Buffer was applied for 5 min, followed by incubation with the diluted MRGI antibody for 60 min. Slides were then washed 3 times with Leica Bond Wash 1X solution. For detection, first a rabbit anti-mouse IgG Post Primary Link (from the BPRD kit) was applied for 8 min, followed by washing with Leica Bond Wash 1X solution applied 3 times for 2 min each time. Then an anti-rabbit poly-HRP IgG Polymer (from the BPRD kit) was applied for 8 min, followed by washing with Leica Bond Wash 1X solution applied twice for 2 min each. Next a DI water rinse was performed followed by a peroxide block for 10 min. Slides were then washed twice with Leica Bond Wash 1X Solution followed by a DI water rinse. DAB chromogen/substrate was applied for 10 min followed by washing with Leica Bond Wash 1X solution 3 times. Hematoxylin counterstain was applied for 2 min followed by a DI water rinse, then a Leica Bond Wash 1X Solution 1 time, and a DI water rinse. Slides were then removed from the Autostainer and cover slipped manually as follows: two changes of 95% Ethanol for 1 min each, two changes of 100% Ethanol at 1 min each, two changes of Xylene at 2 min each, and coverslip with Cytoseal mounting media.

Total TILs were determined using methods know in the art, for example fluorescent and histological methods. In some of the experiments, staining of CD3, using specific antibodies, was used to determine total TILs in the samples.

### Scoring & Evaluation of Human CEACAM1 Staining

Samples were qualitatively evaluated by the same Lead Pathologist for intensity and distribution in appropriate target cells and subcellular localization, and for the absence of non-specific background/off-target staining in negative control tissue. For positive control, TMA and patient samples, staining for CEACAM1 was evaluated in tumor cells and immune cells as detailed below, using the mAb MRG1.

For whole tissue sections, the area of interest for the estimation of CEACAM1-positive lymphocytes was determined by the Lead Pathologist according to the following criteria. All tumor compartment was part of the area of interest, including the stroma within 200-500 um from the tumor.

The CEACAM1 positivity in lymphocytes in whole tissue was expressed as a percentage (percent positive lymphocytes) according to the following formula:

The number of MRG1-positive lymphocytes divided by the total number of lymphocytes (at the same area), multiplied by 100. The total number of lymphocytes was estimated by the same Lead Pathologist based on H&E staining in FW2020-01 Study patient samples, and the TMA PA1001c samples used for method development.

For the TMA (given the smaller size of the reviewable area), individual TMA cores represented the area of interest.

The relationship between PDAC patient survival and the expression levels of CEACAM1 in tumor-associated immune cells, e.g., tumor infiltrating lymphocytes (TILs) was investigated in pre-dose biopsies of patients who were then treated with either CM-24 and Nivolumab (Part A) or CM-24, Nivolumab and Nal-IRI/5FU/LV (Part C2). Quantitative multiplex immunohistochemistry was used to evaluate CEACAM1-positive lymphocytes in pre-treated patient biopsies:

An analysis of CEACAM1-positive lymphocytes (cutoff of ≥8%) and overall lymphocytes (cutoff of ≥2%) data from biopsies taken pre-treatment from 8 evaluable PDAC patients of Part A who were treated at least three times withCM-24 and Nivolumab, was performed.

TABLE 8
Overall survival, percentage of CEACAM1-positive lymphocytes and
overall lymphocytes in pre-treatment biopsies of Part A patients.

| | | % CEACAMI⁺ | |
|---|---|---|---|
| Patient no. | % Lymphocytes | lymphocytes | Survival (Months) |
| A2 | 5 | 10 | 14.6 |
| A1 | 1 | 10 | 9.6 |
| A3 | 1 | 5 | 3.5 |
| A4 | 1 | 1 | 2 |
| A6 | <1 | 0 | 3.3 |
| A11 | 3 | 3 | 1.8 |
| A13 | 3 | 2 | 4.5 |
| A9 | 5 | 8 | 11.1 |

The analyses of patient biopsies demonstrated higher CEACAM1-positive lymphocytes (median of 10% vs 2.5%) in 3/8 PDAC patients with extended survival (9.6, 11.1 and 14.6 months). Multiplex staining of the biopsies revealed that the most abundant tumor infiltrating immune cells and CEACAM1-positive tumor infiltrating immune cells in PDAC were myeloid CD11b+ cells.

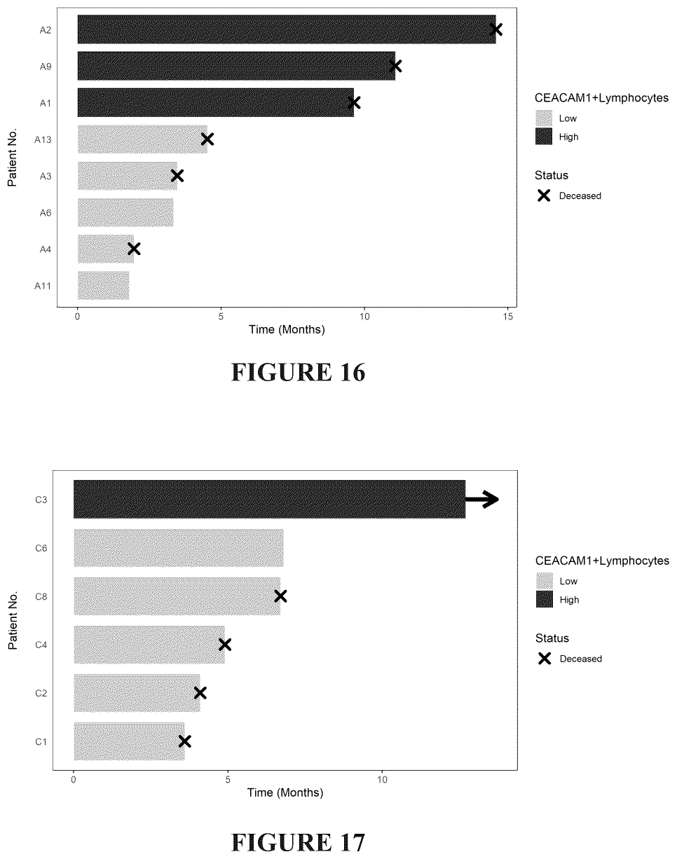

The results shown in FIG. 16 and Table 8 demonstrate an association between CEACAM1⁺ lymphocyte percentage level in PDAC patients pre-treatment and their survival on CM-24 treatment.

An analysis of CEACAM1-positive lymphocytes (cutoff of ≥8%) and overall lymphocytes (cutoff of ≥2%) data from biopsies taken pre-treatment from 6 evaluable PDAC patients of Part C2 (treated with CM-24, Nivolumab and Nal-IRI/5FU/LV) who were treated at least three times with the antibodies, was performed.

TABLE 9
Overall survival, percentage of CEACAM1-positive lymphocytes and
overall lymphocytes in pre-treatment biopsies of Part C2 patients.

| | | % CEACAMI⁺ | Survival |
|---|---|---|---|
| Patient no. | % Lymphocytes | lymphocytes | (Months) |
| C1 | 1 | 1 | 3.6 |
| C2 | 8 | 1 | 4.1 |
| C4 | <1 | 0 | 4.9 |
| C6 | 2 | 2 | 6.8 |
| C8 | 5 | 0 | 6.7 |
| C3 | 1 | 10 | >12 |

While all patients received CM-24 plus Nivolumab plus Nal-IRI/5FU/LV treatment, FIG. 17 and Table 9 show that the patient that demonstrated extended survival is the one who had high CEACAM1⁺ lymphocytes expression pre-treatment.

An analysis included 14 PDAC patients of Parts A and C2 with available pre-treatment CEACAM1-positive lymphocytes data (cutoff of ≥8%), pre-treatment overall lymphocyte data (cutoff of ≥2%), and survival data (per July 2023, one patient is still on treatment), was performed.

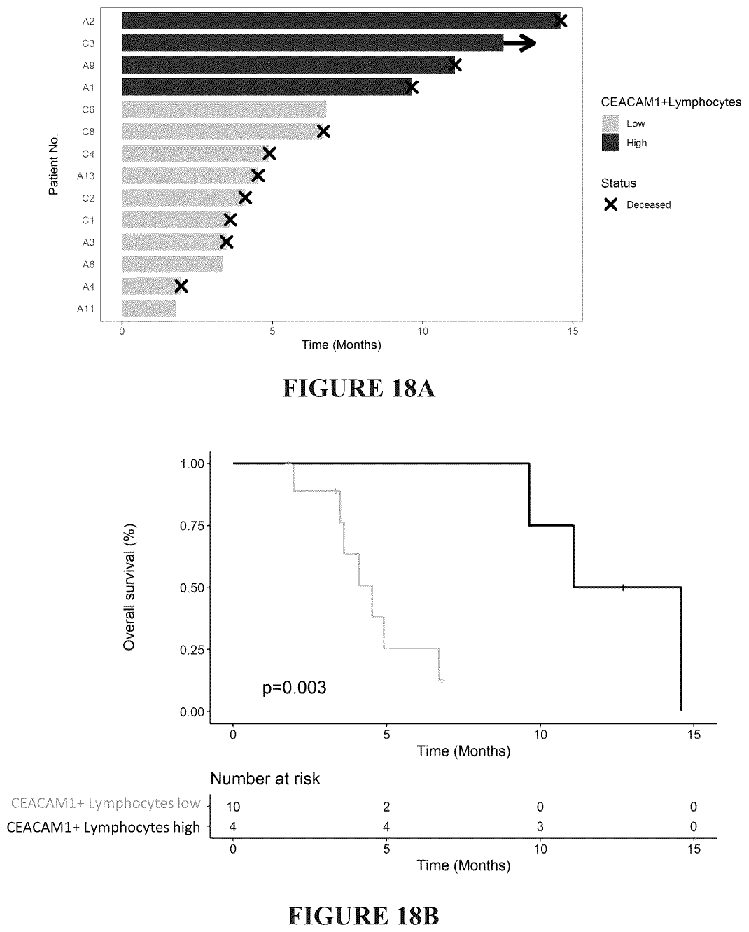

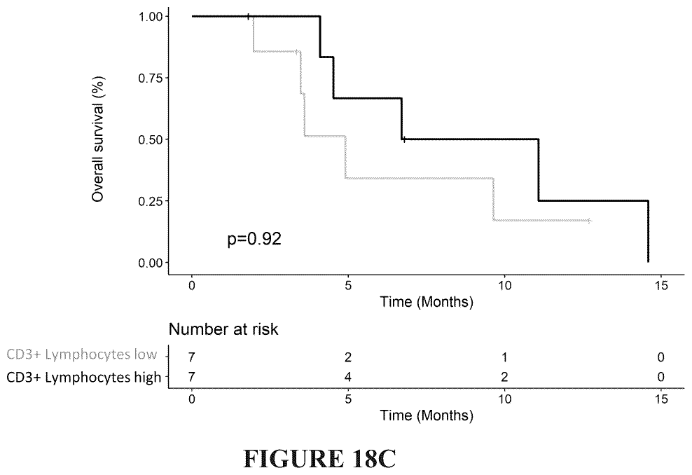

Using the available survival data, Kaplan-Meier curves have been constructed and stratified by the levels of CEACAM1⁺ lymphocytes (threshold=8) and of overall lymphocytes (threshold=2). The survival distributions were compared using the likelihood ratio test. FIG. 18A demonstrates a positive association between higher CEACAM1⁺ lymphocyte levels and overall survival of patients treated with CM-24. FIG. 18B shows that patients with high

CEACAM1⁺ lymphocyte levels were associated with significantly longer survival (median survival data from Kaplan-Meir curves: 4.5 months for low CEACAM1⁺ lymphocytes levels (0-5%) and 12.4 months for high CEACAM1⁺ lymphocytes levels (>=8%) (P=0.003). FIG. 18C shows no association between overall lymphocytes content pre-treatment and patient survival (P=0.92).

These results clearly indicate that CEACAM1⁺ lymphocytes pre-treatment levels in cancer patients are associated with the survival of these patients, when treated with CM-24; Treatment with anti CEACAM1 prolonged the survival of patients with high levels of CEACAM1⁺ lymphocytes pre-treatment.

To elucidate the distinction between the prognostic and predictive roles of CEACAM1⁺ lymphocytes, the survival trends among pancreatic cancer patients with varying CEACAM1⁺ lymphocyte expression levels were analyzed using a commercial tumor microarray (TMA) slide collection of 92 samples of PDAC patients (HPan-Ade180Sur-02, Biolabs). CEACAM1 and lymphocytes expression were assessed by immunohistochemical staining of the TMA slides. Using the available survival data, Kaplan-Meier curves have been constructed and stratified by the levels of CEACAM1⁺ lymphocytes (threshold=8) and overall lymphocytes (threshold=2). The survival distributions were compared using the likelihood ratio test.

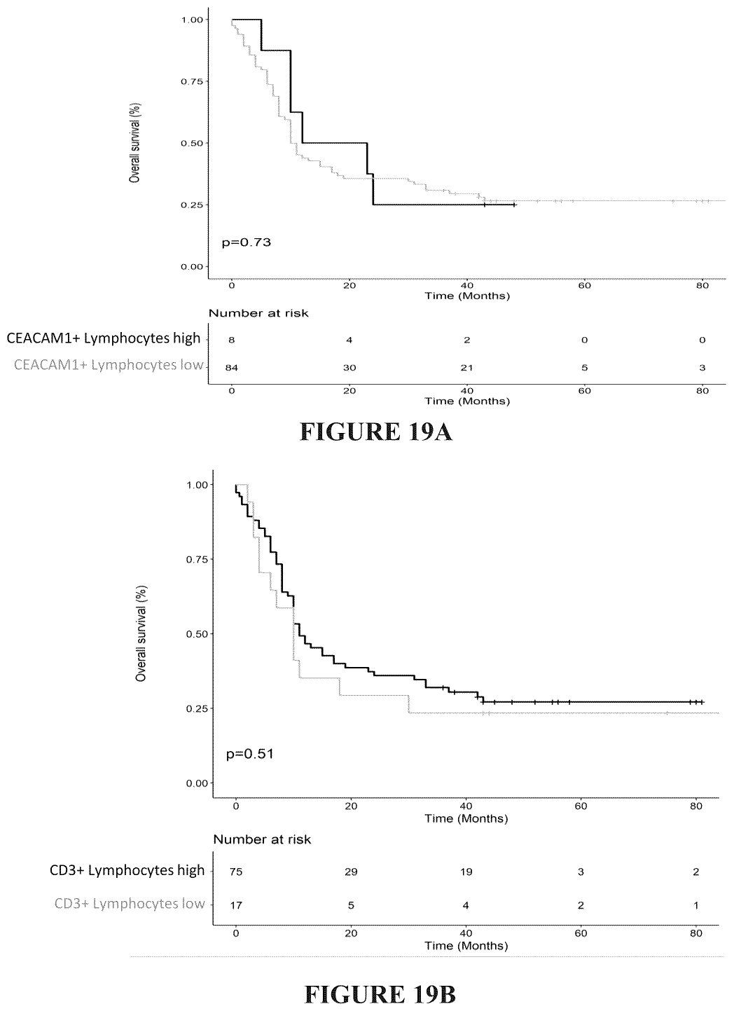

FIG. 19A shows no association between patient survival and CEACAM1⁺ lymphocyte in PDAC patients that were not treated with CM-24 (P=0.73). FIG. 19B shows no association between patient survival and overall lymphocyte in a commercial PDAC TMA (P=0.51).

Taken together, these results strengthen the previous results demonstrating that CM-24 treatment in cancer patients with high pre-treatment CEACAM1⁺ lymphocyte levels result in longer survival in these patients, whereas without anti CEACAM1 treatment, these patients' survival is comparable to patients with low pre-treatment CEACAM1⁺ lymphocyte levels. Therefore, CEACAM1⁺ tumor-infiltrating lymphocytes can be used as a biomarker to optimize patient selection and treatment.

1. A method of treating a cancer subject in need of such treatment, the method comprising:
(i) determining the level of CEACAM1 expression on tumor infiltrating immune cells (TILs) in a biological sample obtained from a subject;
(ii) comparing the level of CEACAM1 expression on the TILs from the biological sample to a reference value or to a control sample value; and
(iii) administering an anti-CEACAM1 mAb or an active fragment thereof to said subject if the level of CEACAM1 expression on TILs in said biological sample is higher than the reference value or the control sample value.

2. The method of claim 1, wherein the cancer comprises a solid tumor.

3. The method of claim 1, wherein the cancer is selected from the group consisting of: pancreatic, breast, prostate, melanoma, colorectal, bladder, lung, non-small cell lung carcinoma (NSCLC), non-small cell lung adenocarcinoma (NSCLA), gastrointestinal, thyroid, stomach, ovarian, myeloma and uterine cancer.

4. The method of claim 1, wherein the cancer is a pancreatic cancer.

5. The method of claim 1, wherein the tumor infiltrating immune cells are lymphocytes.

6. The method of claim 1, wherein the tumor infiltrating immune cells are leucocytes.

7. The method of claim 1, wherein the level of CEACAM1 expression on the TILs in the biological sample is at least 5% of the overall TILs.

8. The method of claim 1, wherein the level of CEACAM1 expression on the TILs is determined using an anti-CEACAM1 mAb or an active fragment thereof, comprising:
(i) a heavy-chain variable region amino-acid sequence comprising the three CDR sequences set forth in SEQ ID NO:1, SEQ ID NO:2 and SEQ ID NO:3; and
(ii) a light-chain variable region amino-acid sequence comprising the three CDR sequences set forth in SEQ ID NO:4, SEQ ID NO:5 and SEQ ID NO:6.

9. The method of claim 1, wherein the anti CEACAM1 mAb or an active fragment thereof administered, comprises:
i. a heavy chain variable region sequence selected from the group consisting of: SEQ ID NO:28, SEQ ID NO:29, SEQ ID NO:30, SEQ ID NO:31 and SEQ ID NO:32, and
ii. a light chain variable region sequence selected from the group consisting of: SEQ ID NO:33, SEQ ID NO:34 or SEQ ID NO:35.

10. The method of claim 1, wherein the anti-CEACAM1 mAb administered is CM-24.

11. The method of claim 1, comprising administering of at least one additional anti-cancer therapy selected from the group consisting of chemotherapy, radiation, surgery, and immunotherapy.

12. The method of claim 13, wherein the immunotherapy comprises an inhibitor of an immune checkpoint inhibitor.

13. The method of claim 14, wherein the immune checkpoint inhibitor is a PD-1 inhibitor or a PD-L1 inhibitor.

14. The method of claim 1, wherein the biological sample is a biopsy.

15. The method of claim 1, wherein high expression level of CEACAM1 on TILs is predictive of prolonged survival in a subject administered with anti-CEACAM1 mAb.

16. A diagnostic composition comprising an anti-CEACAM1 mAb or an active fragment thereof for use in optimizing treatment in a cancer subject in need of such treatment, the use comprises:
(i) utilizing a diagnostic composition comprising a mAb specific to human CEACAM1 to a biological sample obtained from a subject;
(ii) determining the level of CEACAM1 expression on tumor infiltrating immune cells (TILs) in said biological sample;
(iii) comparing the level of CEACAM1 expression on TILs to a reference value or to a control sample value; and
(iv) optimizing treatment to said subject based on the CEACAM1 expression level on TILs in said sample.

17. A kit for predicting the response of a subject to an anti-CEACAM1 mAb treatment, the kit comprises the diagnostic composition of claim 16; and instruction material directing the correlation between the ratio of the CEACAM1 expression on TILs from the biological sample to the reference or control levels.

18. A method of predicting responsiveness to or selecting a cancer patient amenable to a treatment with an anti-CEACAM1 mAb or active fragment thereof, comprising the steps of: (i) providing a biological sample from the subject; (ii) determining the level CEACAM1 tumor infiltrating immune cells (TILs) in the biological sample of step (i), and (iii) comparing the level of CEACAM1-expressing TILs to a reference value or to a control sample value, wherein an increase in the level of CEACAM1-expressing TILs in said biological sample relative to the reference value or to the control sample value indicates that the subject is likely to respond therapeutically to the anti-CEACAM1 mAb or active fragment thereof.

19. The method of claim 18, wherein the anti-CEACAM1 mAb for treatment is CM-24.

20. The method of claim 18, wherein the level of CEACAM1 expression on the TILs is determined using an anti-CEACAM1 mAb or an active fragment thereof, comprising:
(i) a heavy-chain variable region amino-acid sequence comprising the three CDR sequences set forth in SEQ ID NO:1, SEQ ID NO:2 and SEQ ID NO:3; and
(ii) a light-chain variable region amino-acid sequence comprising the three CDR sequences set forth in SEQ ID NO:4, SEQ ID NO:5 and SEQ ID NO:6.


SEQUENCE LISTING (59 sequences)

SEQ ID NO: 1 - protein, 10 aa, synthetic construct
  REGION 1..10: note = synthetic peptide
GYAFTNNLIE

SEQ ID NO: 2 - protein, 17 aa, synthetic construct
  REGION 1..17: note = synthetic peptide
VINPGSGDTNYNEKFKG

SEQ ID NO: 3 - protein, 11 aa, synthetic construct
  REGION 1..11: note = synthetic sequence
GDYYGGFAVDY

SEQ ID NO: 4 - protein, 11 aa, synthetic construct
  REGION 1..11: note = synthetic peptide
RTSQDIGNYLN

SEQ ID NO: 5 - protein, 7 aa, synthetic construct
  REGION 1..7: note = synthetic peptide
YTSRLHS

SEQ ID NO: 6 - protein, 9 aa, synthetic construct
  REGION 1..9: note = synthetic peptide
QQGKSLPRT

SEQ ID NO: 7 - protein, 25 aa, synthetic construct [sequence omitted]

SEQ ID NO: 8 - protein, 14 aa, synthetic construct
  REGION 1..14: note = synthetic peptide
WVRQAPGQGLEWMG

SEQ ID NO: 9 - protein, 32 aa, synthetic construct
  REGION 1..32: note = synthetic peptide
RVTMTRDTSISTAYMELSRLRSDDTAVYYCAR

SEQ ID NO: 10 - protein, 11 aa, synthetic construct
  REGION 1..11: note = synthetic peptide
WGQGTTVTVSS

SEQ ID NO: 11 - protein, 23 aa, synthetic construct
  REGION 1..23: note = synthetic peptide
DIQMTQSPSSLSASVGDRVTITC

SEQ ID NO: 12 - protein, 15 aa, synthetic construct
  REGION 1..15: note = synthetic peptide
WYQQKPGKAPKLLIY

SEQ ID NO: 13 - protein, 32 aa, synthetic construct
  REGION 1..32: note = synthetic peptide
GVPSRFSGSGSGTDFTFTISSLQPEDIATYYC

SEQ ID NO: 14 - protein, 10 aa, synthetic construct
  REGION 1..10: note = synthetic peptide
FGGGTKVEIK

SEQ ID NO: 15 - protein, 25 aa, synthetic construct
  REGION 1..25: note = synthetic peptide
QVQLVQSGAELKKPGASVKVSCKAS

SEQ ID NO: 16 - protein, 14 aa, synthetic construct
  REGION 1..14: note = synthetic peptide
WVKQAPGQGLEWIG

SEQ ID NO: 17 - protein, 14 aa, synthetic construct
  REGION 1..14: note = synthetic peptide
WVRQAPGQGLEWIG

SEQ ID NO: 18 - protein, 32 aa, synthetic construct
  REGION 1..32: note = synthetic peptide
RATLTADKSINTAYMELSSLTSDDSAVYFCAR

SEQ ID NO: 19 - protein, 32 aa, synthetic construct
  REGION 1..32: note = synthetic peptide
RATLTADKSINTAYMELSRLRSDDTAVYFCAR

SEQ ID NO: 20 - protein, 32 aa, synthetic construct
  REGION 1..32: note = synthetic peptide
RATLTADKSINTAYMELSRLRSDDTAVYYCAR

SEQ ID NO: 21 - protein, 32 aa, synthetic construct
  REGION 1..32: note = synthetic peptide
RATLTADKSISTAYMELSRLRSDDTAVYYCAR

SEQ ID NO: 22 - protein, 32 aa, synthetic construct
  REGION 1..32: note = synthetic peptide
RVTMTADKSISTAYMELSRLRSDDTAVYYCAR

SEQ ID NO: 23 - protein, 11 aa, synthetic construct
  REGION 1..11: note = synthetic peptide
WGQGTSVTVSS

SEQ ID NO: 24 - protein, 15 aa, synthetic construct
  REGION 1..15: note = synthetic peptide
WYQQKPGKAVKLLIY

SEQ ID NO: 25 - protein, 32 aa, synthetic construct
  REGION 1..32: note = synthetic peptide
GVPSRFSGSGSGTDYTLTISSLQQEDIATYFC

SEQ ID NO: 26 - protein, 32 aa, synthetic construct
  REGION 1..32: note = sythetic peptide
GVPSRFSGSGSGTDYTLTISSLQPEDIATYFC

SEQ ID NO: 27 - protein, 32 aa, synthetic construct
  REGION 1..32: note = synthetic peptide
GVPSRFSGSGSGTDYTFTISSLQPEDIATYFC

SEQ ID NO: 28 - protein, 120 aa, synthetic construct
  REGION 1..120: note = synthetic peptide
QVQLVQSGAELKKPGASVKVSCKASGYAFTNNLIEWVKQAPGQGLEWIGVINPGSGDTNYNEKFKGRATLTADKSINTAYMELSSLTSDDSAVYFCARGDYYGGFAVDYWGQGTSVTVSS

SEQ ID NO: 29 - protein, 120 aa, synthetic construct
  REGION 1..120: note = synthetic peptide
QVQLVQSGAEVKKPGASVKVSCKASGYAFTNNLIEWVKQAPGQGLEWIGVINPGSGDTNYNEKFKGRATLTADKSINTAYMELSRLRSDDTAVYFCARGDYYGGFAVDYWGQGTTVTVSS

SEQ ID NO: 30 - protein, 120 aa, synthetic construct
  REGION 1..120: note = synthetic peptide
QVQLVQSGAEVKKPGASVKVSCKASGYAFTNNLIEWVRQAPGQGLEWIGVINPGSGDTNYNEKFKGRATLTADKSINTAYMELSRLRSDDTAVYYCARGDYYGGFAVDYWGQGTTVTVSS

SEQ ID NO: 31 - protein, 120 aa, synthetic construct
  REGION 1..120: note = synthetic peptide
QVQLVQSGAEVKKPGASVKVSCKASGYAFTNNLIEWVRQAPGQGLEWIGVINPGSGDTNYNEKFKGRATLTADKSISTAYMELSRLRSDDTAVYYCARGDYYGGFAVDYWGQGTTVTVSS

SEQ ID NO: 32 - protein, 120 aa, synthetic construct
  REGION 1..120: note = synthetic peptide
QVQLVQSGAEVKKPGASVKVSCKASGYAFTNNLIEWVRQAPGQGLEWIGVINPGSGDTNYNEKFKGRVTMTADKSISTAYMELSRLRSDDTAVYYCARGDYYGGFAVDYWGQGTTVTVSS

SEQ ID NO: 33 - protein, 107 aa, synthetic construct
  REGION 1..107: note = synthetic peptide
DIQMTQSPSSLSASVGDRVTITCRTSQDIGNYLNWYQQKPGKAVKLLIYYTSRLHSGVPSRFSGSGSGTDYTLTISSLQQEDIATYFCQQGKSLPRTFGGGTKVEIK

SEQ ID NO: 34 - protein, 107 aa, synthetic construct
  REGION 1..107: note = synthetic peptide
DIQMTQSPSSLSASVGDRVTITCRTSQDIGNYLNWYQQKPGKAVKLLIYYTSRLHSGVPSRFSGSGSGTDYTLTISSLQPEDIATYFCQQGKSLPRTFGGGTKVEIK

SEQ ID NO: 35 - protein, 107 aa, synthetic construct
  REGION 1..107: note = synthetic peptide
DIQMTQSPSSLSASVGDRVTITCRTSQDIGNYLNWYQQKPGKAVKLLIYYTSRLHSGVPSRFSGSGSGTDYTFTISSLQPEDIATYFCQQGKSLPRTFGGGTKVEIK

SEQ ID NO: 36 - protein, 25 aa, synthetic construct
  REGION 1..25: note = synthetic peptide
QVQLQQSGAELVRPGTSVKVSCKAS

SEQ ID NO: 37 - protein, 14 aa, synthetic construct
  REGION 1..14: note = synthetic peptide
WVKQRPGQGLEWIG

SEQ ID NO: 38 - protein, 32 aa, synthetic construct
  REGION 1..32: note = stnthetic peptide
KATLTADKSSNTAYMQLSSLTSDDSAVYFCAR

SEQ ID NO: 39 - protein, 11 aa, synthetic construct
  REGION 1..11: note = synthetic peptide
WGQGTSVTVSS

SEQ ID NO: 40 - protein, 23 aa, synthetic construct
  REGION 1..23: note = synthetic peptide
DIQMTQTTSSLSASLGDRVTISC

SEQ ID NO: 41 - protein, 15 aa, synthetic construct
  REGION 1..15: note = synthetic peptide
WYQQKPDGTVKLLIY

SEQ ID NO: 42 - protein, 32 aa, synthetic construct
  REGION 1..32: note = synthetic peptide
GVPSRFSGSGSGTDYSLTISNLEQEDIATYFC

SEQ ID NO: 43 - protein, 10 aa, synthetic construct
  REGION 1..10: note = synthetic peptide
FGGGTKLEIK

SEQ ID NO: 44 - DNA, 360 bp, synthetic construct
  misc_feature 1..360: note = Synthetic polynucleotide sequence
  source 1..360: mol_type = other DNA
caggtgcagctggtgcagtctggcgccgagctgaagaaacctggcgcctctgtgaaggtgtcctgcaaggcctccggctacgccttcaccaacaacctgatcgagtgggtcaaacaggccccaggccagggcctggaatggatcggcgtgatcaaccccggctccggcgacaccaactacaacgagaagttcaagggccgggccaccctgaccgccgacaagtccatcaacaccgcctacatggagctgtcctccctgacctccgacgactccgccgtgtacttctgcgccagaggcgactactacggcggcttcgccgtggattactggggccagggcacctccgtgaccgtgtcctca

SEQ ID NO: 45 - DNA, 360 bp, synthetic construct
  misc_feature 1..360: note = synthetic polynucleotide
  source 1..360: mol_type = other DNA
caggtgcagctggtgcagtctggcgccgaggtgaagaaacctggcgcctctgtgaaggtgtcctgcaaggcctccggctacgccttcaccaacaacctgatcgagtgggtcaaacaggccccaggccagggcctggaatggatcggcgtgatcaaccccggctccggcgacaccaactacaacgagaagttcaagggccgggccaccctgaccgccgacaagtccatcaacaccgcctacatggagctgtccagactgaggtccgacgacaccgccgtgtacttctgcgccagaggcgactactacggcggcttcgccgtggattactggggccagggcaccaccgtgaccgtgtcctca

SEQ ID NO: 46 - DNA, 360 bp, synthetic construct
  misc_feature 1..360: note = synthetic polynucleotide
  source 1..360: mol_type = other DNA
caggtgcagctggtgcagtctggcgccgaggtgaagaaacctggcgcctctgtgaaggtgtcctgcaaggcctccggctacgccttcaccaacaacctgatcgagtgggtcagacaggccccaggccagggcctggaatggatcggcgtgatcaaccccggctccggcgacaccaactacaacgagaagttcaagggccgggccaccctgaccgccgacaagtccatcaacaccgcctacatggagctgtccagactgaggtccgacgacaccgccgtgtactactgcgccagaggcgactactacggcggcttcgccgtggattactggggccagggcaccaccgtgaccgtgtcctca

SEQ ID NO: 47 - DNA, 360 bp, synthetic construct
  misc_feature 1..360: note = synthetic polynucleotide
  source 1..360: mol_type = other DNA
caggtgcagctggtgcagtctggcgccgaggtgaagaaacctggcgcctctgtgaaggtgtcctgcaaggcctccggctacgccttcaccaacaacctgatcgagtgggtcagacaggccccaggccagggcctggaatggatcggcgtgatcaaccccggctccggcgacaccaactacaacgagaagttcaagggccgggccaccctgaccgccgacaagtccatcagcaccgcctacatggagctgtccagactgaggtccgacgacaccgccgtgtactactgcgccagaggcgactactacggcggcttcgccgtggattactggggccagggcaccaccgtgaccgtgtcctca

SEQ ID NO: 48 - DNA, 360 bp, synthetic construct
  misc_feature 1..360: note = synthetic polynucleotide
  source 1..360: mol_type = other DNA
caggtgcagctggtgcagtctggcgccgaggtgaagaaacctggcgcctctgtgaaggtgtcctgcaaggcctccggctacgccttcaccaacaacctgatcgagtgggtcagacaggccccaggccagggcctggaatggatcggcgtgatcaaccccggctccggcgacaccaactacaacgagaagttcaagggccgggtgaccatgaccgccgacaagtccatcagcaccgcctacatggagctgtccagactgaggtccgacgacaccgccgtgtactactgcgccagaggcgactactacggcggcttcgccgtggattactggggccagggcaccaccgtgaccgtgtcctca

SEQ ID NO: 49 - DNA, 321 bp, synthetic construct
  misc_feature 1..321: note = synthetic polynucleotide
  source 1..321: mol_type = other DNA
gacatccagatgacccagagcccttccagcctgtccgcctccgtgggcgacagagtgaccatcacctgccggacctcccaggacatcggcaactacctgaactggtatcagcagaaacccggcaaggccgtgaagctgctgatctactacacctcccggctgcactccggcgtgccctccagattctccggctctggctccggcaccgactacaccctgaccatcagcagcctgcagcaggaagatatcgctacctacttctgtcagcagggcaagtctctgcctcggacctttggcggaggcaccaaggtggaaatcaaa

SEQ ID NO: 50 - DNA, 321 bp, synthetic construct
  misc_feature 1..321: note = synthetic polynucleotide
  source 1..321: mol_type = other DNA
gacatccagatgacccagagcccttccagcctgtccgcctccgtgggcgacagagtgaccatcacctgccggacctcccaggacatcggcaactacctgaactggtatcagcagaaacccggcaaggccgtgaagctgctgatctactacacctcccggctgcactccggcgtgccctccagattctccggctctggctccggcaccgactacaccctgaccatcagcagcctgcagcctgaagatatcgctacctacttctgtcagcagggcaagtctctgcctcggacctttggcggaggcaccaaggtggaaatcaaa

SEQ ID NO: 51 - DNA, 321 bp, synthetic construct
  misc_feature 1..321: note = synthetic polynucleotide
  source 1..321: mol_type = other DNA
gacatccagatgacccagagcccttccagcctgtccgcctccgtgggcgacagagtgaccatcacctgccggacctcccaggacatcggcaactacctgaactggtatcagcagaaacccggcaaggccgtgaagctgctgatctactacacctcccggctgcactccggcgtgccctccagattctccggctctggctccggcaccgactacaccttcaccatcagcagcctgcagcctgaagatatcgctacctacttctgtcagcagggcaagtctctgcctcggacctttggcggaggcaccaaggtggaaatcaaa

SEQ ID NO: 52 - protein, 214 aa, synthetic construct
  REGION 1..214: note = synthetic peptide
DIQMTQSPSSLSASVGDRVTITCRTSQDIGNYLNWYQQKPGKAVKLLIYYTSRLHSGVPSRFSGSGSGTDYTLTISSLQPEDIATYFCQQGKSLPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 447 aa, synthetic construct
  REGION 1..447: note = synthetic peptide
QVQLVQSGAEVKKPGASVKVSCKASGYAFTNNLIEWVRQAPGQGLEWIGVINPGSGDTNYNEKFKGRVTMTADKSISTAYMELSRLRSDDTAVYYCARGDYYGGFAVDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 54 - DNA, 1353 bp, synthetic construct
  misc_feature 1..1353: note = synthetic polynucleotide
  source 1..1353: mol_type = other DNA
caggtgcagctggtgcagtctggcgccgaggtgaagaaacctggcgcctctgtgaaggtgtcctgcaaggcctccggctacgccttcaccaacaacctgatcgagtgggtcagacaggccccaggccagggcctggaatggatcggcgtgatcaaccccggctccggcgacaccaactacaacgagaagttcaagggccgggtgaccatgaccgccgacaagtccatcagcaccgcctacatggagctgtccagactgaggtccgacgacaccgccgtgtactactgcgccagaggcgactactacggcggcttcgccgtggattactggggccagggcaccaccgtgaccgtgtcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 55 - DNA, 1344 bp, synthetic construct
  misc_feature 1..1344: note = synthetic polynucleotide
  source 1..1344: mol_type = other DNA
caggtgcagctggtgcagtctggcgccgaggtgaagaaacctggcgcctctgtgaaggtgtcctgcaaggcctccggctacgccttcaccaacaacctgatcgagtgggtcagacaggccccaggccagggcctggaatggatcggcgtgatcaaccccggctccggcgacaccaactacaacgagaagttcaagggccgggtgaccatgaccgccgacaagtccatcagcaccgcctacatggagctgtccagactgaggtccgacgacaccgccgtgtactactgcgccagaggcgactactacggcggcttcgccgtggattactggggccagggcaccaccgtgaccgtgtcctcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaatgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatga

SEQ ID NO: 56 - DNA, 645 bp, synthetic construct
  misc_feature 1..645: note = synthetic polynucleotide
  source 1..645: mol_type = other DNA
gacatccagatgacccagagcccttccagcctgtccgcctccgtgggcgacagagtgaccatcacctgccggacctcccaggacatcggcaactacctgaactggtatcagcagaaacccggcaaggccgtgaagctgctgatctactacacctcccggctgcactccggcgtgccctccagattctccggctctggctccggcaccgactacaccctgaccatcagcagcctgcagcctgaagatatcgctacctacttctgtcagcagggcaagtctctgcctcggacctttggcggaggcaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 57 - protein, 120 aa, synthetic construct
  REGION 1..120: note = synthetic peptide
QVQLVQSGAEVKKPGASVKVSCKASGYAFTNNLIEWVRQAPGQGLEWMGVINPGSGDTNYNEKFKGRVTMTRDTSISTAYMELSRLRSDDTAVYYCARGDYYGGFAVDYWGQGTTVTVSS

SEQ ID NO: 58 - protein, 107 aa, synthetic construct
  REGION 1..107: note = synthetic peptide
DIQMTQSPSSLSASVGDRVTITCRTSQDIGNYLNWYQQKPGKAPKLLIYYTSRLHSGVPSRFSGSGSGTDFTFTISSLQPEDIATYYCQQGKSLPRTFGGGTKVEIK

SEQ ID NO: 59 - protein, 450 aa, synthetic construct
  REGION 1..450: note = synthetic peptide
QVQLVQSGAEVKKPGASVKVSCKASGYAFTNNLIEWVRQAPGQGLEWIGVINPGSGDTNYNEKFKGRVTMTADKSISTAYMELSRLRSDDTAVYYCARGDYYGGFAVDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK